SEQUENCE LISTING (16 sequences)

SEQ ID NO: 1 - DNA, 2211 bp, Artificial Sequence
  synthetic construct
atggctgccgatggttatcttccagattggctcgaggacaacctctctgagggcattcgcgagtggtgggacttgaaacctggagccccgaaacccaaagccaacgagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaacctcttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggttgcctccctccgtccccggcggacgtgttcatgattcctcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaatacaaggacttgctgtttagccgggggtctccagctggcatgtctgttcagcccaaaaactggctacctggaccctgttaccggcagcagcgcgtttctaaaacaaaaacagacaacaacaacagcaactttacgtggactggtgcttcaaaatataaccttaatgggcgtgaatctataatcaaccctggcactgctatggcctcacacaaagacgacaaagacaagttctttcccatgagcggtgtcatgatttttggaaaggagagcgcgggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcatcattcatcacccagtattccacaggacaagtgagcgtggagattgaatgggagctgcagaaagaaaacagcaaacgctggaatcccgaagtgcagtatacatctatctatgcaaaatctgccaacgttgatttcactgtggacaacaatggactttatactgagcctcgccccattggcacccgttacctcacccgtcccctgtaa

SEQ ID NO: 2 - DNA, 2211 bp, Artificial Sequence
  synthetic construct
atggctgccgatggttatcttccagattggctcgaggacaacctctctgagggcattcgcgagtggtgggacttgaaacctggagccccgaaacgcaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaacctcttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggatgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctaccaaacgctcaacaatggcagccaggcagtgggacggtcatccttttactgcctggaatatttcccatcgcagatgctgagaacgggcaataactttaccatcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccgtgttaccggcagcagcgcgtttctaaaacataaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggcgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 3 - DNA, 5906 bp, Artificial Sequence
  synthetic construct
atgcccaacaacctgacggactgcgaggacggcgatgggggagccaacccgggtgatggcaaccccaaggaaagcagtcccttcatcaacagcaccgacacagagaagggaaaggagtatgatggcaagaacatggccttgtttgaggaggagatggacaccagccctatggtgtcctccttgctcagtggcctggccaactacaccaacctgccccagggaagtagggagcatgaagaggcagaaaacaatgagggtggaaaaaagaagccggtgcaggccccacgcatgggcaccttcatgggcgtgtacctgccgtgcctgcagaacatctttggcgtcatcctcttcctgcggctcacctgggtggtgggcattgcaggcatcatggagtccttctgcatggtgttcatctgctgctcctgtacgatgctcacggccatctccatgagtgcaattgcaacgaatggtgttgtgcctgctggtggctcctactacatgatttccaggtctctgggcccagagtttgggggtgccgtgggcctctgcttctacctgggcactacctttgcaggagccatgtacatcctgggcaccatcgaaatcctgctggcttacctcttcccagccatggccatcttcaaggcagaagatgccagtggggaggcagcagccatgctgaacaacatgcgtgtttacggcacctgtgtgctcacctgcatggccactgtggtgtttgtgggtgtcaagtatgtcaacaagtttgcccttgtcttcctgggttgtgtcatcctctccatcctggccatctatgctggggtcatcaagtctgccttcgacccacccaacttcccgatctgcctcctgggtaaccgcacgctgtctcgccatggctttgatgtctgtgccaagctggcttgggaaggaaatgagacggtgaccacacggctatggggccttttctgctcctctcgcttcctcaacgccacctgtgatgaatacttcacccgaaacaatgtcacagagatccagggcatccctggtgctgccagtggcctcatcaaagagaacctctggagctcctacctgaccaagggcgtgattgtggagaggagtgggatgacctcggtgggcctggccgatggcactcctatcgacatggaccacccttatgtcttcagtgatatgacctcctacttcaccctgctggttggcatctacttcccctcagtcacagggatcatggctggttctaaccgctctggggacctgagggatgcccagaagtcaatccccactggcaccatcctggccatcgccaccacctctgctgtctacatcagctccgttgttctgtttggggcctgcattgagggggtcgtcctgcgggacaagtttggcgaagctgtgaatggcaacctcgtggtgggcactctggcctggccatctccatgggtaattgtcatcggatccttcttctccacctgtgggctgggctgcagagcctcacgggggccccacgcctgctgcaggccatctcgagggatggcattgtgcccttcctgcaggtctttggccatggcaaggccaatggagagccgacctgggccctgctcctgactgcctgcatctgcgagattggcatcctcattgcatccctcgacgaggtggcccccatcctctctatgttcttcctgatgtgctacatgtttgtgaatctggcctgtgcagtgcagacgctgctgaggacacccaactggaggccacgctttcgatattaccactggaccctctccttcctgggcatgagcctctgcctggccctcatgttcatctgctcctggtattatgcactggtagccatgctcattgctggactcatctacaagtacattgagtaccgtggggcagagaaggagtggggcgatgggatacgaggtctgtctctcagtgcggctcgctatgccctcttacgcctggaggaagggcccccacacaccaagaactggaggccacagctgctggtgctggtgcgtgtggaccaagaccagaatgtggtgcacccccagctgctctcactgacctcccagctgaaggcagggaagggcctgaccatcgtgggctctgtccttgagggcacctttctggaaaatcatccacaggcccagcgggcagaagagtctatcaggcgcctgatggaggcagagaaggtgaagggcttctgccaggtggtgatctcctccaacttgcgtgatggcgtgtcccatctgatccagtccgggggcctcggggggctgcagcacaacactgtgcttgttggctggccccgcaactggcgccagaaggaagatcatcagacgtggaggaacttcattgagctggtccgggaaaccacagctggccacttagccctgctggtcaccaagaacgtttccatgtttcctgggaaccctgagcgcttctctgagggcagcatcgacgtttggtggattgtgcacgatggaggcatgctcatgctgctgcccttcctgctgcggcaccacaaggtctggcggaagtgcaagatgcgtatcttcactgtggcccagatggatgacaatagcatccagatgaagaaggatctgaccacatttctgtatcatttacgcatcactgcggaggtcgaggtggtggagatgcatgagagcgacatctcagcttacacctatgagaagacgttggtgatggagcagcgttcccagatcctcaaacagatgcatttaaccaagaatgagcgggagcgggagatccagagtatcacagatgagtcacgaggctcaatccggagaaagaatccagccaacacgcggctccgcctgaacgtcccagaagagacggctggtgacagtgaagagaagccagaggaggaggtgcagctgatccacgatcagagtgctcccagctgccccagcagctccccgtccccaggggaggagcctgagggggaaggggagacagatccggagaaggtgcatctcacctggaccaaggacaagtcggtggcagagaagaataagggccccagtcctgtctcctctgagggcatcaaggacttcttcagcatgaagccggagtgggagaacttgaaccagtccaacgtgcggcgcatgcacacggccgtgcggctgaacgaggtcatcgtgaagaaatcccgggacgccaagcttgttttgctcaacatgcctgggcctccccgcaaccgcaatggtgatgaaaactacatggagtttctcgaggtcctcacagagcacctggaccgggtgatgctggtccgcggtggtggccgagaggtcatcaccatctactcctgagaaccaggtcctgccacccgggcccgagcgcgcccggcccgcggctccggagccctcgccgcgccccccgccgctgtcaccgtttacatacagaccctgtgcccgtgtcctggccccttaccccgctgcctgaagcccggaggccacgcctgttggggctgattcggagagggcgccccgccgcgcagagaccagagctcctcagtgccagtttggcccctgggtcttcgctgccctttttctaagcccggcctcgtctcgccggaggagacgctgcaataaaggttgggagaaggcgcggaaaggagaggagctggggccttggggacccccaggtagtccatgcggcccattcctccccttcccactcccgccgcggtcctcgctctgcgctcctccggcgctgctccctggctcccggcggcccggaggcccgcggggtgggaaggccgcgcttgccgtctccgccgccccttctcgccgagccgtggggcgcgggcggccgagcctatacatagtgtacaggagacatcgcgtgtatttttaacgtccccatatttatgtgactagaagcgcaacagacttctcgccatagtcgagctctcccgctgggggcactgcggggaggcgaggcctcgggaagctgaattttccttgacgtccaagagtttgagagcgaaagtgctttaggcccaggcgggggtcgtggcctcgttccctcgacacctccgtcctgctctcgcctcttcgccctttccgcgcgcccttggcttcccaccctcctctccagtccttttccgagatgaggtgagacaagggtccaacttttcctggattcgcctcccagcggacgtgagcttccactgcggctgcagagacgcgagcaacctcttctcatcggctcttatgcaagttggggccaggataggggaggggtgctcctcaagaggaagaaaccgagaggcccgcgccccaccgaggaagccccgccccggtgccttcgctggggagcaggcgtctctcctcagtcggcttgtcgcctgctccccgtatcccatggctcctcgccaaagactgaaattgtggagctggagggcgccccctccccggagtttcctccctgggacaagtgagggaggagggggccgattctggtttaggggccggacccactgagaggccccagagccgcccgtgatgttcctcccccgtccccatctggcagctcctgtctcgcctgagggacccagccgccttctccgtgctctggggccgggcctcgctgcttagcagcggcctctagctccgtctcccggggacctgggcctgagggagggctggagtcagcacgcgctttgtccttagcgcctgtctgctctcctctaactaggacccagggcctttggcttccccagctcatccttggcccttccgctccaccagcctggtctgaggcgtgctctgtccttagagaaggcgcggtggccgggttcccttcccctagggcacattactaagggggtcaggcactgcatgctcgttccagcaccatctgggactgggtacagtacctccagccccagggccctgacctgcgcacctagcttgacatctcacgcacctcccagagctggcgccactgagtaatccggacctcaccacctcttttcctttgagcccaaggcagagctagagctggagctggcgccacccagacagcgtcaggtgtggctggggtaggtttggaggtctgccagttacgccaagtcccctctgagattcgatcaggggactggatagattctttcaggtactcaatcaggaagctggaggtgttagacaccagccccctgcatccttcagtagacctccctctgaacaccacagccaggtcctgccttctgggggcctgaatattccagagctgatgtgatgggctgtgcagaagggggctgtatcaacatcaattagggaaccaaagttgcactatctgggcccagattgtctggttggcaagagcaaagtttccgttgatgaaacagacatcccacaacaaaaacccaagttttctgtgctacatgtgcaatatttgttatgaatgttatcacaagtcattcatcaagttatctttataatcactgtagttagatgtttcatgtccattcaagtgacttttattctgagtgcaatatttcaatagccttgtagtgataactagtgttgcttttgtttagatgatctatgtgcagggcaatgcaatgaagttgaaaccccttggtaataggagaggttgcaaaccaaatcaagagtatttattactattactgctattattattaggcctgcctttaattttcagtgtaagtgttcagtatgccgcatcctgcctcagtattgatcttgtgttctttgtgccaatatgaaaaggagagggttggttctttcctttattgttgaatgctcccatttaatgctttatggcttttactgtattacttttttagactcccgtctgcacaaaatgcaataaaaataattttattataaaaaaaaaaa

SEQ ID NO: 4 - protein, 1116 aa, Artificial Sequence [sequence omitted]

SEQ ID NO: 5 - DNA, 7985 bp, Artificial Sequence
  synthetic construct
agaaatggaccgagcggacccgccgccgcacgcaccctgctccactccaagctcctaagggctcctggcgcgccgcgtagccttggcgaggtccgcgctggggtgcggagagcgaagggaactggagagccatgtagatccaggctctcgcccgcccgcctccttcgggatcgaatcaagggctcccatagtgttaggagggggcgagagtgctgtttatcgtcatttgcctcggagcttcgagagagggtggtattttgcttttccgccccgcatcctccggaactccctgcaccggagagaggacggcgtctccaggttgctggcaaccggtgagaatgggggtagggaaggaacattttcgccgtagctgctccgtaaagcgattgtccaactgagaggggcgtcggacgagtggaccagggcggcgagtttgcccggcgcgtctcggatgctgctgcggcggccgccgcggctcccgccagggcactgcaaagacgacctgccgcattcccactcgggctctccgctgactcagcaccgcccctgcgccaagccagccggccagcgtgcatcgatcgccctggtgggagcttagaaggcggcaggcgaagaggggtaggaggggggagagccgaggagaagcagagagggtggcaggcgtggggatctgccgagccggcactgcaccgggtcctaggaaggctctcggaggggaggggaggccagggcgacccccgaagcaatggcccagtccgctagaacggcactgcgttaaggcacctgggatcaggaagaaatatctaaacaacaacaacagaaaaccaacaaacccccaaacccaaacccaaccctctgcaaaaagctgcacccggcccgcaggcgagggggattccaaactgagtgaaaggcagggtggaggggaaggcagcgagaggcaaagtcgcagatctcccgacctgctcgtgttgaagcacctccccctgggcgtgagggagacgcgcgctccggtgggggggccgcttgggtcccccccacccctggtccctggctgcttcccaccccgggctctctcctggcctcccacccccgcgcccggcttccaccatgacggtgatgtctggggagaacgtggacgaggcttcggccgccccgggccacccccaggatggcagctacccccggcaggccgaccacgacgaccacgagtgctgcgagcgcgtggtgatcaacatctccgggctgcgcttcgagacgcagctcaagaccctggcgcagttccccaacacgctgctgggcaaccctaagaaacgcatgcgctacttcgaccccctgaggaacgagtacttcttcgaccgcaaccggcccagcttcgacgccatcctctactactaccagtccggcggccgcctgcggaggccggtcaacgtgcccctggacatgttctccgaggagatcaagttttacgagttgggcgaggaggccatggagaagttccgggaggacgagggcttcatcaaggaggaggagcgccctctgcccgagaaggagtaccagcgccaggtgtggctgctcttcgagtaccccgagagctcggggcccgccagggtcatcgccatcgtctccgtcatggtcatcctcatctccatcgtcatcttttgcctggagacgctccccgagctgaaggatgacaaggacttcacgggcaccgtccaccgcatcgacaacaccacggtcatctacaattccaacatcttcacagaccccttcttcatcgtggaaacgctgtgtatcatctggttctccttcgagctggtggtgcgcttcttcgcctgccccagcaagacggacttcttcaaaaacatcatgaacttcatagacattgtggccatcattccttatttcatcacgctgggcaccgagatagctgagcaggaaggaaaccagaagggcgagcaggccacctccctggccatcctcagggtcatccgcttggtaagggtttttagaatcttcaagctctcccgccactctaagggcctccagatcctgggccagaccctcaaagctagtatgagagagctagggctgctcatctttttcctcttcatcggggtcatcctgttttctagtgcagtgtactttgccgaggcggaagaagctgagtcgcacttctccagtatccccgatgctttctggtgggcggtggtgtccatgaccactgtaggatacggtgacatgtaccctgtgacaattggaggcaagatcgtgggctccttgtgtgccatcgctggtgtgctaacaattgccctgcccgtacctgtcattgtgtccaatttcaactatttctaccaccgagaaactgagggggaagagcaggctcagttgctccacgtcagttcccctaacttagcctctgacagtgacctcagtcgccgcagttcctctactatgagcaagtctgagtacatggagatcgaagaggatatgaataatagcatagcccattatagacaggtcaatatcagaactgccaattgcaccactgctaaccaaaactgcgttaataagagcaagctactgaccgatgtttaaaaaacaaaggcaagcaaacaaaaaagccccacttagcagctcaaaagacttaaaaaacaaaacagaaaacctagtgactcatgtcacgctttgtagatactttactaagtagacttggaatgctctatttaactgtcaatgcgttgttgcattgaggattttgggggtggtgaaccagaagctttcaagatccatgacaaaataaactattttccttttattaaaaaatgggaaaagagagagtattttctaaaactggcttaaaaagattcagtccacgaactagtctaggtaaaataataatcatatgcttccccaaactgaaacatttttaatgctttggtttctttaacttttttaaaaactcagaacaagatgatcacttagaaatatgaaattgaaattcgcatgggactccagtaaaacatctttgcaaactgcgtagcacattgaagacagtgcatcagatgtattatatgtaacatgatagaccagccaaaatggacaatgaatagatatttttatttcgatcaactgaactgcatattacaaggtgaaaaaagaaaactccgattacttaagactggttcacaaagcaccttataaattggatactggtcctgatctgtagggatttccccctgggcccattctctttctaatccagattattctctaagaaaaagttaactgaattaaattaattgattcttctgcagtgccgctaaatggtctcaactgcagatgagccaaatacaggtcttttctcaccaggcctgcactccgacccctggctttcagaactggatgtaaaaccttagcctccttattgcaagagagcacaaatgaagttaaatgtaagcatgtttgaatctgatacaatttattttataatcgcatgctgagaagttaacccagacaataggggataagcttaagttgaaatcgattcttctaaaaatagatcctttttcatttgcattcaccaaaagtgcactcctccatttattaactattttattagtaaataaagtactgtatttaagtgcatatgttagtcagatgggaacaataactttttggagctcaaagcatgttctcttattcagcattatggcctatttgactaagatgtaccttgaattaattaatgcatgatttcagtaataaaaattttaaaagtaataaaaattacaagtctgtggggtgaaaggcccaatagaaattatggggggtgggggtgggggcactcagtcaattttcctgcctttgctcagggaaataccaggtttttgtgcaggtataggcggagagaggaccaatatgcccatcccttaaagggaagccatgtgaaaaactcaataagtcatcaaagtacatatagcaacacctaagaacaagtattctttctagctgaagacaaacacaagcaacacaaacaagcaaacaaacaaacaaacaaaaaggtgcaatactgcatgttttttggtgcattcttaggatgtaaatgaaaatgtttctctattatatgcatccgaagcagagctgatttttttttctttgcagtcattctttgaagtctgtagagacttcagccctccccttgaggctccctgaagaaactaaaccaattgatttaatagttgcttagtgcctttatcctgtacccacagtgaactgtagaaagtgcctccttaacacagctgagaagttaggtagcaaaagtggggaagggttggggcacagaccttttgctttttctttttccattctcgctctctcatttcaccactgtgagaagaccacaccaccctaaaccctggagaggagagacccaggagggtgctgtctctctggccatctactagcattggtccctttgacagcctgacgctggatgtgaactgagacccatctttgaactggacatgaactgtgaacttgttttttcctctctccaccaaagccaagataaactttttgggaatttgtttcctatcgagggccactttggacacacaaggcttcctcaggtccagtgtagtgctcctggcacctttccttatttttttctctgtcggtaacagcactttgcaaatctctctgacggtccagtcttttcaggcattgttgtggatgtgggaacactcagttcataataacctttcctaggccttccctcctggtctacccctttcagatatttcctgatgcccctatgatcttcccacctggcagtcacttcacaggttgaacatctaacttctgctgcccccccgttacccagcccagagaatggtggggaccctgttcctggctgaaagagagccacggaacacaggcctctggagctcggcagctgcccaccggtggagaggtactcacagcctttcaaaggaccctgaggtggggaatcttcattctgcacttagcatgtggctgcctgttacctgacattctggcccagcttcttctgaaaatctgtcctgcttctcccacccctttcccccatcctgtcctagaactagcagtgaggcaatcaccctagaaactcgagttacacccattctggctaactcgattaaaagaaagaacatggatatcttattttcctatgtgacttttgtgaatctgtgagatgaacacaacacacatcgtggaagatgaggggccaagaactgcgcagcatccgactacagggcattaaaccctcccatgtgatgtctccttctcgtctgaaccttaactcattctggcgattcctttcctacttgcttaaaaatccccaattaggaaaaaaatgccaacctcctcccaggcttggggttatcttgatctttcatttcaacgttgaggtctagtgcacacagaacttgaaacaccgcactctgtcaacagcaataatccactcagtactgtgggatggatgggttaatggatgagaaaacggcaccaagactgactttgggtatttggtatatttaccgtggttacactctggactggttgattcttctgcatgtgatatagaaaaaagtgctgcatgcggtgaacctgtcagtctgggactgggggtgagttgttgttatgagtttggggtggtgtgaggaaagtgaatgctgcttagctcatctgtagctcattgcaatgaattcagtaagaatggagtacagggattatctgtgtagcataggcatgcaatgtttgaccaagctcttaccctcgcactgtaatgtgttgaaatgtctttgtagacctgaaggtgcacttaacaaaactgcctattaagggatgactattttttggtttacttattcatatttattttagcagcctttttaccttttcttccccttcctaggcatggagctgtaacagctcatgtcctgactatgtgttttctccagaaggagaagacttctgatgtgctgatagctataattcctctcttccagtctattggggccctagttcaatagggtggcaatagaagagttggtcacaccagggctgttagccatcccagaatctctgaagtggttaactcacctgaagtgatctgaatcagagagaccaaagacattcatttcctctgtcctcagatttctagaagacaaattctagccaggaaaactttctgtttttgcatctcccttttcccatttgtgcccaactccccttgcactccctggagacttgagttctgattttcagttatatcagctcattgggagtgtgttcgtgggtgagccttgcagaatcagataaaccaatagcaagtccttctccaggattctggccagtgggcagtcatttccctgaaatgaattgtagggcaggtttagattccactatggtggacgtgaagcacaggtggtgtcacaatcttgatttgcttgagaattaaaaaaatacatgtaaatggtaaatgaggaatacatttttttaaagtagaaatttggtttagcctataaaaggccttcttcacattgtgtatagttacacgtttttcaaattaggtaccatctaaaagtcaataaaaatagtttcctagccctctcatttaatataggagacctcaaaacacatgctttttaacaattttacattttaattcttagtttggtagattggattgaaagaaaggagaaagaacattagcagaaggcactttcccattttcttccaggaatgacttattcctggggtggtgagaattggtgggtggtaaccatccatagtataaaattgttagaaagaatataaactgccaaacaagcatgttatcttcaggcttttccaagcaagaatgaagtcttttgatgtttatgttcattttaagaagacaaacaaactaaaattttaagaccagacacaacccaagttgaattgtgatctcaggaggtgaactctcattcaccatgtggcatattactacctgtatttatatcatggaatttcagggtatatgtgaacatgtctagtatgactcaggtaaacctttaaagaatgtatgttacttaccatttttgtaaagaagcaaacaggagctgaattgttaaccaaaactgttccattaccttgggtcactgtgcaaactaattcagggtacagatataaagtttagaagccttgggattaaatgccagcctctttgctcaggcaaaatgaccctgggctttcttgggaagtccagcatgtatgtaaggggtgaggccctgctgacctcagggcttttcgctttaaggagattaaccctagtcacagtgattttcatttaggagctaactaggagtttactctttatgacgtgaaacttcaaagagtatagaaaacttttgtaccacaatcaaagaagaagaaatggtgtatggaaagaaaacaaaacaaaacaagaaatctcttgtaaaatattccaggtcaaagttgtctcctctccaaaccttgcagaagcacctttcttctcttcagcgcactgttttgggactgtttatgcagcagatgtaagtagacaacatggactccatgtgacatgcctctaatagtaaagataaagtattactgaggttaaaaataaaaattgagtagtattaatttaaagtgcaccatcaggacaacaaaccatttaagctgaaaaaacgctattttatttcttgagtttgccagttgcttccaccttgagttaaggacgtgtctcatcttcacctactgcgcattctcccttctctaactgtgtaatatgtcaggtcaaggacattgaatgttatgaattgagaacctaattgatgcgcatagttttcatctatgcaattttacttgcttctgtcactttacgatctgttcatatttggcatcaattaaagatactttttaaggatcttatcaaggaa

SEQ ID NO: 6 - protein, 495 aa, Artificial Sequence
  synthetic construct
MTVMSGENVDEASAAPGHPQDGSYPRQADHDDHECCERVVINISGLRFETQLKTLAQFPNTLLGNPKKRMRYFDPLRNEYFFDRNRPSFDAILYYYQSGGRLRRPVNVPLDMFSEEIKFYELGEEAMEKFREDEGFIKEEERPLPEKEYQRQVWLLFEYPESSGPARVIAIVSVMVILISIVIFCLETLPELKDDKDFTGTVHRIDNTTVIYNSNIFTDPFFIVETLCIIWFSFELVVRFFACPSKTDFFKNIMNFIDIVAIIPYFITLGTEIAEQEGNQKGEQATSLAILRVIRLVRVFRIFKLSRHSKGLQILGQTLKASMRELGLLIFFLFIGVILFSSAVYFAEAEEAESHFSSIPDAFWWAVVSMTTVGYGDMYPVTIGGKIVGSLCAIAGVLTIALPVPVIVSNFNYFYHRETEGEEQAQLLHVSSPNLASDSDLSRRSSSTMSKSEYMEIEEDMNNSIAHYRQVNIRTANCTTANQNCVNKSKLLTDV

SEQ ID NO: 7 - DNA, 1496 bp, Artificial Sequence
  synthetic construct
gaattcgcaattaaggagataataggtatgccgatcaccatcaacaacttccgttactccgacccggttaacaacgacaccatcatcatgatggaaccgccgtactgcaaaggtctagacatctactacaaagccttcaaaatcaccgaccgtatctggatcgttccggaacgttacgaatttggtaccaaaccggaagacttcaacccgccgtcctccctgatcgaaggtgcttccgaatactacgacccgaactacctgcgtaccgactccgacaaagaccgtttcctgcagaccatggttaaactgttcaaccgtatcaaaaacaacgttgctggtgaagctctgctggacaaaatcatcaacgctatcccgtacctgggtaactcctactccctgctggacaaattcgacaccaactccaactccgtttccttcaacctgctggaacaggatccgtccggtgctaccaccaaatccgctatgctgaccaacctgatcatcttcggtccgggtccggttctgaacaaaaacgaagttcgtggtatcgttctgcgtgttgacaacaaaaactacttcccgtgccgtgacggtttcggttccatcatgcagatggctttctgcccggaatacgtaccgaccttcgacaacgttatcgaaaacatcacctccctgaccatcggtaaatccaaatacttccaggacccggctctgctgctgatgcacgaactgatccacgttctgcacggtctgtacggcatgcaggtttcctcccacgaaatcatcccgtccaaacaggaaatctacatgcagcacacctacccgatctccgctgaagagctcttcaccttcggtggtcaggacgctaacctgatctccatcgatatcaaaaacgacctgtacgaaaaaaccctgaacgactacaaagctatcgctaacaaactgtcccaggttacctcctgcaacgacccgaacatcgacatcgactcctacaaacagatctatcagcagaagtatcagtttgataaggattccaacggccagtatatagtgaacgaggataagtttcagatactgtataactccataatgtatggctttactgagatagagctgggcaagaagtttaacataaagactcgcttaagctatttttccatgaaccacgatcctgtgaagatacctaacctgctggatgatactatatataacgatactgagggctttaacatagagtccaaggatctgaagtccgagtataagggccagaacatgagggtgaacactaacgcctttaggaacgtcgacggttccggtctggtttccaaactgatcggtctgtgcaaaaaaatcatcccgccgaccaacatccgtgaaaacctgtacaaccgtaccgcttaatttaaagcagctgcgatcgtaactaagtaataaacggtcgtaacacaacaattgcattccggggttttatgtttcaggttgggggtggtaagctt

SEQ ID NO: 8 - protein, 457 aa, Artificial Sequence
  synthetic construct
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTA

SEQ ID NO: 9 - DNA, 1452 bp, Artificial Sequence
  synthetic construct
atggccaacttcacacctgtcaatggcagctcgggcaatcagtccgtgcgcctggtcacgtcatcatcccacaatcgctatgagacggtggaaatggtcttcattgccacagtgacaggctccctgagcctggtgactgtcgtgggcaacatcctggtgatgctgtccatcaaggtcaacaggcagctgcagacagtcaacaactacttcctcttcagcctggcgtgtgctgatctcatcataggcgccttctccatgaacctctacaccgtgtacatcatcaagggctactggcccctgggcgccgtggtctgcgacctgtggctggccctggactgcgtggtgagcaacgcctccgtcatgaaccttctcatcatcagctttgaccgctacttctgcgtcaccaagcctctcacctaccctgcccggcgcaccaccaagatggcaggcctcatgattgctgctgcctgggtactgtccttcgtgctctgggcgcctgccatcttgttctggcagtttgtggtgggtaagcggacggtgcccgacaaccagtgcttcatccagttcctgtccaacccagcagtgacctttggcacagccattgctggcttctacctgcctgtggtcatcatgacggtgctgtacatccacatctccctggccagtcgcagccgagtccacaagcaccggcccgagggcccgaaggagaagaaagccaagacgctggccttcctcaagagcccactaatgaagcagagcgtcaagaagcccccgcccggggaggccgcccgggaggagctgcgcaatggcaagctggaggaggcccccccgccagcgctgccaccgccaccgcgccccgtggctgataaggacacttccaatgagtccagctcaggcagtgccacccagaacaccaaggaacgcccagccacagagctgtccaccacagaggccaccacgcccgccatgcccgcccctcccctgcagccgcgggccctcaacccagcctccagatggtccaagatccagattgtgacgaagcagacaggcaatgagtgtgtgacagccattgagattgtgcctgccacgccggctggcatgcgccctgcggccaacgtggcccgcaagttcgccagcatcgctcgcaaccaggtgcgcaagaagcggcagatggcggcccgggagcgcaaagtgacacgaacgatctttgccattctgctggccttcatcctcacctggacgccctacaacgtcatggtcctggtgaacaccttctgccagagctgcatccctgacacggtgtggtccattggctactggctctgctacgtcaacagcaccatcaaccctgcctgctatgctctgtgcaacgccacctttaaaaagaccttccggcacctgctgctgtgccagtatcggaacatcggcactgccaggcgggatccaccggtc

SEQ ID NO: 10 - protein, 484 aa, Artificial Sequence
  synthetic construct
MANFTPVNGSSGNQSVRLVTSSSHNRYETVEMVFIATVTGSLSLVTVVGNILVMLSIKVNRQLQTVNNYFLFSLACADLIIGAFSMNLYTVYIIKGYWPLGAVVCDLWLALDCVVSNASVMNLLIISFDRYFCVTKPLTYPARRTTKMAGLMIAAAWVLSFVLWAPAILFWQFVVGKRTVPDNQCFIQFLSNPAVTFGTAIAGFYLPVVIMTVLYIHISLASRSRVHKHRPEGPKEKKAKTLAFLKSPLMKQSVKKPPPGEAAREELRNGKLEEAPPPALPPPPRPVADKDTSNESSSGSATQNTKERPATELSTTEATTPAMPAPPLQPRALNPASRWSKIQIVTKQTGNECVTAIEIVPATPAGMRPAANVARKFASIARNQVRKKRQMAARERKVTRTIFAILLAFILTWTPYNVMVLVNTFCQSCIPDTVWSIGYWLCYVNSTINPACYALCNATFKKTFRHLLLCQYRNIGTARRDPPV

SEQ ID NO: 11 - DNA, 2211 bp, Artificial Sequence
  synthetic construct
atggctgccgatggttatcttccagattggctcgaggacaacctctctgagggcattcgcgactggtgggacttgaaacctggagccccgaaacccaaagccaaccagcaaaagcaggacgacggccggggtctggtgcttcctggctacaagtacctcggaccctttaacggactcgacaagggggagcccgtcaacgcggcggatgcagcggccctcgagcacgacaaggcctacgaccagcagctcaaagcgggtgacaatccgtacctgcggtataaccacgccgacgccgagtgtcaggagcgtctgcaagaagatacgtcttttgggggcaacctcgggcgagcagtcttccaggccaagaagagggtactcgaaccttttggtctggctgaggaaggtgctaagacggctcctggaaagaaacgtccggtagagcagtcgccacaagagccagactcctcctcgggcattggcaagacaggccagcagcccgctaaaaagagactcaattttggtcagaccggcgactcagagtcagtccccgacccacaacctctcggagaacctccagcaacccccgctgctgtgggacctactacaatggcttcaggcggtggcgcaccaatggcagacaataatgaaggcgccgacggagtgggtaatgcctcaggaaattggcattgcgattccacatggctgggcgacagagtcatcaccaccagcacccgaacatgggccttgcccacctataacaaccacctctacaaggaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggacacggaataccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgatgagaacgggcaataactttaccttcagctacacattcgaggacgcgcctttccacagcagctacgcgcacagccagagcctggaccggctgatgaatcctctcatcgaccagtacttgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacttgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 12 - protein, 736 aa, Artificial Sequence
  synthetic construct
MAADGYLPDWLEDNLSEGIRDWWDLKPGAPKPKANQQKQDDGRGLVLPGYKYLGPFNGLDKGEPVNAADAAALEHDKAYDQQLKAGDNPYLRYNHADAECQERLQEDTSFGGNLGRAVFQAKKRVLEPFGLAEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKEISSASTGASNDNHYFGYSTPWGYFDFNRFHCHFSPRDWQRLINNNWGFRPKRLNFKLFNIQVKEVTTNDGVTTIANNLTSTVQVFSDTEYQLPYVLGSAHQGCLPPFPADVFMIPQYGYLTLNNGSQAVGRSSFYCLEYFPSQMMRTGNNFTFSYTFEDAPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 13 - DNA, 2211 bp, Artificial Sequence
  synthetic construct
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 14 - protein, 736 aa, Artificial Sequence
  synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 15 - protein, 736 aa, Artificial Sequence
  synthetic construct
MAADGYLPDWLEDNLSEGIREWWDLKPGAPKPKANEQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPSPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQYKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSIYAKSANVDFTVDNNGLYTEPRPIGTRYLTRPL

SEQ ID NO: 16 - protein, 735 aa, Artificial Sequence
  synthetic construct
MAADGYLPDWLEDNLSEGIREWWDLKPGAPKRKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWMGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYQTLNNGSQAVGRSSFYCLEYFPSQMLRTGNNFTISYTFEDVPFHSSYAHSQSLDRLMNPLIDQYLYYLNRTQNQSGSAQNKDLLFSRGSPAGMSVQPKNWLPGPCYRQQRVSKTTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMAWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

(19) United States
(12) Patent Application Publication
(10) Pub. No.: US 2023/0332178 A1
(43) Pub. Date: Oct. 19, 2023

(54) GENE THERAPY FOR NEUROMUSCULAR AND NEUROMOTOR DISORDERS

(71) Applicant: UCL BUSINESS LTD., London (GB)

(72) Inventors: Robert Morris Brownstone, London (GB); Andrew John Murray, London (GB)

(21) Appl. No.: 18/016,265

(22) PCT Filed: Jul. 15, 2021
(86) PCT No.: PCT/EP2021/069854

§ 371 Date: Jan. 13, 2023

(30) Foreign Application Priority Data
Jul. 16, 2020 (GB) 2010981.5

(51) Int. Cl. C12N 15/86; C12N 15/10

(52) U.S. Cl. CPC C12N 15/86; C12N 15/1086; C12N 2750/14143

(57) ABSTRACT

The invention provides methods of screening for capsid-encoding nucleotide sequences of adeno-associated virus (“AAV”) particles capable of infecting a subject's neurons, or sub-compartments thereof, involving neurons derived from induced pluripotent stem cells or embryonic stem cells. The invention also provides AAV capsids, capsid-encoding nucleotide sequences, expression vectors, viral particles, cells and kits for use in methods of treating neuromuscular or neuromotor disorders, such as spasticity. Sequences generated from the methods may lead to new gene therapies targeted to select populations of neurons, such as motor neurons innervating muscle cells, thus providing high specificity that may be personalised to the subject.

### TECHNICAL FIELD

The present invention relates generally to methods for screening adeno-associated virus (“AAV”) capsids targeting defined populations of neurons, such as motor neurons, which can be used to develop gene therapies for treating neuromuscular and neuromotor disorders, such as spasticity.

### BACKGROUND ART

The symptoms of neurological disorders and diseases result from abnormal function in neurons and circuits in the brain and spinal cord. Current treatments are aimed at targeting this dysfunction and include the relatively new field of functional neurosurgery, using, for example, deep brain stimulation, spinal cord stimulation, or implantation of devices, such as pumps, for local drug delivery. The key to the success of these treatments is the location of the implanted devices in areas of neural dysfunction. In other words, it is critical to treat the dysfunctional neurons.

An alternative to implanting devices is the insertion of genes to modify the function of the neurons—“gene therapy.” But whether implanting devices or inserting genetic material, there is still a problem with specificity: there are many different types of neurons where the devices are implanted, all of which would be affected.

An alternative would be to use gene therapy directed specifically to the neurons involved in the dysfunction of circuits. This could be done by combining three factors: (a) a promoter gene that is specific to the neuron type; (b) insertion of the genetic material at a different location in the circuit, i.e. to where the neurons of interest project, such that this material is transported back to the neuronal cell bodies to modify their function; and (c) enhancing tropism of the virus for the particular neuronal type.

Recombinant adeno-associated vectors (“rAAVs”) are important vectors for the transfer of genetic material into cells, and notably are the predominant viral vector used in gene therapy applications (Li and Sumulski, 2020). Several rAAV-based gene therapies are now in use such as Zolgensma for the treatment of spinal muscular atrophy and Luxtern for the treatment of a subtype if retinal dystrophy, with many more currently being tested in clinical trials.

AAVs are small viruses belonging to the genus dependoparvovirus containing a single strand of DNA, up to ˜4.9 Kb. The AAV genome contains three capsids proteins VP1, VP2 and VP3, all of which are translated from one mRNA. In the wild, multiple serotypes of AAV have been identified each with unique sequences of capsid gene, and accordingly distinct tropisms, although wild serotypes tend to be able to infect multiple tissue and cell types. These serotypes are denoted by numbers: AAV1, AAV2, etc.

Modification of capsid sequences via DNA recombination methods can generate non-native sequences with tailored properties and tropism directed towards (or against) particular cells or tissues, and that evade the immune system (Vandenberghe et al., 2009). In general, modifications to capsids can be achieved in two ways, random mutagenesis of existing capsid DNA sequences, or by capsid shuffling (i.e. taking DNA sequences from multiple capsids and randomly shuffling parts of the sequence to make a new capsid; Buning et al., 2015). These methods generate a large number of highly diverse capsids with potentially valuable properties. When packaged into functional virions, they can be screened in animal tissues or in cell cultures to select capsids that are targeted towards particular tissues or cells. These capsid sequences can then be generated de novo and combined with genes with therapeutic potential in a gene therapy.

This system of screening capsid libraries to generate novel gene therapy vectors has been successfully applied in animals to derivatives of AAV9, which can cross the blood brain barrier and infect neurons after systemic injection. rAAVs such as PhP.eB (reviewed in Bedbrook et al., 2018) were generated in this way. In this system rAAV capsid libraries are injected into experimental animals, and the target cell population (in this case neurons) is then harvested and subjected to sequencing (either by PCR or through deep sequencing methods) to identify sequences of AAV capsids that have infected those cells. These sequences can then be synthesised de novo, and either used in further experiments or subjected to further rounds of mutagenesis and screening (to increase evolutionary pressure), in a process known as directed evolution (see Li and Sumulski, 2020). This type of approach has been used to generate rAAVs targeted towards dopaminergic neurons (Davidsson et al., 2019), striated muscle (Yang et al., 2009) and parts of the retina (Dalkara et al., 2013). rAAV capsids can also be evolved to reduce their infection of vital organs such as the liver (Pulicherla et al., 2011). Finally, directed evolution of rAAV capsids can also be used to target subcompartments of individual cells types. For example, rAAV2-Retro was evolved to target the synaptic terminals of mouse cortical neurons (Tervo et al., 2016).

In vivo studies have shown that directed evolution of AAV capsids can lead to vectors that have useful properties such as an ability to cross the blood brain barrier and increased neurotropism (Deverman et al., 2016 and EP3044318B1), or directed towards dopaminergic neurons (Daviddson et al., 2019) or cardiomyocytes (Yang et al., 2009) amongst others (for review see Li and Sumulski, 2020). Directed evolution involves the generation of a capsid library, a mixture of AAV vectors encapsidated with random capsid sequences generated via error prone PCR, capsid shuffling, or both. This library is then applied to cell lines (e.g. Maheshri et al., 2006), undifferentiated stem cells (Asuri et al., 2012) or, most commonly, in experimental animals (for examples Devermann et al., 2016; Li and Sumulski, 2020; U.S. Pat. No. 8,632,764B2; US20170166926A1; U.S. Pat. No. 9,701,984B2). In addition, there is a preliminary report from one group that used iPSCs differentiated into cardiomyocytes to generate cardiotropic AAVs.

Although the generation of AAV capsid libraries can be achieved in numerous ways, screening of these libraries mostly relies on the use of experimental animals. One major drawback for this method of generating rAAVs that are effective gene therapy vectors is that the capsids identified are often specific to the species or even strain of animal. Thus, many of the improved features and functions of capsids developed in the screening host (i.e. mouse) do not readily transfer to non-human primates and humans (Hordeaux et al., 2018). This could represent a major reason why engineered capsids have yet to broadly replace wild-type variants in clinical and pre-clinical studies (Davidsson et al., 2019).

One example of a neurological disorder or condition that can be treated as described above is spasticity. Spasticity is a neurological symptom suffered by people with a variety of neurological disorders, including but not limited to multiple sclerosis, stroke, traumatic brain injury, spinal cord injury, and cerebral palsy. It is estimated that spasticity affects ˜12 million people around the world, with ˜22% of patients unable to work due to the condition, with almost all patients reporting that their quality of life is affected and ˜50% of patients reporting that their quality of life is greatly impacted. Spasticity results from excessive excitation of muscle by motor neurons, which, because of the disease, become “hyperexcitable.”

Current treatments of spasticity involve oral medications (e.g. baclofen), intramuscular injections of paralysing agents (botulinum toxin), or surgical insertion of pumps to deliver medications (e.g. baclofen) directly to the spinal fluid. Oral medications are often insufficient, or people suffer significant adverse effects. Botox requires repeated administration several times per year for life. Botox is often injected in only a limited number of muscles and may have long term adverse effects. Baclofen pumps can be very effective at alleviating symptoms, however, they are highly invasive as they require surgical intervention for implantation and to be refilled, which happens 2-4 times per year and they must be replaced every 6-7 years. Additionally, in the 6-7 year lifetime of a pump, ⅓ to ½ of patients will need additional surgery to correct a problem. And each operation has a defined complication rate.

Accordingly, there is an urgent need to provide novel gene therapy vectors that are specific for particular neurons, for treating neuromuscular and neuromotor disorders, such as spasticity, and that are capable of being used in subjects such as human subjects.

### DISCLOSURE OF THE INVENTION

The present invention comprises a gene therapy that is designed to alter the activity of targeted neurons. The success of this treatment requires efficient infection (or “transduction”) of neurons, sometimes following intramuscular injection. To the best of the inventors' knowledge, this has not been possible with previous treatments/inventions. In some preferred embodiments, the neurons are motor neurons. In other preferred embodiments, the neurons are sensory neurons, interneurons, or projection neurons.

To achieve this, the invention comprises a technology platform that identifies novel adeno-associated virus (AAV) capsids based on their ability to efficiently infect specific classes of neurons, such as motor neurons. This platform uses stem cells differentiated to these cell types to screen AAV capsid libraries, and use the sequences generated from these screens to develop new gene therapies targeted to select populations of neurons, or other human cells or tissues. The neurons may be defined anatomically, functionally or via gene expression, or by involvement in disease state (such as dopaminergic neurons in Parkinson's disease).

The invention also comprises methods of treatment which involve injecting AAVs comprising the novel AAV capsids into affected muscles of a subject; these AAVs can then infect the terminals of motor neurons, and are transported to their cell bodies leading to the expression of an exogenous transgene specifically in motor neurons innervating that muscle, providing high specificity (see FIG. 1).

The invention can therefore enable the generation of viruses that access motor neurons following intramuscular injection, and subsequently modify gene expression in motor neurons with the goal of curing, alleviating symptoms, and/or improving the quality of life of patients with diseases affecting motor neurons.

The invention also comprises methods of treatment which involve injecting AAVs comprising the novel AAV capsids into the brain or spinal cord of a subject; these AAVs can then infect the terminals of motor neurons, sensory neurons, interneurons or projection neurons of a subject.

The invention can therefore enable the generation of viruses that access these neurons following intracranial or intraspinal intramuscular injection, and subsequently modify gene expression in these neurons with the goal of curing, alleviating symptoms, and/or improving the quality of life of patients with diseases affecting these neurons.

As discussed above, AAV capsids have the potential for exquisite levels of cell and species tropism but currently available screening technologies fail to harness this selectivity and utilise it for therapeutic benefit.

The present invention overcomes these drawbacks and allows for the screening of AAV capsid libraries in human cells derived from induced pluripotent stem cells (“iPSCs”) or embryonic stem cells (“ESCs”). Human cells, including many subtypes of neuron, can be derived in vitro from iPSCs or ESCs (Little et al., 2019). As the genetic makeup of these neurons would be more similar to that of human patients than the same cells found in animal neurons, it is believed that they provide a much more suitable substrate for screening AAV libraries than experimental animals. In some embodiments, the neurons are motor neurons. While motor neurons may be derived from human embryonic neurons, such neurons are of limited supply and are not personalised. In contrast, deriving neurons from iPSCs, as taught by in some aspects of this invention, allows for a greater supply of motor neurons that can also be personalised.

The present invention also comprises a viral evolution approach that uses a combination of neurons derived from induced iPSCs and ESCs, and in vitro screening, to identify AAV capsid-encoding nucleotide sequences that allow for AAV capsids that can more efficiently infect neurons via injection. In some embodiments, this approach allows for the identification of AAV capsid-encoding nucleotide sequences that allow for AAV capsids that can more efficiently infect motor neurons via intramuscular injection.

The invention also comprises a technology platform that identifies novel adeno-associated virus (AAV) capsids based on their ability to target individual compartments (or sub-compartments) of a neuron, by combining iPSC/ESC-derived cell culture with the use of microfluidic chambers. An example use of this is described herein, where AAV capsid libraries can be screened for their ability to efficiently infect motor neuron terminals, but the same system may be used to identify capsids targeted to many neuronal types.

Further, as capsid sequences that efficiently infect neurons may vary from subject to subject, the invention further comprises a process whereby skin samples from individual subjects can be taken, the skin sample transformed into stem cells and then neurons, and these neurons from the patient are used to screen for effective AAV vectors, providing a personalised approach to gene therapy (see FIG. 2).

This invention is advantageous over other treatments as it may require only a single treatment. The screening methods allow for the discovery of novel capsids, so the patient will not be immune to viral particles that comprise them. In the case of motor neurons, it is believed that the rAAVs can be delivered to any muscles (i.e. even difficult to access muscles under anaesthesia in the operating theatre). The capsids may have reduced off target effects and complications, as injections can be applied only to specific symptomatic muscles. At the same time, it will be possible to deliver alternative capsids repeatedly to a patient should that be necessary (e.g. to different muscles) without the development of immune-mediated rejection.

It is believed the invention may also be applied to other motor neuron disorders which currently have little or no effective therapies, as well as to other types of neuron that underlie neurological disorders.

rAAV vectors developed using the methods described herein are especially applicable to the treatment of neuromuscular/neuromotor disorders such as spasticity, amyotrophic lateral sclerosis, dystonia, allowing for the introduction of genetic material into motor neurons via intramuscular injection of viral vectors. For example, disclosed herein is a demonstration of how the methods described herein can form the basis of a gene therapy aimed at alleviating the symptoms of spasticity.

However, the screening methods defined herein are agnostic to the disease being treated and may be used to screen for capsid sequences that effectively target many neuronal types, and as such could be effective in gene therapy approaches regardless of the neuron disorders (or “neuronal disorders”). To the best of the inventors' knowledge, there are currently no methods/inventions described in the literature that suggest screening AAV libraries on cells derived from stem cells, or generating personalised AAV vectors for gene therapy.

Some particular aspects of the invention will now be discussed in more detail.

### Methods of Screening for Capsid-Encoding Nucleotide Sequences of Adeno-Associated Virus (“AAV”) Particles Capable of Infecting Neurons in a Subject

In one aspect, the invention provides a method of screening for capsid-encoding nucleotide sequences of adeno-associated virus (“AAV”) particles capable of infecting neurons in a subject, the method comprising:
(i) providing a population of neurons, wherein said neurons are derived from induced pluripotent stem cells (“iPSCs”) or embryonic stem cells (“ESCs”);
(ii) contacting the population with a first plurality (or “library”) of test AAV particles;
(iii) isolating a first plurality of AAV particles that have infected the neurons; and
(iv) determining the capsid-encoding nucleotide sequences of the first plurality of AAV particles that have infected the neurons.

In another aspect, the invention provides a method of screening for capsid-encoding nucleotide sequences of adeno-associated virus (“AAV”) particles capable of infecting a specific sub-compartment of neurons in a subject, the method comprising:
(i) providing a population of neurons, wherein said neurons are derived from induced pluripotent stem cells (“iPSCs”) or embryonic stem cells (“ESCs”);
(ii) contacting the population with a first plurality (or “library”) of test AAV particles;
(iii) isolating a first plurality of AAV particles that have infected the specific sub-compartment of the neurons; and
(iv) determining the capsid-encoding nucleotide sequences of the first plurality of AAV particles that have infected the neurons.

In another aspect, the invention provides a method of screening for capsid-encoding nucleotide sequences of adeno-associated virus (“AAV”) particles capable of infecting neurons in a subject, the method comprising:
(i) providing a population comprising neurons,
wherein said neurons are derived from induced pluripotent stem cells (“iPSCs”) or embryonic stem cells (“ESCs”), and
wherein said neurons each have a first specific sub-compartment and a second specific sub-compartment;
(ii) disposing said neurons so that the first specific sub-compartments and second specific sub-compartments are distally separated from each other;
(iii) contacting the first specific sub-compartments with a first plurality (or “library”) of test AAV particles;
(iv) isolating a first plurality of AAV particles that have infected the second specific sub-compartments; and
(v) determining the capsid-encoding nucleotide sequences of the first plurality of AAV particles that have infected the second specific sub-compartment of neurons.

In this method, the second specific sub-compartment is the “target” sub-compartment, and in some preferred embodiments it is the cell body of a neuron. In some embodiments, the population of neurons is an enriched population of neurons, whereby over 80%, 81%, 82%, 83%, 84%, 85%, 86%, 87%, 88%, 89%, 90%, 91%, 92%, 93%, 94%, 95%, 96%, 97%, 98%, or 99%, or 100% of the total cells within the population are neurons. In some embodiments, the capsid-encoding nucleotide sequences determined in the screening methods disclose herein may be used for further rounds of directed evolution to increase evolutionary pressure (a viral evolution approach). Accordingly, in some embodiments, the screening methods further comprise, after determining the capsid-encoding nucleotide sequences of the first plurality of AAV particles that have infected the neurons or specific sub-compartment of neurons:
(i) generating a second plurality (or “library”) of test AAV particles using the capsid-encoding nucleotide sequences of the first plurality of AAV particles;
(ii) repeating the steps of the screening methods in order to isolate a second plurality of AAV particles that have infected the neurons or specific sub-compartments of neurons; and
(iii) determining the capsid-encoding nucleotide sequences of the second plurality of AAV particles that have infected the neurons or specific sub-compartments of neurons,
   wherein the capsid-encoding nucleotide sequences of the second plurality of AAV particles are more effective at infecting the neurons or specific sub-compartments of neurons than the capsid-encoding nucleotide sequences of the first plurality of AAV particles.

The effectiveness of an AAV particle to infect neurons or specific sub-compartments of neurons can be determined by counting the number of neurons that express the viral DNA. In some cases, multiple motor neurons innervate the same targets, e.g. muscle in the case of motor neurons, and the proportion of these neurons that have been transduced by the AAV particle can be counted. The effectiveness of an AAV particle to infect neurons or specific sub-compartments of neurons can also be determined by DNA sequencing or RT-PCR to look at the “copy number” of the viral DNA that is in the neural cell. This would give an estimate of how many times the same cell was infected with the AAV particle.

In some embodiments, the second plurality of test AAV particles are generated by one or more of:
i) random mutagenesis of the capsid-encoding nucleotide sequences of the first plurality of AAV particles;
ii) shuffling of the capsid-encoding nucleotide sequences of the first plurality of AAV particles; and
iii) insertion of targeted or random peptide sequences up to 25 amino acids in length at various regions in VP1, VP2 or VP3 of the capsid-encoding nucleotide sequences of the first plurality of AAV particles.

In some cases, the sub-compartment of neurons is a neuronal cell body, neurite, axon or dendrite. In some cases, the sub-compartment of neurons is the axon terminal (or “synaptic terminal”). In some cases, the second sub-compartment is a neuronal cell body. In some cases in step (iii) above, targeted or random peptides sequences of 1, 2, 3, 4, 5, 6, 7, 8, 9, 10, 11, 12, 13, 14, 15, 16, 17, 18, 19, 20, 21, 22, 23, 24 or 25 amino acids are inserted at various regions in VP1, VP2 or VP3 of the capsid-encoding nucleotide sequences of the first plurality of AAV particles. In some cases these additional steps may be repeated 1, 2, 3, 4, 5, 6, 7, 8, 9, or 10 times.

In aspects of the invention where sub-compartments of neurons are distally separated from each other, the neurons are cultured in such a way that allows for the separation of neuronal cell bodies and neurites such that the rAAV library can be applied to one and not the other (such as with the use of microfluidic chambers to separate axonal projections and synaptic terminals from neuronal soma for the purposes of library screening).

In aspects of the invention where sub-compartments of neurons are distally separated from each other, the first specific sub-compartments and second specific sub-compartments may be grouped in different physical regions of one or more containers. The first specific sub-compartments and second specific sub-compartments may be connected by axons. In some cases, the first specific sub-compartment is a neural synaptic terminal, and the second specific sub-compartment is a neural cell body.

In aspects of the invention where sub-compartments of neurons are distally separated from each other, the first specific sub-compartments and second specific sub-compartments may be grouped in different physical regions of one or more containers in a microfluidic chamber. The one or more containers may further be separated by microfluidic channels. The neurons may be grown further once added to the microfluidic chamber. The microfluidic chamber may be an AXIS™ Axon Isolation Device.

The application of AAV capsid libraries to one component of a neuron (such as the axon, dendrite, neurite, or axon terminal (or “synaptic terminal”)) and harvesting genetic material from another region of the cells (such as the cell body) allows for the identification and determination of AAV capsid sequences that have successfully infected those cells via that specific route.

There are several variations of library that can be used as a starting point, which can be assessed by determining the genetic sequences from the AAVs and comparing them to AAVs of different libraries. Further, the time between i) exposing neurons to the libraries of AAVs and ii) harvesting the neurons in order to isolate the AAVs that have infected the neurons, (the “incubation time”) can be optimised.

In some embodiments, multiple types of cells derived from iPSCs or ESCs may be added to the population of neurons, in order to evolve AAV capsids towards or against infection of one or more of those cell types. Harvesting genetic material from all or some of these cells, either individually (for example, only motor neurons) or in combination (for example, motor neurons and sensory neurons) allows for the determination of capsid sequences that have successfully infected these cells.

In some embodiments, such additional cells may be skeletal muscle cells, myocytes, or sensory neurons. The additional cell may be a myotube. The additional cell may also be a myoblast or muscle fiber. The additional cell may be a C2C12 cell.

In some cases, the iPSCs or ESCs are derived from the subject. In some cases, the iPSCs or ESCs are derived from a skin sample of the subject. In some cases, the iPSCs or ESCs are derived from fibroblasts of the subject. In some cases, the subject is a human subject. The iPSCs and ECSs may also be obtained from an animal, a human subject/patient, or from a cell bank. The screening of AAV capsid libraries using iPSCs from a human subject or patient allows for the identification of capsid sequences that infect neurons or other cells from that subject or patient. Accordingly, the capsid sequences can be generated on an individualised basis using this method.

In some cases, step (i) comprises a step of deriving neurons from i) iPSCs or ii) ESCs.

In some cases, the method is a method a screening for capsid-encoding nucleotide sequences of adeno-associated virus (“AAV”) particles capable of infecting neurons in a subject via intramuscular injection.

In some cases, the neurons are derived from iPSCs. In some cases, the neurons are derived from ESCs. In some cases, human iPSCs can be easier to retrieve than ESCs.

As used herein, the term “neuron” includes a neuron and a portion or portions thereof (e.g., the neuron cell body, an axon, or a dendrite). The term “neuron” as used herein denotes nervous system cells that include a central cell body or soma, and two types of extensions or projections: dendrites, by which, in general, the majority of neuronal signals are conveyed to the cell body, and axons, by which, in general, the majority of neuronal signals are conveyed from the cell body to effector cells, such as target neurons or muscle. Neurons can convey information from tissues and organs into the central nervous system (afferent or sensory neurons) and transmit signals from the central nervous systems to effector cells (efferent or motor neurons). Other neurons, designated interneurons, connect neurons within the central nervous system (the brain and spinal cord). Other neurons, designated project (or “projection”) neurons, extend their axons from one region of the nervous system to another. The screening methods disclosed herein may be used to screen for capsid-encoding nucleotide sequences of adeno-associated virus (“AAV”) particles capable of infecting such neurons. In some preferred embodiments, the neurons are motor neurons. In other preferred embodiments, the neurons are sensory neurons, interneurons, or projection neurons.

Further, as mentioned above, it is believed that the screening methods described above may be applied to screen for capsid-encoding nucleotide sequences of AAV particles capable of infecting other cell types such as sensory neurons, basal ganglia projection neurons, dopaminergic neurons, and muscle tissue.

In some cases, the screening methods described herein comprise a step of providing a plurality of test AAV particles that have been additionally screened for capability of infecting muscle cells, for example by direct intramuscular injection. Said additional screening may occur before, after or concurrently with screening of the particles for capability of infecting neurons.

The methods described herein may be in vivo or in vitro.

Culturing of Fibroblasts from Human Skin Biopsies

In some embodiments, the iPSCs used in the screening methods described herein are derived from a human test subject. In some embodiments, the iPSCs are derived from a culture of fibroblasts from skin biopsies of the subject.

Some methods that allow for culturing of fibroblasts from skin biopsies have been previously described (Vangipuram M, Ting D, Kim S, Diaz R, Schüle B. Skin punch biopsy explant culture for derivation of primary human fibroblasts. J Vis Exp. 2013; (77):e3779. Published 2013 Jul. 7. doi:10.3791/3779).

In some preferred embodiments, the method comprises one or more of the following steps:
Under sterile conditions a skin biopsy is placed in a gelatin coated culture dish with Dulbecco's modified Eagle medium (DMEM) containing 20% foetal bovine serum (FBS).
Under a dissecting microscope the biopsy is cut into pieces around 200-500 μm in size. 2-3 pieces of the biopsy are transferred to a fresh gelatin coated culture dish containing DMEM/20% FBS.
Media is replaced every 2 days until fibroblasts are confluent. The cells can then be trypsinised and transferred to a new culture vessel for expansion. Cells can either be frozen in liquid nitrogen or used for derivation into IPSCs.
   Generating IPSCs from Fibroblasts

In some embodiments, the iPSCs used in the screening methods described herein are derived from a human test subject. In some embodiments, the iPSCs are derived from a culture of fibroblasts from skin biopsies of the subject.

Derivation of human fibroblasts into IPSCs can be achieved using a commercial kit such as the CytoTune-IPS Sendai reprogramming kit from ThermoFisher. Further details may be found at https://www.thermofisher.com/order/catalog/product/A16517#/A16517 and https://assets.thermofisher.com/TFS-Assets/LSG/manuals/cytotune_ips_2_0_sendai_reproq_kit_man.pdf.

This uses Sendai virus to introduce Yamanaka factors (Oct3/4, Sox2, Klf4, c-Myc) into somatic cell types. These factors are highly expressed in embryonic stem cells and overexpression induces pluripotency in human and mouse somatic cells (see Takahashi, K., Yamanaka, S). A decade of transcription factor-mediated reprogramming to pluripotency. Nat Rev Mol Cell Biol 17, 183-193 (2016). https://doi.org/10.1038/nrm.2016.8). Several methods are available that allow for the generation of IPSCs from fibroblasts, which may rely on the introduction of some combination of these factors into fibroblasts.

In some preferred embodiments, methods for generating IPSCs from fibroblasts comprise one or more of the following steps:
Fibroblasts are plated to 30-60% confluency.
Transductions are completed at the following a multiplicity of infection (MOI) for each factor KOS MOI=5, hc-Myc MOI=5, hKlf4 MOI=3).
The appropriate volume of each Sendai virus is applied to the fibroblasts (Day 0) and cells are incubated overnight at 37 degrees C./5% CO2.
Media is replaced the following day (Day 1) and then every 2 days until Day 7.
On Day 7, Cells are trypsinised, counted and replated on vitronectin coated 6-well plates at a density of ˜2×10\hat{ }5 cells per well.
On Day 8 media is changed to Essential 8 media and replaced every day thereafter. 3-4 weeks after initial transduction IPSO colonies should have grown to an appropriate size.
IPSO colonies are harvested manually or through the use of chemical dissociation, stored in liquid nitrogen or used directly for derivation into primary cell types.
iPSCs are maintained on Geltrex (Life Technologies) with Essential 8 Medium media (Life Technologies), and passaged using EDTA (Life Technologies, 0.5 mM) at 37° C. and 5% carbon dioxide. In some cases, the use of Geltrex can increase cell viability, when compared to other substrates. In some cases, the use of EDTA and Essential 8 media is particularly useful for generating human IPSCs, with EDTA increasing cell survival by about 20% compared to other dissociation methods
   Differentiation of iPSCs into Limb Innervating Motor Neurons

In some embodiments, the iPSCs and ESCs used in the methods are differentiated into somatic motor neurons.

Some methods that allow for differentiation of iPSCs or ESCs into somatic motor neurons have been previously described in the following:

Wichterle, H. and Peljto, M. (2008), Differentiation of Mouse Embryonic Stem Cells to Spinal Motor Neurons. Current Protocols in Stem Cell Biology, 5: 1H.1.1-1H.1.9. doi:10.1002/9780470151808.sc01h01s5;

Journal of Neuroscience 8 Sep. 2004, 24 (36) 7848-7858; DOI: 10.1523/JNEUROSCI.1972-04.2004; and

Amoroso M W, Croft G F, Williams D J, et al. Accelerated high-yield generation of limb-innervating motor neurons from human stem cells. J Neurosci. 2013; 33(2):574-586. doi:10.1523/JNEUROSCI.0906-12.2013.

In some preferred embodiments, methods that allow for differentiation of iPSCs into limb innervating motor neurons include one or more of the following steps:
IPSCs are maintained in media consisting of DMEM: nutrient mixture F-12 (DMEM/F:12; Invitrogen) with 20% Knockout Serum Replacer (Invitrogen), 110 μM β-mercaptoethanol( ), I-glutamine and nonessential amino acids (NEAA; Invitrogen), and 20 ng/ml basic fibroblast growth factor (bFGF; Invitrogen) in suspension, supplemented with 10 μM Rho-associated kinase inhibitor Y27632 (Ascent Scientific) to enhance single cell survival, 20 ng/ml bFGF (Invitrogen) to enhance growth, and 10 μm SB435142 (SB; Sigma) and 0.2 μM LDN193189 (LDN; Stemgent) for neuralization, Neurobasal, N2 supplement, B27 supplement, and insulin. Cells are passaged using dispase. In some cases, the use of Neurobasal, N2 supplement, B27 supplement, insulin and/or dipase leads to improved cell viability.
At day 0 embryoid bodies (EBs) are switched to neural induction medium (DMEM/F:12 with I-glutamine; NEAA; penicillin/streptomycin; heparin, 2 μg/ml; N2 supplement; Invitrogen). and 1 μM Dorsomorphin (Millipore), 2 μM SB431542 (Tocris Bioscience), and 3 μM CHIR99021 (Miltenyi Biotec).Two days later all-trans retinoic acid (RA; 1 μm; Sigma), ascorbic acid (0.4 μg/ml; Sigma), and brain-derived neurotrophic factor (10 ng/ml; R&D) are added. In some cases, the use of 1 μM Dorsomorphin, 2 μM SB431542, and/or 3 μM CHIR99021 leads to improved cell viability.
At day 8, the cultures are enzymatically dissociated using dispase (GIBCO, 1 mg/ml), plated onto laminin coated plates and next patterned for 7 days with 1 μM retinoic acid (Sigma), ascorbic acid (0.4 μg/ml; Sigma), and brain-derived neurotrophic factor (10 ng/ml; R&D). In some cases, this step leads to improved cell viability.
At day 14 spinal cord MN precursors were treated with 0.1 μM Purmorphamine for a further 4 days. In some cases, this step leads to improved cell viability.
At day 18 basal medium is changed to Neurobasal (Invitrogen), containing all previous factors and with the addition of 10 ng/ml each of insulin-like growth factor 1 (IGF-1), glial cell line-derived neurotrophic factor (GDNF), and ciliary neurotrophic factor (CNTF) (R&D), plus B27 (Invitrogen) and 0.1 μM Compound E (Enzo Life Sciences). In some cases, the use of 0.1 μM Compound E leads to improved cell viability.
After a further ˜5 days, EBs are dissociated with 0.05% trypsin (Invitrogen) and plated onto poly-lysine/laminin-coated coverslips or directly into the motor neuron compartment of microfluidic chambers (see FIG. 3) for use in AAV screening.
   Differentiation of iPSCs into Skeletal Muscle Fibres

In some preferred embodiments, iPSCs are also derived into skeletal muscle cells (or “skeletal muscle fibres”), which may be added to the populations of motor neurons used in the screening methods described herein.

Some methods that allow for differentiation of iPSCs into muscle fibres are previously been described in the following:

Maffioletti, S., Gerli, M., Ragazzi, M. et al. Efficient derivation and inducible differentiation of expandable skeletal myogenic cells from human ES and patient-specific iPS cells. Nat Protoc 10, 941-958 (2015). https://doi.org/10.1038/nprot.2015.057.

In some preferred embodiments, methods that allow for differentiation of iPSCs into muscle fibres include on or more of the following steps:
Dissociated IPSO colonies are cultured on Matrigel-coated dishes in Minimal Essential Media alpha containing 10% foetal bovine serum, 2 mM Glutamine, 0.1 mM 2-Mercaptoethanol
Media is replaced every day for one week
Cells are detached from the Matrigel when 100% confluent using trypsin and re-plated on plastic at a density of 1.2×10\hat{ }4 cells per cm².
Cells can be expanded and cryopreserved for future use or used for myogenic differentiation.
For derivation, cells are plated in 35 mm dishes in media comprised of MegaCell DMEM containing 5% foetal bovine serum, 2 mM glutamine, 1% non-essential amino acids, 50 μM 2-Mercaptoethanol, 5 ng/ml basic fibroblast growth factor at a density of ˜1×10\hat{ }5.
Cultures are infected with a lentivirus expressing myoblast determination protein 1 (MDP1) at a multiplicity of infection of 1, 10 and 50 in the presence of polybrene.
Cells are inspected daily and cultures expanded as required.
Myotubes are usually identifiable after ˜3 days of the expression of MDP1. Some of the cultures can be fixed and tested for the expression of myosin heavy chain.
Remaining cultures are optionally re-plated into the microfluidic chambers, in the muscle compartment (see FIG. 3). These myocytes are usually cultured into the muscle compartment ˜5-7 days after the addition of the motor neurons, by which time motor neuron axons should have begun to enter the muscle compartment.
Cultures are optionally kept for ˜1-3 weeks to allow addition of the AAV capsid library.

### Production of AAV Libraries and Application of AAV Capsid Libraries to Neurons in Microfluidic Chambers

Diverse capsid libraries can be generated through a process of i) random mutagenesis of naturally occurring capsids, ii) shuffling of naturally occurring capsids, iii) insertion of targeted or random peptide sequences up to 25 amino acids in length at various regions in VP1, VP2 or VP3 of the AAV capsid or iv) a combination of the above.

To generate the library the randomised capsid sequences are cloned into an AAV backbone containing the AAV2 inverted terminal repeats (ITRs; packaging signals) and the AAV rep gene. These DNA plasmids are transfected in to HEK293 in the presence of additional adenoviral genes to facilitate AAV packaging. AAV virions are harvested from HEK293 cells and/or the culture medium, purified and concentrated following standard methods (e.g. Potter et al., 2014 https://dx.doi.org/10.1038%2Fmtm.2014.34; McClure et al., 2011 http://dx.doi.org/10.3791/3348)

In some embodiments, methods for production of AAV libraries comprise one more of the following steps:
Purified and concentrated AAV libraries are diluted in Dulbecco's modified Eagle medium (DMEM) and this is applied to the muscle chamber of a microfluidic device.
2-10 days after application neuronal cell bodies are harvested either by chemical (i.e. trypsinisation) or mechanical (cell scraping) methods.
The neurons are lysed and the lysate can either be submitted for deep sequencing (such as RNAseq) to directly detect capsid sequences found in the neurons, or the lysate can be used as a PCR template using primers directed against conserved regions of the AAV capsid. Following PCR of capsid regions the DNA fragment is cloned into a DNA vector and submitted for Sanger sequencing.
Capsid sequences harvested from neurons are analysed using bioinformatics for conserved regions and highly enriched capsids can be de novo synthesised and can then undergo either further mutagenesis or repeat of in vitro screening to increase evolutionary pressure through directed evolution.
Directed evolution can be repeated for several rounds (˜2-5 rounds). Capsid sequences that show efficient retrograde transport in vitro can be used to generate functional virions for in vivo use in animals or humans.
   rAAV Vectors Comprising Capsid Sequences that can Lead to Infection of Neurons In one aspect, the invention provides an AAV capsid identified by a screening method of the invention. In another aspect, the invention provides an AAV capsid-encoding nucleotide sequence having at least 50, 55, 60, 65, 70, 75, 80, 85, 90, 91, 92, 93, 94, 95, 96, 97, 98, 99, or 100% sequence identity to SEQ ID NO: 1 or SEQ ID NO:2. In another aspect, the invention provides an AAV capsid-encoding nucleotide sequence having at least 50, 55, 60, 65, 70, 75, 80, 85, 90, 91, 92, 93, 94, 95, 96, 97, 98, 99, or 100% sequence identity to SEQ ID NO: 11. In another aspect, the invention provides an AAV capsid-encoding nucleotide sequence having at least 50, 55, 60, 65, 70, 75, 80, 85, 90, 91, 92, 93, 94, 95, 96, 97, 98, 99, or 100% sequence identity to SEQ ID NO: 13.

In some cases, a nucleotide sequence disclosed herein differs from a wild-type AAV vector by 1, 2, 3, 4, 5, 6, 7, 8, 9, 10, 15, 20, 25, 30, 35, 40, 45, 50, 60, 70, 80, 90 or 100 base pairs.

In some cases, a nucleotide sequence disclosed herein differs from SEQ ID NO: 13 by 1, 2, 3, 4, 5, 6, 7, 8, 9, 10, 15, 20, 25, 30, 35, 40, 45, 50, 60, 70, 80, 90 or 100 base pairs. In some cases, a nucleotide sequence disclosed herein differs from SEQ ID NO: 13 by 1-10, 2-8 or 4-6 base pairs.

Said nucleotides may be used to develop gene therapies involving viruses that access neurons following intramuscular injection, and subsequently modify activity and/or gene expression in neurons. Said nucleotides may also be used to develop gene therapies involving viruses that access neurons following intramuscular injection, and subsequently modify activity and/or gene expression in neurons.

Accordingly, in another aspect, the invention provides an adeno-associated virus (“AAV”) expression vector comprising a capsid nucleotide sequence of an AAV capsid identified by a screening method of the invention. In another aspect, the invention provides a recombinant adeno-associated virus (“AAV”) expression vector comprising a capsid-encoding nucleotide sequence having at least 50, 55, 60, 65, 70, 75, 80, 85, 90, 91, 92, 93, 94, 95, 96, 97, 98, 99, or 100% sequence identity to SEQ ID NO: 1 or SEQ ID NO: 2. In another aspect, the invention provides a recombinant adeno-associated virus (“AAV”) expression vector comprising a capsid-encoding nucleotide sequence having at least 50, 55, 60, 65, 70, 75, 80, 85, 90, 91, 92, 93, 94, 95, 96, 97, 98, 99, or 100% sequence identity to SEQ ID NO: 11. In another aspect, the invention provides a recombinant adeno-associated virus (“AAV”) expression vector comprising a capsid-encoding nucleotide sequence having at least 50, 55, 60, 65, 70, 75, 80, 85, 90, 91, 92, 93, 94, 95, 96, 97, 98, 99, or 100% sequence identity to SEQ ID NO: 13.

In some embodiments, the expression vector is capable of altering the activity of targeted motor neurons in a subject. In some embodiments, the expression vector is capable of altering the activity of targeted motor neurons in a subject via intramuscular injection. In some cases, the expression vector may further comprise a second capsid-encoding nucleotide sequence that is capable of preventing an immune response being invoked in a subject.

### Expression Vectors

An expression vector as used herein is a DNA molecule used to transfer and express foreign genetic material in a cell. Such vectors include a promoter sequence operably linked to the gene encoding the protein to be expressed. “Promoter” means a minimal DNA sequence sufficient to direct transcription of a DNA sequence to which it is operably linked. “Promoter” is also meant to encompass those promoter elements sufficient for promoter-dependent gene expression controllable for cell type specific expression; such elements may be located in the 5′ or 3′ regions of the native gene. Alternatively an expression vector may be an RNA molecule that undergoes reverse transcription to DNA as a result of the reverse transcriptase enzyme.

An expression vector may also include a termination codon and expression enhancers. Any suitable vectors, enhancers and termination codons may be used to express the gene product, such as a Kv1 potassium channel, from an expression vector according to the invention. Expression vectors include viral vectors such as AAV vectors.

Generally speaking, those skilled in the art are well able to construct vectors and design protocols for recombinant gene expression. Suitable vectors can be chosen or constructed, containing, in addition to the elements of the invention described above, appropriate regulatory sequences, including promoter sequences, terminator fragments, polyadenylation sequences, marker genes and other sequences as appropriate. Molecular biology techniques suitable for the expression of polypeptides in cells are well known in the art. For further details see, for example, Molecular Cloning: a Laboratory Manual: 2nd edition, Sambrook et al, 1989, Cold Spring Harbor Laboratory Press or Current Protocols in Molecular Biology, Second Edition, Ausubel et al. eds., John Wiley & Sons, (1995, and periodic supplements).

The term “operably linked” used herein includes the situation where a selected gene and promoter are covalently linked in such a way as to place the expression of the gene (i.e. polypeptide coding) under the influence or control of the promoter. Thus, a promoter is operably linked to a gene if the promoter is capable of effecting transcription of the gene into RNA in a cell. Where appropriate, the resulting RNA transcript may then be translated into a desired protein or polypeptide. The promoter is suitable to effect expression of the operably linked gene in a mammalian cell. Preferably, the mammalian cell is a human cell.

### AAV Vectors

The vector is a recombinant AAV vector. AAV vectors are DNA viruses of relatively small size that can integrate, in a stable and site-specific manner, into the genome of the cells that they infect. They are able to infect a wide spectrum of cells without inducing significant effects on cellular growth, morphology or differentiation. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

AAV vectors may be prepared using standard methods in the art. Adeno-associated viruses of any serotype are suitable (see, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” in Parvoviruses (J R Kerr, S F Cotmore. M E Bloom, R M Linden, C R Parrish, Eds.) p 5-14, Hudder Arnold, London, U K (2006); and D E Bowles, J E Rabinowitz, R J Samulski “The Genus Dependovirus” (J R Kerr, S F Cotmore. M E Bloom, R M Linden, C R Parrish, Eds.) p 15-23, Hudder Arnold, London, UK (2006), the disclosures of which are hereby incorporated by reference herein in their entireties). Methods for purifying for vectors may be found in, for example, U.S. Pat. Nos. 6,566,118, 6,989,264, and 6995006 and International Patent Application Publication No.: WO/1999/011764 titled “Methods for Generating High Titer Helper-free Preparation of Recombinant AAV Vectors”, the disclosures of which are herein incorporated by reference in their entirety.

Preparation of hybrid vectors is described in, for example, PCT Application No. PCT/US2005/027091, the disclosure of which is herein incorporated by reference in its entirety. The use of vectors derived from the AAVs for transferring genes in vitro and in vivo has been described (See e.g., International Patent Application Publication Nos: WO 1/18088 and WO 93/09239; U.S. Pat. Nos. 4,797,368, 6,596,535, and 5,139,941; and European Patent No: 0488528, all of which are herein incorporated by reference in their entirety). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs according to the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In some instances, useful AAV vectors for the expression constructs as described herein include those encapsidated into a virus particle (e.g. AAV virus particle including, but not limited to, AAV1, AAV2, AAV3, AAV4, AAV5, AAV6, AAV7, AAV8, AAV9, AAV10, AAV11, AAV12, AAV13, AAV14, AAV15, and AAV16). Accordingly, the instant disclosure includes a recombinant virus particle (recombinant because it contains a recombinant polynucleotide) comprising any of the vectors described herein. In some preferred embodiments, the AAV virus particle is AAV2. In some preferred embodiments, the AAV virus particle is AAV6. Using AAV6 may lead to improved efficacy of motor neuron infection after intramuscular injection.

In some embodiments, the viral vector contains a sequence encoding a reporter protein, such as a fluorescent protein. In other embodiments the viral vector lacks a sequence encoding a reporter protein, such as a fluorescent protein.

In some embodiments, the viral vector additionally comprises genes encoding viral packaging and envelope proteins.

The expression vector may further comprise a rep gene, optionally wherein the rep gene is an AAV2 rep gene. The expression vector may further comprise a cap gene, optionally wherein the cap gene is an AAV2 cap gene. The expression vector may further comprise an inverted terminal repeat, optionally wherein the inverted terminal repeat is an AAV2 inverted terminal repeat. The expression vector may further comprise a gene encoding a viral packaging and/or envelope protein. The expression vector may further comprise a regulatory gene, optionally wherein the regulatory gene is polyA.

### AAV Vectors Further Comprising Transgenes

In some embodiments, the expression vector further comprises a transgene encoding a transgene product, wherein the transgene product is capable of altering the activity of targeted motor neurons in a subject. In some embodiments, the transgene product is capable of altering the activity of targeted motor neurons in a subject via intramuscular injection.

In some embodiments, the gene product is capable of altering the excitability of neurons when the expression vector is administered to a subject. In some embodiments, the gene product is capable of reducing the hyperexcitability of neurons when the expression vector is administered to a subject. The transgene to be carried by the expression vector depends on the patient's symptoms. For example, in the case of spasticity, the payload can be aimed at alleviating the hyperexcitability of motor neurons. In some embodiments, the transgene expressed by the virus will change the electrical properties of neurons. This may include the use of specific promoters to initially drive one of several different transgenes: those that simply dampen neuron excitability (e.g. KCC2, Kv1) or block synaptic transmission completely (e.g. tetanus toxin light chain), or those that are responsive to low dose medication that the patient takes by mouth so that symptoms can be managed by dose adjustments (e.g. the DREADD hM4Di, that is response to e.g. clozapine PSAM4-GlyR, that is responsive to e.g. uPSEM, or varenicline). To the best of the inventors' knowledge, there are currently no methods described in the literature suggesting the combination of intramuscular gene delivery and altering neuronal excitability to treat spasticity.

The DREADD hM4Di is a mutated muscarinic acetylcholine receptor that can bind synthetic ligands such as clozapine-N-oxide, resulting in silencing of neuronal activity. KCC2 is a potassium-chloride transporter found in neurons that can extrude chloride ions from the cell, which control the excitability of the neuron. Tetanus toxin light chain is part of the tetanus toxin neurotoxin that can specifically cleave a protein (VAMP2) on synaptic vesicles. Cleavage of VAMP2 prevents synaptic vesicle docking and neurotransmitter release. Potassium channels, such as Kv1, can specifically reduce the excitability of neurons. In some cases, the ability of the gene product to alter the excitability of motor neurons can be determined by electrophysiology recordings (patch clamp) on the infected cells. The responses (frequency of action potentials) of the infected neurons to injected currents, measured as the slope(s) of their frequency/current (f/I) curves, can be recorded in order to determine their excitability. This can involve dose-response curves for the ligands listed above.

The transgene product may be capable of dampening neuron excitability when the expression vector is administered to a subject. In some embodiments, the transgene or transgene product is a KCC2 transgene or transgene product. Potassium-chloride transporter member 5 (KCC2) a neuron-specific chloride potassium symporter responsible for establishing the chloride ion gradient in neurons through the maintenance of low intracellular chloride concentrations. Animals with reduced expression of this transporter exhibit severe motor deficits, epileptiform activity, and spasticity. In some embodiments, the KCC2 transgene has at least 50, 55, 60, 65, 70, 75, 80, 85, 90, 91, 92, 93, 94, 95, 96, 97, 98, 99, or 100% sequence identity to SEQ ID NO: 3; or the KCC2 transgene product has at least 50, 55, 60, 65, 70, 75, 80, 85, 90, 91, 92, 93, 94, 95, 96, 97, 98, 99, or 100% sequence identity to SEQ ID NO: 4.

In some embodiments, the transgene or transgene product is a Kv1 transgene or transgene product. The Kv1 potassium channel is a voltage-gated delayed-rectifier potassium channel that is phylogenetically related to the Drosophila Shaker channel. Voltage-dependent potassium channels modulate excitability by opening and closing a potassium-selective pore in response to voltage. In many cases, potassium ion flow can be interrupted when an intracellular particle occludes the pore, a process known as fast inactivation. Kv1 potassium channel subunits have six putative transmembrane segments, and the loop between the fifth and sixth segment of each of the four Kv1 subunits that make up a complete channel forms the pore. In some embodiments, the Kv1 transgene has at least 50, 55, 60, 65, 70, 75, 80, 85, 90, 91, 92, 93, 94, 95, 96, 97, 98, 99, or 100% sequence identity to SEQ ID NO: 5; or the Kv1 transgene product has at least 50, 55, 60, 65, 70, 75, 80, 85, 90, 91, 92, 93, 94, 95, 96, 97, 98, 99, or 100% sequence identity to SEQ ID NO: 6.

The transgene product may be capable of blocking synaptic transmission of neurons when the expression vector is administered to a subject. In some embodiments, the transgene or transgene product is a tetanus toxin light chain transgene or transgene product. In some embodiments, the tetanus toxin light chain transgene has at least 50, 55, 60, 65, 70, 75, 80, 85, 90, 91, 92, 93, 94, 95, 96, 97, 98, 99, or 100% sequence identity to SEQ ID NO: 7; or the tetanus toxin light chain transgene product has at least 50, 55, 60, 65, 70, 75, 80, 85, 90, 91, 92, 93, 94, 95, 96, 97, 98, 99, or 100% sequence identity to SEQ ID NO: 8.

The transgene product may be a receptor activated solely by a synthetic ligand (RASSL) or designer receptor exclusively activated by designer drugs (DREADD). RASSLs and DREADDs are a class of chemogenetically-engineered proteins that permit spatial and temporal control of G protein signalling in vivo. In some embodiments, the transgene or transgene product is an hM4Di transgene or transgene product. In some embodiments, the hM4Di transgene has at least 50, 55, 60, 65, 70, 75, 80, 85, 90, 91, 92, 93, 94, 95, 96, 97, 98, 99, or 100% sequence identity to SEQ ID NO: 7; or the hM4Di transgene product has at least 50, 55, 60, 65, 70, 75, 80, 85, 90, 91, 92, 93, 94, 95, 96, 97, 98, 99, or 100% sequence identity to SEQ ID NO: 8.

In some preferred embodiments, the transgene is operably linked to a neuron-specific promoter.

Alignment and calculation of percentage amino acid or nucleotide sequence identity can be achieved in various ways known to a person of skill in the art, for example, using publically available computer software such as ClustalW 1.82, T-coffee or Megalign (DNASTAR) software. When using such software, the default parameters, e.g. for gap penalty and extension penalty, are preferably used. The default parameters of ClustalW 1.82 are: Protein Gap Open Penalty=10.0, Protein Gap Extension Penalty=0.2, Protein matrix=Gonnet, Protein/DNA ENDGAP=−1, Protein/DNA GAPDIST=4.

The percentage identity can then be calculated from the multiple alignment as (N/T)*100, where N is the number of positions at which the two sequences share an identical residue, and T is the total number of positions compared. Alternatively, percentage identity can be calculated as (N/S)*100 where S is the length of the shorter sequence being compared. The amino acid/polypeptide/nucleic acid sequences may be synthesised de novo, or may be native amino acid/polypeptide/nucleic acid sequence, or a derivative thereof.

Due to the degeneracy of the genetic code, it is clear that any nucleic acid sequence could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid.

### Addition of Transgene Products to Alter Neuron Activity

For the testing of various AAV transgene products (or “cargoes” or “payloads”) that can be used to alter neuron activity, genes can be cloned into an AAV backbone containing the AAV inverted terminal repeats (ITRs) along with either a general or cell-type targeted promoter and common regulatory elements (such as the Woodchuck post translational regulatory element and polyA).

The gene that is used will depend on the need of the patient, and may belong to one of the 2 following classes: (a) ligand-independent, in which the excitability is dampened continually, or (b) ligand-dependent, in which the neuron excitability can be regulated by taking an approved medication.

Within each category, several genes may be tested on that patient's neurons using electrophysiological techniques to determine the best selection for that patient.

In some preferred embodiments, methods for the addition of cargo to alter neuron activity may include one or more of the following steps:
Capsid sequences identified in the screening methods described herein are de novo synthesised and inserted into an AAV helper plasmid containing the AAV2 REP gene (Rep/Cap).
The Rep/Cap plasmid is combined with the AAV backbone and additional plasmids containing adeno-viral helper genes (such as pHelper) and transiently transfected into HEK293 cells.
AAV particles are purified using standard methods and can be used for experiments in vitro or in vivo (e.g. Potter et al., 2014 https://dx.doi.org/10.1038%2Fmtm.2014.34; McClure et al., 2011 http://dx.doi.orq/10.3791/3348).

### Viral Particles

The invention also includes in vitro methods of making AAV viral particles. In one embodiment, this method involves transducing mammalian cells with a viral vector or expression vector as described herein and expressing viral packaging and envelope proteins necessary for particle formation in the cells and culturing the transduced cells in a culture medium, such that the cells produce viral particles that are released into the medium. An example of a suitable mammalian cell is a human embryonic kidney (HEK) 293 cell.

It is possible to use a single expression vector that encodes all the viral components required for viral particle formation and function. Most often, however, multiple plasmid expression vectors or individual expression cassettes integrated stably into a host cell are utilised to separate the various genetic components that generate the viral vector particles.

In some embodiments, expression cassettes encoding the one or more viral packaging and envelope proteins have been integrated stably into a mammalian cell. In these embodiments, transducing these cells with a viral vector described herein is sufficient to result in the production of viral particles without the addition of further expression vectors.

In other embodiments, the in vitro methods involve using multiple expression vectors. In some embodiments, the method comprises transducing the mammalian cells with one or more expression vectors encoding the viral packaging and envelope proteins that encode the viral packaging and envelope proteins necessary for particle formation.

The ssDNA AAV genome contains two open reading frames, Rep and Cap, flanked by two 145 base inverted terminal repeats (ITRs) fundamental for the synthesis of the complementary DNA strand. Rep and Cap produce multiple proteins (Rep78, Rep68, Rep52, Rep40, which are required for the AAV life cycle; and VP1, VP2, VP3, which are capsid proteins). The transgene will be inserted between the ITRs and Rep and Cap in trans. An AAV2 backbone is commonly used and is described in Srivastava et al., J. Virol., 45: 555-564 (1983). Cis-acting sequences directing viral DNA replication (ori), packaging (pkg) and host cell chromosome integration (int) are contained within the ITRs. AAVs also require a helper plasmid containing genes from adenovirus. These genes (E4, E2a and VA) mediate AAV replication. An example of a pAAV plasmid is available from Addgene (Cambridge, MA, USA) as plasmid number 112865 or 60958.

Following release of viral particles, the culture medium comprising the viral particles may be collected and, optionally the viral particles may be separated from the culture medium. Optionally, the viral particles may be concentrated.

Following production and optional concentration, the viral particles may be stored, for example by freezing at −80° C. ready for use by administering to a cell and/or use in therapy.

The invention also provides viral particles, for example those produced by the methods described herein. As used herein, a viral particle comprises a DNA or RNA genome packaged within the viral envelope that is capable of infecting a cell, e.g. a mammalian cell. A viral particle may be integrase deficient, e.g. it may contain a mutant integrase enzyme or contain alterations in the 5′ and/or 3′ LTRs as described herein.

In another aspect, the invention provides a capsid encoded by a capsid-encoding nucleotide sequence as described herein. In some aspects, the capsid comprises an amino acid sequence having at least 50, 55, 60, 65, 70, 75, 80, 85, 90, 91, 92, 93, 94, 95, 96, 97, 98, 99, or 100% sequence identity to SEQ ID NO: 12, 14, 15 or 16.

In another aspect, the invention provides a viral particle comprising a capsid encoded by a capsid-encoding nucleotide sequence as described herein. In some aspects, the capsid comprises an amino acid sequence having at least 50, 55, 60, 65, 70, 75, 80, 85, 90, 91, 92, 93, 94, 95, 96, 97, 98, 99, or 100% sequence identity to SEQ ID NO: 12, 14, 15 or 16.

### Methods of Ameliorating or Treating a Neuromuscular or Neuromotor Disorder

The AAV capsids identified by the screening methods described herein may be used to develop gene therapies for treating various conditions or disorders.

Accordingly, one aspect of the invention provides a method of ameliorating or treating a neuromuscular or neuromotor condition or disorder in a subject, comprising administering to the subject a therapeutically active amount of an AAV expression vector or viral particle of the invention.

Another aspect of the invention provides a method of ameliorating or treating a neuromuscular or neuromotor disorder in a subject, comprising administering to the subject a therapeutically active amount of an AAV expression vector or viral particle of the invention, wherein the AAV expression vector or viral particle comprises the capsid-encoding nucleotide sequence of an AAV capsid identified by a screening method of the invention, and wherein the iPSCs or ESCs used in the screening method have been derived from the subject. In some embodiments, the iPSCs or ESCs used in the screening method have been derived from a skin sample in the subject.

Consequently, in some embodiments, the AAVs capsids will be “individualised” to the subject, and the method of treatment will be specific (“personalised”) to the subject being treated. One way to test the “individuality” of the AAV capsids identified from the screening methods of the invention is as follows:
(i) Take IPSCs from one strain of mouse (for example, C57/B16) and perform AAV capsid screening in these cells.
(ii) Having identified the genetic sequences of capsids that can infect these C57/B16 motor neurons, test these same capsids for infection of genetically identical mice in vivo (other C57/b16 mice) or genetically similar mice in vivo (for example BALB/c and CD1). Each of these strains of mice will receive muscle injections of the AAV capsid.
(iii) Examine the number of motor neurons infected in each strain. Previous research indicates that AAV capsids evolved in one strain do not show efficient infection in other strains (Horeaux et al., 2018). If this is the case for AAV capsids evolved in the screening methods of the invention, this will indicate that AAV capsids evolved from individual patients will be the most efficient for gene therapies.

In some embodiments, the method involves the AAV expression vector or viral particle retrogradely infecting neurons for the purposes of delivering genetic material to neurons, with the purpose of treating neuromuscular or neuromotor disorders, or disorders affecting movement or targeting neurons for any therapeutic purpose.

In some embodiments, the method comprises altering the activity of neurons in the subject.

In some embodiments, the AAV expression vector or viral particle is delivered intramuscularly, in order to infect motor neurons of a subject neuron retrogradely and alter the activity of the motor neurons in a subject.

As used herein, “retrograde transport” or “retrograde infection” means uptake of the vector at the axon terminal (or “synaptic terminal”), i.e., at the synaptic portion, and transport through the axon in a direction opposite to the direction of propagation of action potentials (and thus “retrograde”) and into the body of the neuron. Subsequently, the viral nucleic acid can enter the nucleus where it can be replicated and become transcriptionally and translationally active.

Such delivery is advantageous when the neuronal cell body and or axon themselves are inaccessible, but their terminal projection fields including synapses, are available for delivery of the genetic vector. Successful delivery to such a terminal projection field of a genetic vector capable of retrograde transport would thus result in retrograde transport and infection of the vulnerable projection neurons.

Once the virus is transported to the body of the neuron, the viral nucleic acid typically localizes to the nucleus of the cell. According to some embodiments of the present invention, adeno-associated viral particles that undergo retrograde transport to the neuronal body can insert their nucleic acid content directly into the nucleus.

Embodiments of the invention involve delivery of a substantially non-toxic, recombinant adeno-associated virus vector having a heterologous gene of interest in order to provide retrograde gene delivery to a neuronal cell body resulting in gene expression.

Many different neurological diseases impair quality of life through their impact on movement. The final common path from the nervous system to muscles can be that of motor neurons, and these neurons are affected in many different disorders, only some of which are classified as “motor neuron diseases.” In some embodiments, AAV expression vectors of the invention are capable of treating a neuromuscular or neuromotor disorder by accessing motor neurons following intramuscular injection, and subsequently modifying gene expression in motor neurons, leading to curing, alleviating symptoms, and/or improving the quality of life of patients with diseases affecting motor neurons.

As used herein, “neurological disorder” refers to a disorder which causes morphological and/or functional abnormality of a neural cell or a population of neural cells. The neurological disorder can result in an impairment or absence of a normal neurological function or presence of an abnormal neurological function in a subject. For example, neurological disorders can be the result of disease, injury, and/or aging. Non-limiting examples of morphological and functional abnormalities include physical deterioration and/or death of neural cells, abnormal growth patterns of neural cells, abnormalities in the physical connection between neural cells, under- or over production of a substance or substances, e.g., a neurotransmitter, by neural cells, failure of neural cells to produce a substance or substances which it normally produces, production of substances, e.g., neurotransmitters, and/or production or transmission of electrical impulses in abnormal patterns or at abnormal times.

As used herein, a “neuromotor disorder” is a developmental or acquired disorder that typically affects movement/gross motor ability, posture, and fine motor ability. The disorder is caused by damage to the central nervous system. This could be due to problems with development or injury to the developing motor pathways in the cortex, basal ganglia, thalamus, cerebellum, brainstem, spinal cord, or peripheral nerve. The most common neuromotor disorders in childhood include cerebral palsy, muscular dystrophy, and spina bifida. The most common neuromotor disorders in adults include stroke, multiple sclerosis, Parkinson's disease and traumatic injury. The impairment may be static (not getting worse) or progressive.

In some embodiments, the neuromuscular or neuromotor disorder is spasticity. As used herein, “spasticity” refers to a condition in which certain muscles are continuously or abnormally contracted. This contraction causes stiffness or tightness of the muscles and can interfere with normal movement of face, limbs, trunk, and/or sphincters, leading to deficits in, for example, speech, gait, and/or bladder and bowel function. Spasticity is a condition that occurs in widespread disorders of the CNS that affect brain and/or spinal cord function, including, for example traumatic injury to brain or spinal cord, multiple sclerosis, cerebral palsy, stroke, or other conditions. Despite the underlying condition, spasticity develops when the properties of motor neurons change in response to the condition, and over-produce electrical impulses, leading to excessive muscle contraction. The damage causes a change in the balance of signals between the nervous system and the muscles, leading to increased excitability in muscles. Spasticity is found in conditions where the brain and/or spinal cord are damaged or fail to develop normally; these include cerebral palsy, multiple sclerosis, spinal cord injury, and acquired brain injury including stroke.

There are several mouse models of spasticity that have been generated, and the ability of the expression vectors of the invention to treat spasticity can be validated using these models. In one embodiment, the ability of the expression vectors to treat spasticity as the result of spinal cord transection (for example see Yoshizaki et al., 2020) can be tested as follows:
(i) Skin biopsies are taken from mice genetically identical to those used for the in vivo spasticity experiments.
(ii) These biopsies are used to generate IPSCs, and in turn motor neurons which are used for the AAV capsid screening methods described herein, to select optimal capsids.
(iii) Mice receive a spinal transection at thoracic levels.
(iv) Spasticity severity is assessed by behavioural observations, such as the Modified Ashworth Scale or by the implantation of EMG recording devices into the muscle.
(v) After transection mice are split into three groups: Group 1 receive an intramuscular injection of an AAV using the capsid derived from the screening process and containing the DNA sequence for a gene intended to reduce motor neuron synaptic firing. Group 2 receive an intramuscular injection of an AAV with the same capsid as group 1, but the DNA will express an inert protein, such as GFP. Group 3 receive an intramuscular injection of an AAV with a wildtype capsid (AAV6) containing the same DNA sequence as Group 1.
(vi) Alterations in spasticity symptoms are monitored before and after AAV intramuscular injection.

This experiment allows for testing whether the in vitro screening process results in AAV capsids that are more efficient at motor neuron infection than those currently available (comparing Groups 1 and 3), and also whether the DNA cargo of the virus is sufficient to reduce spasticity symptoms (comparing Groups 1 and 2).

Another potential animal model is the pig. In this case skin biopsies would be obtained from the animals that are to be used in the experiments and perform the screening and experiments in the same way as with mice.

In certain aspects, the invention also provides the use of expression vectors and viral particles as described herein for the manufacture of a medicament for the treatment of said neuromuscular or neuromotor disorder of a human or animal subject, expression vectors as described herein for use in the treatment of said neuromuscular or neuromotor disorder of a human or animal subject, and methods of treatment of said neuromuscular or neuromotor disorder which comprises administering the expression vectors and viral particles as described herein to an individual in need thereof.

### Administration and Dosage

The viral particles and expression vectors described herein can be delivered to the subject in a variety of ways, such as intramuscularly, intravenously, intracranially, or intraspinally. In some preferred embodiments, said particles and expression vectors described herein can be delivered to the subject via intramuscular injection.

The particular method and site of administration would be at the discretion of the physician who would also select administration techniques using his/her common general knowledge and those techniques known to a skilled practitioner.

Following administration of the viral particles, the recipient individual may exhibit reduction in symptoms of the disease or disorder being treated. For example, for an individual being treated, the recipient individual may exhibit improved neuronal firing, synaptic release of neurotransmitter, survival, growth, or connectivity of the neurons.

The term “treatment,” as used herein in the context of treating a condition, pertains generally to treatment and therapy of a human, in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The viral particle can be delivered in a therapeutically-effective amount.

The term “therapeutically-effective amount” as used herein, pertains to that amount of the viral particle which is effective for producing some desired therapeutic effect, commensurate with a reasonable benefit/risk ratio, when administered in accordance with a desired treatment regimen.

Similarly, the term “prophylactically effective amount,” as used herein pertains to that amount of the viral particle which is effective for producing some desired prophylactic effect, commensurate with a reasonable benefit/risk ratio, when administered in accordance with a desired treatment regimen.

“Prophylaxis” in the context of the present specification should not be understood to describe complete success i.e. complete protection or complete prevention. Rather prophylaxis in the present context refers to a measure which is administered in advance of detection of a symptomatic condition with the aim of preserving health by helping to delay, mitigate or avoid that particular condition.

While it is possible for the viral particle to be used (e.g., administered) alone, it is often preferable to present it as a composition or formulation e.g. with a pharmaceutically acceptable carrier or diluent. In some cases, the viral particle is co-administered with a second expression vector comprising an adeno-viral helper gene, optionally wherein the adeno-viral helper gene is pHelper.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

In some embodiments, the composition is a pharmaceutical composition (e.g., formulation, preparation, medicament) comprising, or consisting essentially of, or consisting of as a sole active ingredient, viral particle as described herein, and a pharmaceutically acceptable carrier, diluent, or excipient.

As described in WO2008096268, in gene therapy embodiments employing delivery of the viral particle, the unit dose may be calculated in terms of the dose of viral particles being administered. Viral doses include a particular number of virus particles or plaque forming units (pfu). For embodiments involving adenovirus, particular unit doses include 10³, 10⁴, 10⁵, 10⁶, 10⁷, 10⁸, 10⁹, 10¹⁰, 10¹¹, 10¹², 10¹³ or 10¹⁴ pfu. Particle doses may be somewhat higher (10 to 100 fold) due to the presence of infection-defective particles.

In some embodiments the methods or treatments of the present invention may be combined with other therapies, whether symptomatic or disease modifying.

The term “treatment” includes combination treatments and therapies, in which two or more treatments or therapies are combined, for example, sequentially or simultaneously.

For example it may be beneficial to combine treatment with a compound as described herein with one or more other (e.g., 1, 2, 3, 4) agents or therapies.

Appropriate examples of co-therapeutics will be known to those skilled in the art on the basis of the disclosure herein. Typically the co-therapeutic may be any known in the art which it is believed may give therapeutic effect in treating the diseases described herein, subject to the diagnosis of the individual being treated.

The agents (i.e. viral particle, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

### Kits

The invention also provides kits that comprise an AAV vector as described herein and one or more viral packaging and envelope expression vectors also described herein. In some embodiments the viral packaging expression vector is an integrase-deficient viral packaging expression vector.

### Cells

The invention also provides a cell comprising an AAV vector as described herein. In some embodiments, this cell is a mammalian cell such as a human cell.

Any sub-titles herein are included for convenience only, and are not to be construed as limiting the disclosure in any way. The invention will now be further described with reference to the following non-limiting Figures and Examples. Other embodiments of the invention will occur to those skilled in the art in the light of these. The disclosure of all references cited herein, inasmuch as it may be used by those skilled in the art to carry out the invention, is hereby specifically incorporated herein by cross-reference. This application claims priority to GB patent application 2010981.5 filed 16 Jul. 2020, contents of which are incorporated herein in their entirety.

### FIGURES

FIG. 1

Therapeutic approach to the treatment of spasticity. Injection of an engineered AAV vector into the muscle infects terminals of motor neurons controlling that muscle (1). The virus is transported to the motor neuron cell body in the spinal cord where a gene is expressed to alleviate symptoms (2).

FIG. 2

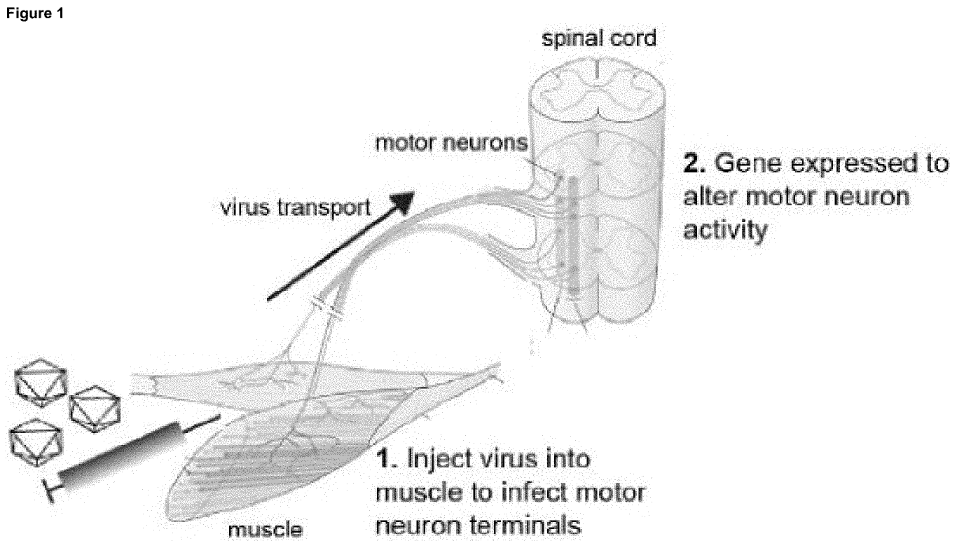

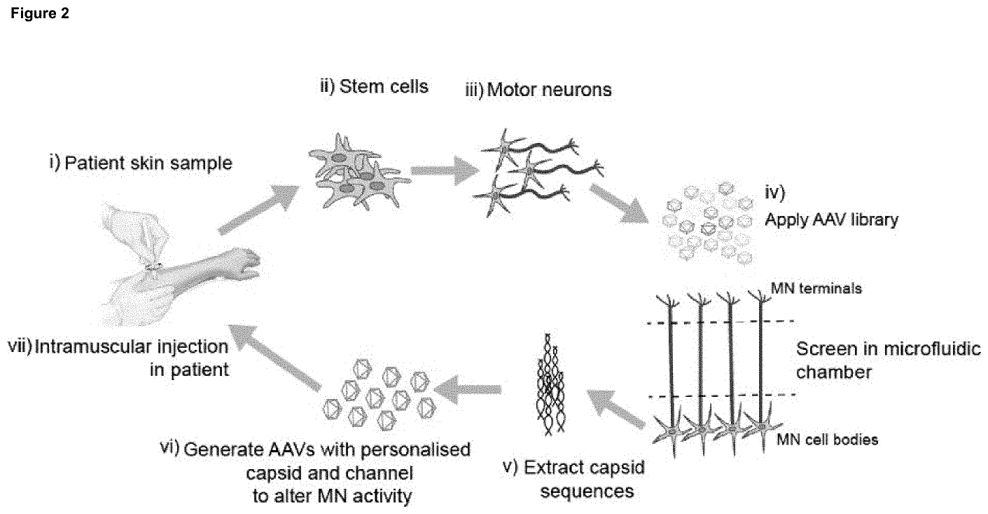

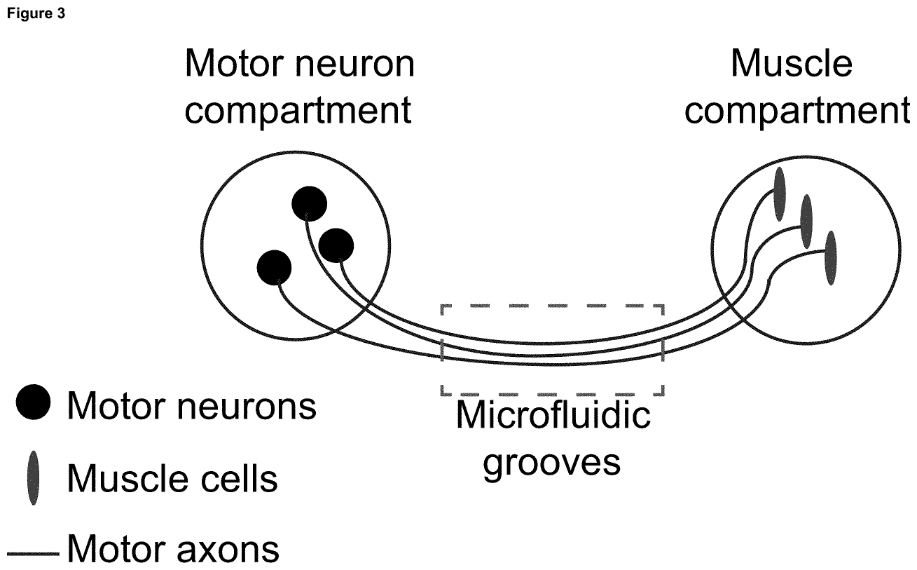

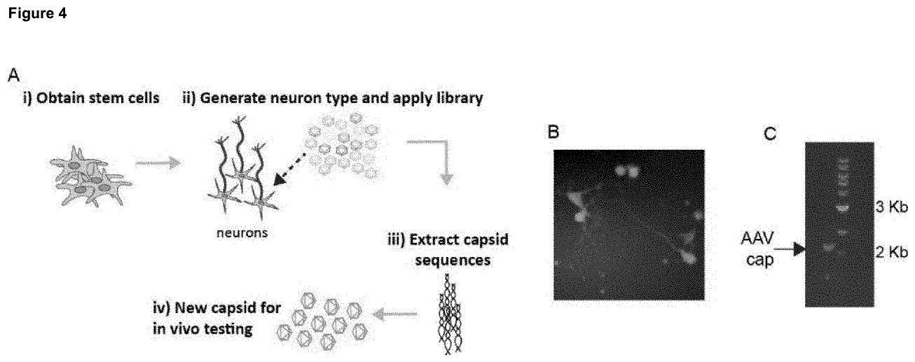

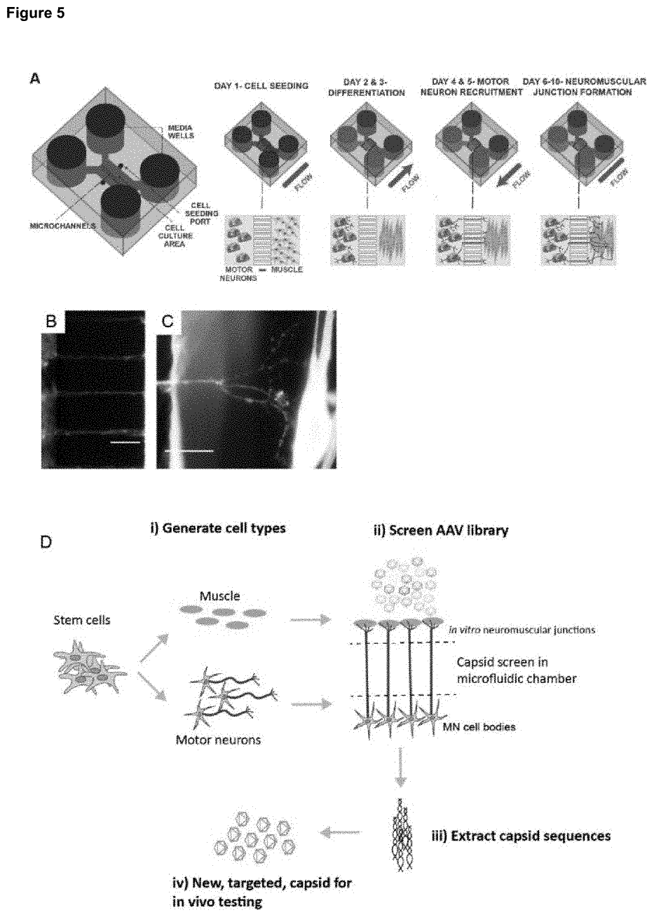

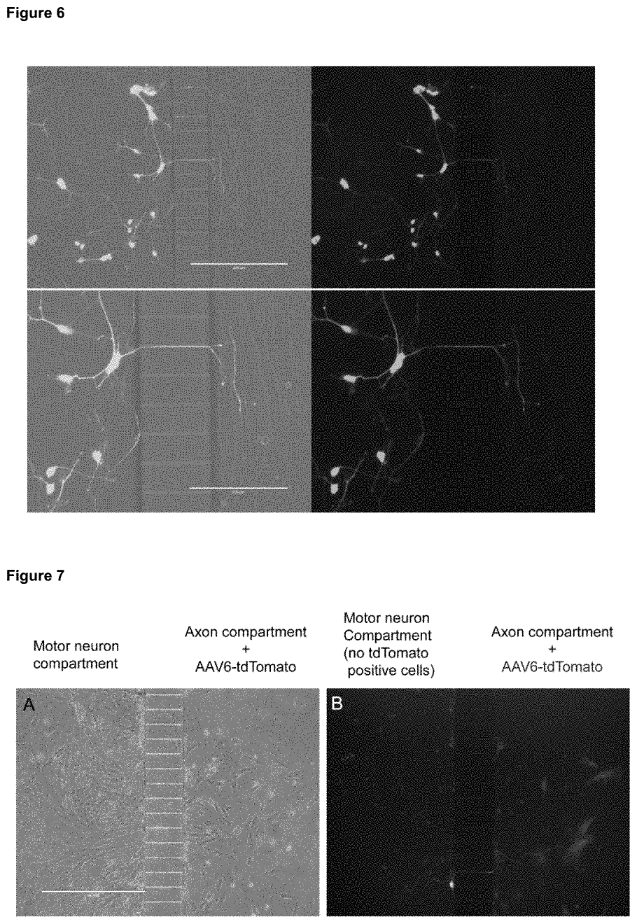

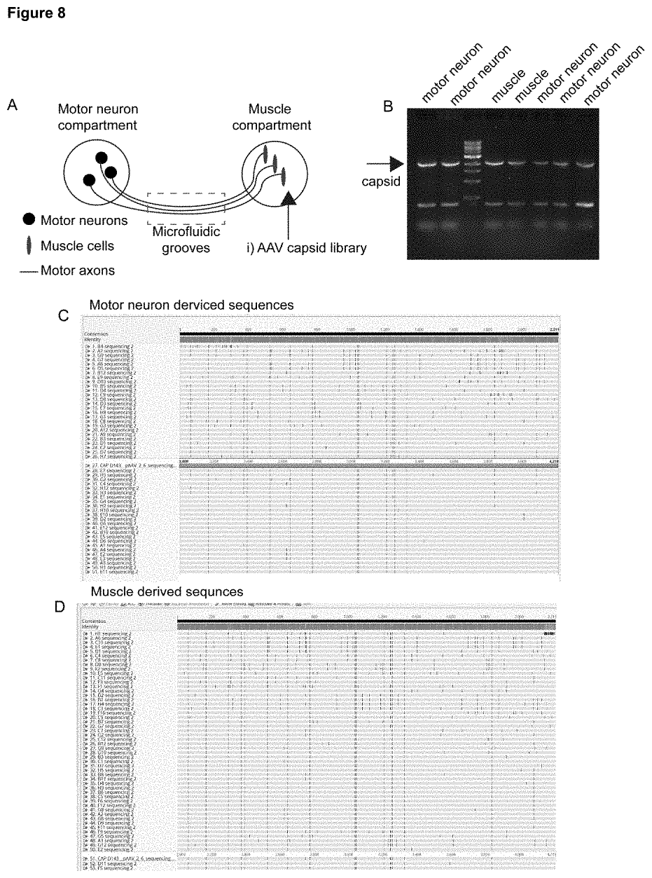

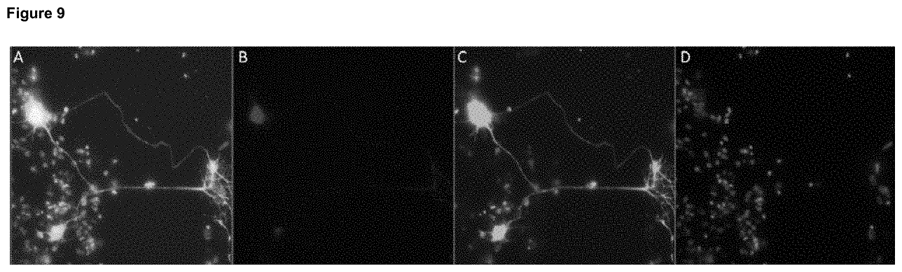

Schematic of example “personalised approach” to AAV capsid screening. Schematic showing potential pipeline for gene therapy to treat spasticity. (i) A skin sample is taken from the patient, (ii) the sample is used to generate iPS cells and, (iii) motor neurons. (iv) An AAV library is screened against the terminals of these motor neurons in vitro and, (v) the efficient retrograde capsid sequences are extracted from motor neuron somata. (vi) The synthetic capsid sequences are used to produce AAV containing a gene to alter motor neuron activity at a GMP facility which is (vii) used for intramuscular injections in the patient.

FIG. 3

Schematic of microfluidic culture of motor neurons and muscle cells.

FIG. 4

(A) General scheme for assaying AAV capsid libraries on stem cell derived neurons. (B) Embryonic stem cell derived motor neurons in culture (expressing GFP under the control of the Hb9 promoter) used for pilot infection with AAV library. (C) DNA agarose gel showing capsid band at 2.2 Kb, DNA was harvested from motor neurons and amplified via PCR

FIG. 5

(A) Example protocol for recreating neuromuscular junction from motor neurons and muscle in vitro. (B) Fluorescent image of neurite processes crossing the central microchannels in microfluidic chambers. (C) Fluorescent image showing a neurite process extending, branching and contacting a muscle fiber. Panels A-C taken from Mills et al., 2018 Molecular Metabolism 7:12-22. D) Example strategy for screening AAV capsid libraries on stem cell derived motor neurons and an in vitro model of the neuromuscular junction.

FIG. 6

Microfluidic device set up.

FIG. 7

Restriction of non-retrograde AAV to the axon compartment.

FIG. 8

Application of AAV capsid library to axonal compartment of the microfluidics chamber and harvesting/bioinformatics of resultant capsid sequences.

FIG. 9

AAVSeqA-tdTomato in motoneurons growing in microfluidic devices.

### EXAMPLES

### Example 1—Identification of Capsid Sequences that Infect Motor Neurons

A general scheme for assaying the AAV capsid libraries on stem cell derived neurons is shown in FIG. 4A.

Using the methods of the disclosure, an AAV library was applied to mouse embryonic stem cell derived motor neurons expressing GFP under the control of the Hb9 promoter (FIG. 4B).

As shown in FIG. 4C, a DNA agarose gel showed the presence of a capsid band at 2.2 kb. This DNA was harvested from the motor neurons.

A combination of PCR and Sanger sequencing to identify the capsid sequences that infected those motor neurons as SEQ ID NO: 1 and SEQ ID NO: 2.

### Prophetic Example 2—Identification of Capsid Sequences that Infect Sub-Compartments of Motor Neurons

Using the methods of the disclosure, this process can be extended and adapted so that AAVs can be identified for their ability to infect sub-compartments of a neuron (for example, the synaptic terminal or axon). Motor neurons, for example, can be grown in microfluidic chambers that separate neuronal axons from cell bodies. In addition, different cell types can be added to this system to more closely recreate in vivo situations, for example the neuromuscular junction (example protocol is shown in FIG. 5A). As described in Mills et al., 2018 Molecular Metabolism 7:12-22, neurites can cross the central microchannels in microfluidic chambers (FIG. 5B and FIG. 5C).

A general strategy for screening AAV capsid libraries on stem cell derived motor neurons in an in vitro model of the neuromuscular junction is shown in FIG. 5D.

The screening method may comprise the following steps:
i) Obtain iPSCs or ESCs (collectively “stem cells”) from an animal, a human subject/patient, or from a cell bank. Derive these stem cells into a specific class of neuron or other cell (for example motor neurons, sensory neurons, dopaminergic neurons, muscle tissue) in culture.
ii) Grow these neurons in a culture system (i.e. microfluidic chambers) that physically separates neuronal cell bodies from other cellular compartments (i.e. neurites, axons).
iii) Further cell types can be added to the culture system, for example sensory neurons and myocytes, so that the system more closely matches the in vivo environment.
iv) Apply an AAV library to one compartment of the culture system (i.e. the compartment containing axons but not cell bodies).
v) Harvest genetic material from a separate compartment, for example the compartment containing neuronal cell bodies, with the purposes of identifying capsid sequences that have successfully infected these cells via (for example) the synaptic terminals.

The obtained genetic sequences could further be used to generate rAAV vectors that use that sequence for capsid proteins. These vectors could be used either for further rounds of directed evolution to increase evolutionary pressure or used for the development of gene therapies.

### Example 3—Co-Culture of Motor Neurons and Muscle Cells in Microfluidic Devices

Motor neurons cultured in microfluidic devices send abundant axons through the microfluidic grooves and contact myotubes (muscle cells). These motor neurons can be maintained in culture for >one week (FIG. 6).

Microdevices were sterilized with ethanol, washed and attached to glass substrates as per the user manual instructions (Xona Microfluidics SND150). Each device was designated a motor neuron (MN) culture compartment and a myotube culture compartment. Both cell culture compartments were initially coated with diluted Matrigel for at least 2 h at 37° C.

C2C12 cells (obtainable from ATCC), an immortalized mouse myoblast cell line, were harvested and seeded first. Myoblasts were resuspended at a density of 12×10⁶ cells per ml and pipetted into the muscle culture compartment (12 ml); resulting in 144,000 myoblasts per device. Myoblast differentiation was initiated after 24 h, by adding myoblast differentiation medium (DMEM containing 5% horse serum). Myoblasts were differentiated for 48 h to form multinucleated fibers. Following myoblast differentiation, motor neurons were harvested, and resuspended at a density of 15×10⁶ cells per ml. Motor neurons were then pipetted into the MN compartment (12 ml); resulting in 180,000 MNs per device. Devices were incubated for 2 h at 37° C. to facilitate cell attachment, followed by addition of their respective medium to fill devices. GDNF and BDNF at 20 ng/ml were added to the muscle compartment, in combination with fluid flow from the muscle to the motor neuron compartment using a 20 μI difference in volume between the compartments, resulting in neurite recruitment.

FIG. 6 shows a fluorescence and bright field merged image of Hb9-mESC derived motor neurons (left side) growing in a matrigel coated compartment and extending axons through the microgrooves, branching and contacting differentiated myotubes (right side).

### Example 4—Restriction of AAV Particles to the Axonal Chamber

Application of non-retrograde AAVs (SEQ ID NO: 13/14) to the muscle compartment results in AAV infection in the muscle compartment only, demonstrating that AAV particles do not cross through the microfluidic groove (FIG. 7).

FIG. 7 shows bright field (FIG. 7A) and fluorescent (FIG. 7B) images of the same motor neuron-microfluiidic culture. AAV6 encoding the red fluorescent protein tdTomato was added to the axonal compartment. Non-neuronal cells in the axonal compartment expressed tdTomato, indicating that they had been infected with the AAV. Cells in the motor neuron compartment did not express tdTomato demonstrating that the AAV itself does not cross the microfluidic barrier.

### Example 5—Harvesting of Capsid Sequences from Motor Neurons and Muscle Cells after Application of AAV Capsid Libraries

Following application of an AAV-capsid library to the axonal side of the microfluidic chamber mutated capsid sequences can be harvested via PCR from both the motor neurons (via retrograde transport) and myotubes (via direct infection).

AAV6 library was applied to the muscle chamber of microfluidic devices (FIG. 8A). Seven days after application neuronal cell bodies were harvested by trypsinisation. The neurons were lysed and used as a PCR template using primers directed against conserved regions of AAV6 capsid. Following PCR of capsid regions (FIG. 8B) the DNA fragment was cloned into a backbone vector and the whole process repeated 3 times to increase evolutionary pressure. The final PCR product of capsid regions were cloned into a DNA vector and submitted for Sanger sequencing. The results were analysed using bioinformatics for highly enriched capsids. Muscle cells were also lysed in the last round and used as a PCR template with the same specific primers with the aim of finding the motoneurons enriched capsids that also infect muscle cells (FIGS. 8C and 8D). Bioinformatic analysis showed several differences in enriched capsids when comparing the motor neuron and muscle infections.

FIG. 8 shows application of AAV capsid library to axonal compartment of the microfluidics chamber and harvesting/bioinformatics of resultant capsid sequences. (FIG. 8A) Experimental design. (FIG. 8B) Representative PCR for AAV capsid on DNA harvested from motor neurons or muscle cells. Capsid is indicated at ˜2 Kb band. (FIG. 8C) Bioinformatic analysis of sequences harvested from motor neurons. Each row is a separate sequence, black lines in columns indicate differences from the parental AAV6 capsid sequence. (FIG. 8D) As (FIG. 8C) except DNA harvested from muscle cells.

### Example 6—In-Vitro Testing of Motor Neuron Enriched Capsid Sequence Co-Culture of Motor Neurons and Muscle Cells in Microfluidic Devices

After bioinformatic analysis, the capsid that appeared with more frequency (8% of the sequences) named Sequence A (SEQ ID NO: 11/12) was selected.

The sequence A capsid was cloned back into the ITR2-REP2 vector and packaged into an AAV expressing td-Tomato fluorescence marker. AAVSeqA-tdTomato was applied to the muscle chamber of microfluidic devices. After 7 days, live imaging (FIG. 9) and fluorescence imaging (FIG. 9) were performed showing the retrograde infection of motor neurons with the new Sequence A capsid AAV virus expressing tdTomato fluorescence marker.

FIG. 9 shows AAVSeqA-tdTomato in motoneurons growing in microfluidic devices. Fluorescence image of Hb9-mESC derived motor neurons infected retrogradely with the AAVSeqA-tdTomato virus. FIG. 9A shows merged image of all the fluorescence channels merged. FIG. 9B shows tdTomato signal, FIG. 9C shows H9-GFP motoneurons and FIG. 9D shows DAPI stained nuclei.

### REFERENCES

Bedbrook C N, Deverman B E, and Gradinaru V. Viral Strategies for Targeting the Central and Peripheral Nervous Systems. Annual Review of Neuroscience 2018 41:1, 323-348
Buning H, Huber A, Zhang L, Meumann N, Hacker U Engineering the AAV capsid to optimize vector-host-interactions. Current Opinion in Pharmacology, 24, (2015), 94-104, https://doi.org/10.1016/j.coph 0.2015.08.002.
Dalkara et al., In Vivo-Directed Evolution of a New Adeno-Associated Virus for Therapeutic Outer Retinal Gene Delivery from the Vitreous. Science Translational Medicine 2013:Vol. 5, Issue 189, pp. 189ra76 DOI: 10.1126/scitranslmed.3005708
Davidsson et al., A systematic capsid evolution approach performed in vivo for the design of AAV vectors with tailored properties and tropism. Proceedings of the National Academy of Sciences Dec 2019, 116 (52) 27053-27062; DOI: 10.1073/pnas.1910061116
Deverman et al., Cre-dependent selection yields AAV variants for widespread gene transfer to the adult brain. Nature Biotechnology. 2016 34(2):204-9. doi: 10.1038/nbt.3440.
Hordeaux et al., The Neurotropic Properties of AAV-PHP.B Are Limited to C57BL/6J Mice. Molecular Therapy. 26(3):664-668. doi: 10.1016/j.ymthe.2018.01.018.
Li, C., Samulski, R. J. Engineering adeno-associated virus vectors for gene therapy. Nature Reviews Genetics 21, 255-272 (2020). https://doi.org/10.1038/s41576-019-0205-4
Pulicherla et al., Engineering liver-detargeted AAV9 vectors for cardiac and musculoskeletal gene transfer. Molecular Therapy. 2011 19(6):1070-8. doi: 10.1038/mt.2011.22.
Tervo et al., A Designer AAV Variant Permits Efficient Retrograde Access to Projection Neurons. Neuron. 92(2):372-382. doi: 10.1016/j.neuron.2016.09.021.
Vandenberghe, L., Wilson, J. & Gao, G. Tailoring the AAV vector capsid for gene therapy. Gene Ther 16, 311-319 (2009). https://doi.org/10.1038/gt.2008.170
Yang et al., A myocardium tropic adeno-associated virus (AAV) evolved by DNA shuffling and in vivo selection. Proceedings of the National Academy of Sciences March 2009, 106 (10) 3946-3951; DOI: 10.1073/pnas.0813207106

Sequence Annex

Nucleotide sequence of capsid sequence harvested from ES/iPSC-derived motor
neurons (SEQ ID NO: 1)
ATGGCTGCCGATGGTTATCTTCCAGATTGGCTCGAGGACAACCTCTCTGAGGGCATTCGCGAGTGGTGGGAC
TTGAAACCTGGAGCCCCGAAACCCAAAGCCAACGAGCAAAAGCAGGACGACGGCCGGGGTCTGGTGCTTCCT
GGCTACAAGTACCTCGGACCCTTCAACGGACTCGACAAGGGGGAGCCCGTCAACGCGGCGGATGCAGCGGC
CCTCGAGCACGACAAGGCCTACGACCAGCAGCTCAAAGCGGGTGACAATCCGTACCTGCGGTATAACCACGC
CGACGCCGAGTTTCAGGAGCGTCTGCAAGAAGATACGTCTTTTGGGGGCAACCTCGGGCGAGCAGTCTTCCA
GGCCAAGAAGAGGGTTCTCGAACCTCTTGGTCTGGTTGAGGAAGGTGCTAAGACGGCTCCTGGAAAGAAACG
TCCGGTAGAGCAGTCGCCACAAGAGCCAGACTCCTCCTCGGGCATTGGCAAGACAGGCCAGCAGCCCGCTA
AAAAGAGACTCAATTTTGGTCAGACTGGCGACTCAGAGTCAGTCCCCGACCCACAACCTCTCGGAGAACCTCC
AGCAACCCCCGCTGCTGTGGGACCTACTACAATGGCTTCAGGCGGTGGCGCACCAATGGCAGACAATAACGA
AGGCGCCGACGGAGTGGGTAATGCCTCAGGAAATTGGCATTGCGATTCCACATGGCTGGGCGACAGAGTCA
TCACCACCAGCACCCGAACATGGGCCTTGCCCACCTATAACAACCACCTCTACAAGCAAATCTCCAGTGCTTCA
ACGGGGGCCAGCAACGACAACCACTACTTCGGCTACAGCACCCCCTGGGGGTATTTTGATTTCAACAGATTCC
ACTGCCATTTCTCACCACGTGACTGGCAGCGACTCATCAACAACAATTGGGGATTCCGGCCCAAGAGACTCAA
CTTCAAGCTCTTCAACATCCAAGTCAAGGAGGTCACGACGAATGATGGCGTCACGACCATCGCTAATAACCTT
ACCAGCACGGTTCAAGTCTTCTCGGACTCGGAGTACCAGTTGCCGTACGTCCTCGGCTCTGCGCACCAGGGT
TGCCTCCCTCCGTCCCCGGCGGACGTGTTCATGATTCCTCAGTACGGCTACCTAACGCTCAACAATGGCAGCC
AGGCAGTGGGACGGTCATCCTTTTACTGCCTGGAATATTTCCCATCGCAGATGCTGAGAACGGGCAATAACTT
TACCTTCAGCTACACCTTCGAGGACGTGCCTTTCCACAGCAGCTACGCGCACAGCCAGAGCCTGGACCGGCT
GATGAATCCTCTCATCGACCAGTACCTGTATTACCTGAACAGAACTCAGAATCAGTCCGGAAGTGCCCAATACA
AGGACTTGCTGTTTAGCCGGGGGTCTCCAGCTGGCATGTCTGTTCAGCCCAAAAACTGGCTACCTGGACCCTG
TTACCGGCAGCAGCGCGTTTCTAAAACAAAAACAGACAACAACAACAGCAACTTTACGTGGACTGGTGCTTCAA
AATATAACCTTAATGGGCGTGAATCTATAATCAACCCTGGCACTGCTATGGCCTCACACAAAGACGACAAAGAC
AAGTTCTTTCCCATGAGCGGTGTCATGATTTTTGGAAAGGAGAGCGCGGGAGCTTCAAACACTGCATTGGACA
ATGTCATGATCACAGACGAAGAGGAAATCAAAGCCACTAACCCCGTGGCCACCGAAAGATTTGGGACTGTGGC
AGTCAATCTCCAGAGCAGCAGCACAGACCCTGCGACCGGAGATGTGCATGTTATGGGAGCCTTACCTGGAATG
GTGTGGCAAGACAGAGACGTATACCTGCAGGGTCCTATTTGGGCCAAAATTCCTCACACGGATGGACACTTTC
ACCCGTCTCCTCTCATGGGCGGCTTTGGACTTAAGCACCCGCCTCCTCAGATCCTCATCAAAAACACGCCTGT
TCCTGCGAATCCTCCGGCAGAGTTTTCGGCTACAAAGTTTGCATCATTCATCACCCAGTATTCCACAGGACAAG
TGAGCGTGGAGATTGAATGGGAGCTGCAGAAAGAAAACAGCAAACGCTGGAATCCCGAAGTGCAGTATACAT
CTATCTATGCAAAATCTGCCAACGTTGATTTCACTGTGGACAACAATGGACTTTATACTGAGCCTCGCCCCAT
TGGCACCCGTTACCTCACCCGTCCCCTGTAA
Nucleotide sequence of capsid sequence harvested from ES/iPSC-derived motor
neurons (SEQ ID NO: 2)
ATGGCTGCCGATGGTTATCTTCCAGATTGGCTCGAGGACAACCTCTCTGAGGGCATTCGCGAGTGGTGGGAC
TTGAAACCTGGAGCCCCGAAACGCAAAGCCAACCAGCAAAAGCAGGACGACGGCCGGGGTCTGGTGCTTCCT
GGCTACAAGTACCTCGGACCCTTCAACGGACTCGACAAGGGGGAGCCCGTCAACGCGGCGGATGCAGCG
GCCCTCGAGCACGACAAGGCCTACGACCAGCAGCTCAAAGCGGGTGACAATCCGTACCTGCGGTATAACCAC
GCCGACGCCGAGTTTCAGGAGCGTCTGCAAGAAGATACGTCTTTTGGGGGCAACCTCGGGCGAGCAGTCTTC
CAGGCCAAGAAGAGGGTTCTCGAACCTCTTGGTCTGGTTGAGGAAGGTGCTAAGACGGCTCCTGGAAAGAAA
CGTCCGGTAGAGCAGTCGCCACAAGAGCCAGACTCCTCCTCGGGCATTGGCAAGACAGGCCAGCAGCCCGC
TAAAAAGAGACTCAATTTTGGTCAGACTGGCGACTCAGAGTCAGTCCCCGACCCACAACCTCTCGGAGAACCT
CCAGCAACCCCCGCTGCTGTGGGACCTACTACAATGGCTTCAGGCGGTGGCGCACCAATGGCAGACAATAAC
GAAGGCGCCGACGGAGTGGGTAATGCCTCAGGAAATTGGCATTGCGATTCCACATGGATGGGCGACAGAGT
CATCACCACCAGCACCCGAACATGGGCCTTGCCCACCTATAACAACCACCTCTACAAGCAAATCTCCAGTGCTT
CAACGGGGGCCAGCAACGACAACCACTACTTCGGCTACAGCACCCCCTGGGGGTATTTTGATTTCAACAGATT
CCACTGCCATTTCTCACCACGTGACTGGCAGCGACTCATCAACAACAATTGGGGATTCCGGCCCAAGAGACTC
AACTTCAAGCTCTTCAACATCCAAGTCAAGGAGGTCACGACGAATGATGGCGTCACGACCATCGCTAATAACC
TTACCAGCACGGTTCAAGTCTTCTCGGACTCGGAGTACCAGTTGCCGTACGTCCTCGGCTCTGCGCACCAGGG
CTGCCTCCCTCCGTTCCCGGCGGACGTGTTCATGATTCCGCAGTACGGCTACCAAACGCTCAACAATGGCAGC
CAGGCAGTGGGACGGTCATCCTTTTACTGCCTGGAATATTTCCCATCGCAGATGCTGAGAACGGGCAATAACT
TTACCATCAGCTACACCTTCGAGGACGTGCCTTTCCACAGCAGCTACGCGCACAGCCAGAGCCTGGACCGGC
TGATGAATCCTCTCATCGACCAGTACCTGTATTACCTGAACAGAACTCAGAATCAGTCCGGAAGTGCCCAAAAC
AAGGACTTGCTGTTTAGCCGGGGGTCTCCAGCTGGCATGTCTGTTCAGCCCAAAAACTGGCTACCTGGACCG
TGTTACCGGCAGCAGCGCGTTTCTAAAACATAAACAGACAACAACAACAGCAACTTTACCTGGACTGGTGCTTC
AAAATATAACCTTAATGGGCGTGAATCTATAATCAACCCTGGCACTGCTATGGCCTCACACAAAGACGACAAAG
ACAAGTTCTTTCCCATGAGCGGTGTCATGATTTTTGGAAAGGAGAGCGCCGGAGCTTCAAACACTGCATTGGA
CAATGTCATGATCACAGACGAAGAGGAAATCAAAGCCACTAACCCCGTGGCCACCGAAAGATTTGGGACTGTG
GCAGTCAATCTCCAGAGCAGCAGCACAGACCCTGCGACCGGAGATGTGCATGTTATGGGAGCCTTACCTGGAA
TGGCGTGGCAAGACAGAGACGTATACCTGCAGGGTCCTATTTGGGCCAAAATTCCTCACACGGATGGACACTT
TCACCCGTCTCCTCTCATGGGCGGCTTTGGACTTAAGCACCCGCCTCCTCAGATCCTCATCAAAAACACGCCT
GTTCCTGCGAATCCTCCGGCAGAGTTTTCGGCTACAAAGTTTGCTTCATTCATCACCCAGTATTCCACAGGACA
AGTGAGCGTGGAGATTGAATGGGAGCTGCAGAAAGAAAACAGCAAACGCTGGAATCCCGAAGTGCAGTATAC
ATCTAACTATGCAAAATCTGCCAACGTTGATTTCACTGTGGACAACAATGGACTTTATACTGAGCCTCGCCCC
ATTGGCACCCGTTACCTCACCCGTCCCCTGTAA
Nucleotide sequence of KCC2 gene (SEQ ID NO: 3)
(GenBank: AF208159.1)
ATGCCCAACAACCTGACGGACTGCGAGGACGGCGATGGGGGAGCCAACCCGGGTGATGGCAACCCCAAG
GAAAGCAGTCCCTTCATCAACAGCACCGACACAGAGAAGGGAAAGGAGTATGATGGCAAGAACATGGCCTT
GTTTGAGGAGGAGATGGACACCAGCCCTATGGTGTCCTCCTTGCTCAGTGGCCTGGCCAACTACACCAACC
TGCCCCAGGGAAGTAGGGAGCATGAAGAGGCAGAAAACAATGAGGGTGGAAAAAAGAAGCCGGTGCAGGC
CCCACGCATGGGCACCTTCATGGGCGTGTACCTGCCGTGCCTGCAGAACATCTTTGGCGTCATCCTCTTCC
TGCGGCTCACCTGGGTGGTGGGCATTGCAGGCATCATGGAGTCCTTCTGCATGGTGTTCATCTGCTGCTCC
TGTACGATGCTCACGGCCATCTCCATGAGTGCAATTGCAACGAATGGTGTTGTGCCTGCTGGTGGCTCCTA
CTACATGATTTCCAGGTCTCTGGGCCCAGAGTTTGGGGGTGCCGTGGGCCTCTGCTTCTACCTGGGCACTA
CCTTTGCAGGAGCCATGTACATCCTGGGCACCATCGAAATCCTGCTGGCTTACCTCTTCCCAGCCATGGCC
ATCTTCAAGGCAGAAGATGCCAGTGGGGAGGCAGCAGCCATGCTGAACAACATGCGTGTTTACGGCACCTG
TGTGCTCACCTGCATGGCCACTGTGGTGTTTGTGGGTGTCAAGTATGTCAACAAGTTTGCCCTTGTCTTCCT
GGGTTGTGTCATCCTCTCCATCCTGGCCATCTATGCTGGGGTCATCAAGTCTGCCTTCGACCCACCCAACTT
CCCGATCTGCCTCCTGGGTAACCGCACGCTGTCTCGCCATGGCTTTGATGTCTGTGCCAAGCTGGCTTGGG
AAGGAAATGAGACGGTGACCACACGGCTATGGGGCCTTTTCTGCTCCTCTCGCTTCCTCAACGCCACCTGT
GATGAATACTTCACCCGAAACAATGTCACAGAGATCCAGGGCATCCCTGGTGCTGCCAGTGGCCTCATCAA
AGAGAACCTCTGGAGCTCCTACCTGACCAAGGGCGTGATTGTGGAGAGGAGTGGGATGACCTCGGTGGGC
CTGGCCGATGGCACTCCTATCGACATGGACCACCCTTATGTCTTCAGTGATATGACCTCCTACTTCACCCTG
CTGGTTGGCATCTACTTCCCCTCAGTCACAGGGATCATGGCTGGTTCTAACCGCTCTGGGGACCTGAGGGA
TGCCCAGAAGTCAATCCCCACTGGCACCATCCTGGCCATCGCCACCACCTCTGCTGTCTACATCAGCTCCG
TTGTTCTGTTTGGGGCCTGCATTGAGGGGGTCGTCCTGCGGGACAAGTTTGGCGAAGCTGTGAATGGCAAC
CTCGTGGTGGGCACTCTGGCCTGGCCATCTCCATGGGTAATTGTCATCGGATCCTTCTTCTCCACCTGTGG
GCTGGGCTGCAGAGCCTCACGGGGGCCCCACGCCTGCTGCAGGCCATCTCGAGGGATGGCATTGTGCCCT
TCCTGCAGGTCTTTGGCCATGGCAAGGCCAATGGAGAGCCGACCTGGGCCCTGCTCCTGACTGCCTGCAT
CTGCGAGATTGGCATCCTCATTGCATCCCTCGACGAGGTGGCCCCCATCCTCTCTATGTTCTTCCTGATGTG
CTACATGTTTGTGAATCTGGCCTGTGCAGTGCAGACGCTGCTGAGGACACCCAACTGGAGGCCACGCTTTC
GATATTACCACTGGACCCTCTCCTTCCTGGGCATGAGCCTCTGCCTGGCCCTCATGTTCATCTGCTCCTGGT
ATTATGCACTGGTAGCCATGCTCATTGCTGGACTCATCTACAAGTACATTGAGTACCGTGGGGCAGAGAAGG
AGTGGGGCGATGGGATACGAGGTCTGTCTCTCAGTGCGGCTCGCTATGCCCTCTTACGCCTGGAGGAAGG
GCCCCCACACACCAAGAACTGGAGGCCACAGCTGCTGGTGCTGGTGCGTGTGGACCAAGACCAGAATGTG
GTGCACCCCCAGCTGCTCTCACTGACCTCCCAGCTGAAGGCAGGGAAGGGCCTGACCATCGTGGGCTCTG
TCCTTGAGGGCACCTTTCTGGAAAATCATCCACAGGCCCAGCGGGCAGAAGAGTCTATCAGGCGCCTGATG
GAGGCAGAGAAGGTGAAGGGCTTCTGCCAGGTGGTGATCTCCTCCAACTTGCGTGATGGCGTGTCCCATCT
GATCCAGTCCGGGGGCCTCGGGGGGCTGCAGCACAACACTGTGCTTGTTGGCTGGCCCCGCAACTGGCG
CCAGAAGGAAGATCATCAGACGTGGAGGAACTTCATTGAGCTGGTCCGGGAAACCACAGCTGGCCACTTAG
CCCTGCTGGTCACCAAGAACGTTTCCATGTTTCCTGGGAACCCTGAGCGCTTCTCTGAGGGCAGCATCGAC
GTTTGGTGGATTGTGCACGATGGAGGCATGCTCATGCTGCTGCCCTTCCTGCTGCGGCACCACAAGGTCTG
GCGGAAGTGCAAGATGCGTATCTTCACTGTGGCCCAGATGGATGACAATAGCATCCAGATGAAGAAGGATC
TGACCACATTTCTGTATCATTTACGCATCACTGCGGAGGTCGAGGTGGTGGAGATGCATGAGAGCGACATCT
CAGCTTACACCTATGAGAAGACGTTGGTGATGGAGCAGCGTTCCCAGATCCTCAAACAGATGCATTTAACCA
AGAATGAGCGGGAGCGGGAGATCCAGAGTATCACAGATGAGTCACGAGGCTCAATCCGGAGAAAGAATCC
AGCCAACACGCGGCTCCGCCTGAACGTCCCAGAAGAGACGGCTGGTGACAGTGAAGAGAAGCCAGAGGAG
GAGGTGCAGCTGATCCACGATCAGAGTGCTCCCAGCTGCCCCAGCAGCTCCCCGTCCCCAGGGGAGGAGC
CTGAGGGGGAAGGGGAGACAGATCCGGAGAAGGTGCATCTCACCTGGACCAAGGACAAGTCGGTGGCAGA
GAAGAATAAGGGCCCCAGTCCTGTCTCCTCTGAGGGCATCAAGGACTTCTTCAGCATGAAGCCGGAGTGGG
AGAACTTGAACCAGTCCAACGTGCGGCGCATGCACACGGCCGTGCGGCTGAACGAGGTCATCGTGAAGAA
ATCCCGGGACGCCAAGCTTGTTTTGCTCAACATGCCTGGGCCTCCCCGCAACCGCAATGGTGATGAAAACT
ACATGGAGTTTCTCGAGGTCCTCACAGAGCACCTGGACCGGGTGATGCTGGTCCGCGGTGGTGGCCGAGA
GGTCATCACCATCTACTCCTGAGAACCAGGTCCTGCCACCCGGGCCCGAGCGCGCCCGGCCCGCGGCTCC
GGAGCCCTCGCCGCGCCCCCCGCCGCTGTCACCGTTTACATACAGACCCTGTGCCCGTGTCCTGGCCCCT
TACCCCGCTGCCTGAAGCCCGGAGGCCACGCCTGTTGGGGCTGATTCGGAGAGGGCGCCCCGCCGCGCA
GAGACCAGAGCTCCTCAGTGCCAGTTTGGCCCCTGGGTCTTCGCTGCCCTTTTTCTAAGCCCGGCCTCGTC
TCGCCGGAGGAGACGCTGCAATAAAGGTTGGGAGAAGGCGCGGAAAGGAGAGGAGCTGGGGCCTTGGGG
ACCCCCAGGTAGTCCATGCGGCCCATTCCTCCCCTTCCCACTCCCGCCGCGGTCCTCGCTCTGCGCTCCTC
CGGCGCTGCTCCCTGGCTCCCGGCGGCCCGGAGGCCCGCGGGGTGGGAAGGCCGCGCTTGCCGTCTCC
GCCGCCCCTTCTCGCCGAGCCGTGGGGCGCGGGCGGCCGAGCCTATACATAGTGTACAGGAGACATCGC
GTGTATTTTTAACGTCCCCATATTTATGTGACTAGAAGCGCAACAGACTTCTCGCCATAGTCGAGCTCTCCCG
CTGGGGGCACTGCGGGGAGGCGAGGCCTCGGGAAGCTGAATTTTCCTTGACGTCCAAGAGTTTGAGAGCG
AAAGTGCTTTAGGCCCAGGCGGGGGTCGTGGCCTCGTTCCCTCGACACCTCCGTCCTGCTCTCGCCTCTTC
GCCCTTTCCGCGCGCCCTTGGCTTCCCACCCTCCTCTCCAGTCCTTTTCCGAGATGAGGTGAGACAAGGGT
CCAACTTTTCCTGGATTCGCCTCCCAGCGGACGTGAGCTTCCACTGCGGCTGCAGAGACGCGAGCAACCTC
TTCTCATCGGCTCTTATGCAAGTTGGGGCCAGGATAGGGGAGGGGTGCTCCTCAAGAGGAAGAAACCGAGA
GGCCCGCGCCCCACCGAGGAAGCCCCGCCCCGGTGCCTTCGCTGGGGAGCAGGCGTCTCTCCTCAGTCG
GCTTGTCGCCTGCTCCCCGTATCCCATGGCTCCTCGCCAAAGACTGAAATTGTGGAGCTGGAGGGCGCCCC
CTCCCCGGAGTTTCCTCCCTGGGACAAGTGAGGGAGGAGGGGGCCGATTCTGGTTTAGGGGCCGGACCCA
CTGAGAGGCCCCAGAGCCGCCCGTGATGTTCCTCCCCCGTCCCCATCTGGCAGCTCCTGTCTCGCCTGAG
GGACCCAGCCGCCTTCTCCGTGCTCTGGGGCCGGGCCTCGCTGCTTAGCAGCGGCCTCTAGCTCCGTCTC
CCGGGGACCTGGGCCTGAGGGAGGGCTGGAGTCAGCACGCGCTTTGTCCTTAGCGCCTGTCTGCTCTCCT
CTAACTAGGACCCAGGGCCTTTGGCTTCCCCAGCTCATCCTTGGCCCTTCCGCTCCACCAGCCTGGTCTGA
GGCGTGCTCTGTCCTTAGAGAAGGCGCGGTGGCCGGGTTCCCTTCCCCTAGGGCACATTACTAAGGGGGT
CAGGCACTGCATGCTCGTTCCAGCACCATCTGGGACTGGGTACAGTACCTCCAGCCCCAGGGCCCTGACCT
GCGCACCTAGCTTGACATCTCACGCACCTCCCAGAGCTGGCGCCACTGAGTAATCCGGACCTCACCACCTC
TTTTCCTTTGAGCCCAAGGCAGAGCTAGAGCTGGAGCTGGCGCCACCCAGACAGCGTCAGGTGTGGCTGG
GGTAGGTTTGGAGGTCTGCCAGTTACGCCAAGTCCCCTCTGAGATTCGATCAGGGGACTGGATAGATTCTTT
CAGGTACTCAATCAGGAAGCTGGAGGTGTTAGACACCAGCCCCCTGCATCCTTCAGTAGACCTCCCTCTGA
ACACCACAGCCAGGTCCTGCCTTCTGGGGGCCTGAATATTCCAGAGCTGATGTGATGGGCTGTGCAGAAGG
GGGCTGTATCAACATCAATTAGGGAACCAAAGTTGCACTATCTGGGCCCAGATTGTCTGGTTGGCAAGAGCA
AAGTTTCCGTTGATGAAACAGACATCCCACAACAAAAACCCAAGTTTTCTGTGCTACATGTGCAATATTTGTT
ATGAATGTTATCACAAGTCATTCATCAAGTTATCTTTATAATCACTGTAGTTAGATGTTTCATGTCCATTCAAGT
GACTTTTATTCTGAGTGCAATATTTCAATAGCCTTGTAGTGATAACTAGTGTTGCTTTTGTTTAGATGATCTAT
GTGCAGGGCAATGCAATGAAGTTGAAACCCCTTGGTAATAGGAGAGGTTGCAAACCAAATCAAGAGTATTTA
TTACTATTACTGCTATTATTATTAGGCCTGCCTTTAATTTTCAGTGTAAGTGTTCAGTATGCCGCATCCTGCCT
CAGTATTGATCTTGTGTTCTTTGTGCCAATATGAAAAGGAGAGGGTTGGTTCTTTCCTTTATTGTTGAATGCT
CCCATTTAATGCTTTATGGCTTTTACTGTATTACTTTTTTAGACTCCCGTCTGCACAAAATGCAATAAAAATAAT
TTTATTATAAAAAAAAAAA
Amino acid sequence of KCC2 gene product (SEQ ID NO: 4)
MPNNLTDCEDGDGGANPGDGNPKESSPFINSTDTEKGKEYDGKNMALFEEEMDTSPMVSSLLSGLANYTNLPQ
GSREHEEAENNEGGKKKPVQAPRMGTFMGVYLPCLQNIFGVILFLRLTWVVGIAGIMESFCMVFICCSCTMLTAIS
MSAIATNGVVPAGGSYYMISRSLGPEFGGAVGLCFYLGTTFAGAMYILGTIEILLAYLFPAMAIFKAEDASGEAAAM
LNNMRVYGTCVLTCMATVVFVGVKYVNKFALVFLGCVILSILAIYAGVIKSAFDPPNFPICLLGNRTLSRHGFDVCA
KLAWEGNETVTTRLWGLFCSSRFLNATCDEYFTRNNVTEIQGIPGAASGLIKENLWSSYLTKGVIVERSGMTSVG
LADGTPIDMDHPYVFSDMTSYFTLLVGIYFPSVTGIMAGSNRSGDLRDAQKSIPTGTILAIATTSAVYISSVVLFGAC
IEGVVLRDKFGEAVNGNLVVGTLAWPSPWVIVIGSFFSTCGAGLQSLTGAPRLLQAISRDGIVPFLQVFGHGKAN
GEPTWALLLTACICEIGILIASLDEVAPILSMFFLMCYMFVNLACAVQTLLRTPNWRPRFRYYHWTLSFLGMSLCLA
LMFICSWYYALVAMLIAGLIYKYIEYRGAEKEWGDGIRGLSLSAARYALLRLEEGPPHTKNWRPQLLVLVRVDQDQ
NVVHPQLLSLTSQLKAGKGLTIVGSVLEGTFLENHPQAQRAEESIRRLMEAEKVKGFCQVVISSNLRDGVSHLIQS
GGLGGLQHNTVLVGWPRNWRQKEDHQTWRNFIELVRETTAGHLALLVTKNVSMFPGNPERFSEGSIDVWWIVH
DGGMLMLLPFLLRHHKVWRKCKMRIFTVAQMDDNSIQMKKDLTTFLYHLRITAEVEVVEMHESDISAYTYEKTLV
MEQRSQILKQMHLTKNEREREIQSITDESRGSIRRKNPANTRLRLNVPEETAGDSEEKPEEEVQLIHDQSAPSCP
SSSPSPGEEPEGEGETDPEKVHLTWTKDKSVAEKNKGPSPVSSEGIKDFFSMKPEWENLNQSNVRRMHTAVRL
NEVIVKKSRDAKLVLLNMPGPPRNRNGDENYMEFLEVLTEHLDRVMLVRGGGREVITIYS
Nucleotide sequence of Kv1 gene (SEQ ID NO: 5)
(NM_000217 XM_001133050)
AGAAATGGACCGAGCGGACCCGCCGCCGCACGCACCCTGCTCCACTCCAAGCTCCTAAGGGCTCCTGGCG
CGCCGCGTAGCCTTGGCGAGGTCCGCGCTGGGGTGCGGAGAGCGAAGGGAACTGGAGAGCCATGTAGAT
CCAGGCTCTCGCCCGCCCGCCTCCTTCGGGATCGAATCAAGGGCTCCCATAGTGTTAGGAGGGGGCGAGA
GTGCTGTTTATCGTCATTTGCCTCGGAGCTTCGAGAGAGGGTGGTATTTTGCTTTTCCGCCCCGCATCCTCC
GGAACTCCCTGCACCGGAGAGAGGACGGCGTCTCCAGGTTGCTGGCAACCGGTGAGAATGGGGGTAGGG
AAGGAACATTTTCGCCGTAGCTGCTCCGTAAAGCGATTGTCCAACTGAGAGGGGCGTCGGACGAGTGGACC
AGGGCGGCGAGTTTGCCCGGCGCGTCTCGGATGCTGCTGCGGCGGCCGCCGCGGCTCCCGCCAGGGCA
CTGCAAAGACGACCTGCCGCATTCCCACTCGGGCTCTCCGCTGACTCAGCACCGCCCCTGCGCCAAGCCA
GCCGGCCAGCGTGCATCGATCGCCCTGGTGGGAGCTTAGAAGGCGGCAGGCGAAGAGGGGTAGGAGGGG
GGAGAGCCGAGGAGAAGCAGAGAGGGTGGCAGGCGTGGGGATCTGCCGAGCCGGCACTGCACCGGGTC
CTAGGAAGGCTCTCGGAGGGGAGGGGAGGCCAGGGCGACCCCCGAAGCAATGGCCCAGTCCGCTAGAAC
GGCACTGCGTTAAGGCACCTGGGATCAGGAAGAAATATCTAAACAACAACAACAGAAAACCAACAAACCCCC
AAACCCAAACCCAACCCTCTGCAAAAAGCTGCACCCGGCCCGCAGGCGAGGGGGATTCCAAACTGAGTGA
AAGGCAGGGTGGAGGGGAAGGCAGCGAGAGGCAAAGTCGCAGATCTCCCGACCTGCTCGTGTTGAAGCAC
CTCCCCCTGGGCGTGAGGGAGACGCGCGCTCCGGTGGGGGGGCCGCTTGGGTCCCCCCCACCCCTGGTC
CCTGGCTGCTTCCCACCCCGGGCTCTCTCCTGGCCTCCCACCCCCGCGCCCGGCTTCCACCATGACGGTG
ATGTCTGGGGAGAACGTGGACGAGGCTTCGGCCGCCCCGGGCCACCCCCAGGATGGCAGCTACCCCCGG
CAGGCCGACCACGACGACCACGAGTGCTGCGAGCGCGTGGTGATCAACATCTCCGGGCTGCGCTTCGAGA
CGCAGCTCAAGACCCTGGCGCAGTTCCCCAACACGCTGCTGGGCAACCCTAAGAAACGCATGCGCTACTTC
GACCCCCTGAGGAACGAGTACTTCTTCGACCGCAACCGGCCCAGCTTCGACGCCATCCTCTACTACTACCA
GTCCGGCGGCCGCCTGCGGAGGCCGGTCAACGTGCCCCTGGACATGTTCTCCGAGGAGATCAAGTTTTAC
GAGTTGGGCGAGGAGGCCATGGAGAAGTTCCGGGAGGACGAGGGCTTCATCAAGGAGGAGGAGCGCCCT
CTGCCCGAGAAGGAGTACCAGCGCCAGGTGTGGCTGCTCTTCGAGTACCCCGAGAGCTCGGGGCCCGCCA
GGGTCATCGCCATCGTCTCCGTCATGGTCATCCTCATCTCCATCGTCATCTTTTGCCTGGAGACGCTCCCCG
AGCTGAAGGATGACAAGGACTTCACGGGCACCGTCCACCGCATCGACAACACCACGGTCATCTACAATTCC
AACATCTTCACAGACCCCTTCTTCATCGTGGAAACGCTGTGTATCATCTGGTTCTCCTTCGAGCTGGTGGTG
CGCTTCTTCGCCTGCCCCAGCAAGACGGACTTCTTCAAAAACATCATGAACTTCATAGACATTGTGGCCATC
ATTCCTTATTTCATCACGCTGGGCACCGAGATAGCTGAGCAGGAAGGAAACCAGAAGGGCGAGCAGGCCAC
CTCCCTGGCCATCCTCAGGGTCATCCGCTTGGTAAGGGTTTTTAGAATCTTCAAGCTCTCCCGCCACTCTAA
GGGCCTCCAGATCCTGGGCCAGACCCTCAAAGCTAGTATGAGAGAGCTAGGGCTGCTCATCTTTTTCCTCTT
CATCGGGGTCATCCTGTTTTCTAGTGCAGTGTACTTTGCCGAGGCGGAAGAAGCTGAGTCGCACTTCTCCA
GTATCCCCGATGCTTTCTGGTGGGCGGTGGTGTCCATGACCACTGTAGGATACGGTGACATGTACCCTGTG
ACAATTGGAGGCAAGATCGTGGGCTCCTTGTGTGCCATCGCTGGTGTGCTAACAATTGCCCTGCCCGTACC
TGTCATTGTGTCCAATTTCAACTATTTCTACCACCGAGAAACTGAGGGGGAAGAGCAGGCTCAGTTGCTCCA
CGTCAGTTCCCCTAACTTAGCCTCTGACAGTGACCTCAGTCGCCGCAGTTCCTCTACTATGAGCAAGTCTGA
GTACATGGAGATCGAAGAGGATATGAATAATAGCATAGCCCATTATAGACAGGTCAATATCAGAACTGCCAA
TTGCACCACTGCTAACCAAAACTGCGTTAATAAGAGCAAGCTACTGACCGATGTTTAAAAAACAAAGGCAAG
CAAACAAAAAAGCCCCACTTAGCAGCTCAAAAGACTTAAAAAACAAAACAGAAAACCTAGTGACTCATGTCAC
GCTTTGTAGATACTTTACTAAGTAGACTTGGAATGCTCTATTTAACTGTCAATGCGTTGTTGCATTGAGGATTT
TGGGGGTGGTGAACCAGAAGCTTTCAAGATCCATGACAAAATAAACTATTTTCCTTTTATTAAAAAATGGGAA
AAGAGAGAGTATTTTCTAAAACTGGCTTAAAAAGATTCAGTCCACGAACTAGTCTAGGTAAAATAATAATCATA
TGCTTCCCCAAACTGAAACATTTTTAATGCTTTGGTTTCTTTAACTTTTTTAAAAACTCAGAACAAGATGATCA
CTTAGAAATATGAAATTGAAATTCGCATGGGACTCCAGTAAAACATCTTTGCAAACTGCGTAGCACATTGAAG
ACAGTGCATCAGATGTATTATATGTAACATGATAGACCAGCCAAAATGGACAATGAATAGATATTTTTATTTCG
ATCAACTGAACTGCATATTACAAGGTGAAAAAAGAAAACTCCGATTACTTAAGACTGGTTCACAAAGCACCTT
ATAAATTGGATACTGGTCCTGATCTGTAGGGATTTCCCCCTGGGCCCATTCTCTTTCTAATCCAGATTATTCT
CTAAGAAAAAGTTAACTGAATTAAATTAATTGATTCTTCTGCAGTGCCGCTAAATGGTCTCAACTGCAGATGA
GCCAAATACAGGTCTTTTCTCACCAGGCCTGCACTCCGACCCCTGGCTTTCAGAACTGGATGTAAAACCTTA
GCCTCCTTATTGCAAGAGAGCACAAATGAAGTTAAATGTAAGCATGTTTGAATCTGATACAATTTATTTTATAA
TCGCATGCTGAGAAGTTAACCCAGACAATAGGGGATAAGCTTAAGTTGAAATCGATTCTTCTAAAAATAGATC
CTTTTTCATTTGCATTCACCAAAAGTGCACTCCTCCATTTATTAACTATTTTATTAGTAAATAAAGTACTGTATT
TAAGTGCATATGTTAGTCAGATGGGAACAATAACTTTTTGGAGCTCAAAGCATGTTCTCTTATTCAGCATTAT
GGCCTATTTGACTAAGATGTACCTTGAATTAATTAATGCATGATTTCAGTAATAAAAATTTTAAAAGTAATAAAA
ATTACAAGTCTGTGGGGTGAAAGGCCCAATAGAAATTATGGGGGGTGGGGGTGGGGGCACTCAGTCAATTT
TCCTGCCTTTGCTCAGGGAAATACCAGGTTTTTGTGCAGGTATAGGCGGAGAGAGGACCAATATGCCCATC
CCTTAAAGGGAAGCCATGTGAAAAACTCAATAAGTCATCAAAGTACATATAGCAACACCTAAGAACAAGTATT
CTTTCTAGCTGAAGACAAACACAAGCAACACAAACAAGCAAACAAACAAACAAACAAAAAGGTGCAATACTG
CATGTTTTTTGGTGCATTCTTAGGATGTAAATGAAAATGTTTCTCTATTATATGCATCCGAAGCAGAGCTGATT
TTTTTTTCTTTGCAGTCATTCTTTGAAGTCTGTAGAGACTTCAGCCCTCCCCTTGAGGCTCCCTGAAGAAACT
AAACCAATTGATTTAATAGTTGCTTAGTGCCTTTATCCTGTACCCACAGTGAACTGTAGAAAGTGCCTCCTTA
ACACAGCTGAGAAGTTAGGTAGCAAAAGTGGGGAAGGGTTGGGGCACAGACCTTTTGCTTTTTCTTTTTCCA
TTCTCGCTCTCTCATTTCACCACTGTGAGAAGACCACACCACCCTAAACCCTGGAGAGGAGAGACCCAGGA
GGGTGCTGTCTCTCTGGCCATCTACTAGCATTGGTCCCTTTGACAGCCTGACGCTGGATGTGAACTGAGAC
CCATCTTTGAACTGGACATGAACTGTGAACTTGTTTTTTCCTCTCTCCACCAAAGCCAAGATAAACTTTTTGG
GAATTTGTTTCCTATCGAGGGCCACTTTGGACACACAAGGCTTCCTCAGGTCCAGTGTAGTGCTCCTGGCAC
CTTTCCTTATTTTTTTCTCTGTCGGTAACAGCACTTTGCAAATCTCTCTGACGGTCCAGTCTTTTCAGGCATTG
TTGTGGATGTGGGAACACTCAGTTCATAATAACCTTTCCTAGGCCTTCCCTCCTGGTCTACCCCTTTCAGATA
TTTCCTGATGCCCCTATGATCTTCCCACCTGGCAGTCACTTCACAGGTTGAACATCTAACTTCTGCTGCCCC
CCCGTTACCCAGCCCAGAGAATGGTGGGGACCCTGTTCCTGGCTGAAAGAGAGCCACGGAACACAGGCCT
CTGGAGCTCGGCAGCTGCCCACCGGTGGAGAGGTACTCACAGCCTTTCAAAGGACCCTGAGGTGGGGAAT
CTTCATTCTGCACTTAGCATGTGGCTGCCTGTTACCTGACATTCTGGCCCAGCTTCTTCTGAAAATCTGTCCT
GCTTCTCCCACCCCTTTCCCCCATCCTGTCCTAGAACTAGCAGTGAGGCAATCACCCTAGAAACTCGAGTTA
CACCCATTCTGGCTAACTCGATTAAAAGAAAGAACATGGATATCTTATTTTCCTATGTGACTTTTGTGAATCTG
TGAGATGAACACAACACACATCGTGGAAGATGAGGGGCCAAGAACTGCGCAGCATCCGACTACAGGGCATT
AAACCCTCCCATGTGATGTCTCCTTCTCGTCTGAACCTTAACTCATTCTGGCGATTCCTTTCCTACTTGCTTA
AAAATCCCCAATTAGGAAAAAAATGCCAACCTCCTCCCAGGCTTGGGGTTATCTTGATCTTTCATTTCAACGT
TGAGGTCTAGTGCACACAGAACTTGAAACACCGCACTCTGTCAACAGCAATAATCCACTCAGTACTGTGGGA
TGGATGGGTTAATGGATGAGAAAACGGCACCAAGACTGACTTTGGGTATTTGGTATATTTACCGTGGTTACA
CTCTGGACTGGTTGATTCTTCTGCATGTGATATAGAAAAAAGTGCTGCATGCGGTGAACCTGTCAGTCTGGG
ACTGGGGGTGAGTTGTTGTTATGAGTTTGGGGTGGTGTGAGGAAAGTGAATGCTGCTTAGCTCATCTGTAG
CTCATTGCAATGAATTCAGTAAGAATGGAGTACAGGGATTATCTGTGTAGCATAGGCATGCAATGTTTGACCA
AGCTCTTACCCTCGCACTGTAATGTGTTGAAATGTCTTTGTAGACCTGAAGGTGCACTTAACAAAACTGCCTA
TTAAGGGATGACTATTTTTTGGTTTACTTATTCATATTTATTTTAGCAGCCTTTTTACCTTTTCTTCCCCTTCCT
AGGCATGGAGCTGTAACAGCTCATGTCCTGACTATGTGTTTTCTCCAGAAGGAGAAGACTTCTGATGTGCTG
ATAGCTATAATTCCTCTCTTCCAGTCTATTGGGGCCCTAGTTCAATAGGGTGGCAATAGAAGAGTTGGTCAC
ACCAGGGCTGTTAGCCATCCCAGAATCTCTGAAGTGGTTAACTCACCTGAAGTGATCTGAATCAGAGAGACC
AAAGACATTCATTTCCTCTGTCCTCAGATTTCTAGAAGACAAATTCTAGCCAGGAAAACTTTCTGTTTTTGCAT
CTCCCTTTTCCCATTTGTGCCCAACTCCCCTTGCACTCCCTGGAGACTTGAGTTCTGATTTTCAGTTATATCA
GCTCATTGGGAGTGTGTTCGTGGGTGAGCCTTGCAGAATCAGATAAACCAATAGCAAGTCCTTCTCCAGGAT
TCTGGCCAGTGGGCAGTCATTTCCCTGAAATGAATTGTAGGGCAGGTTTAGATTCCACTATGGTGGACGTGA
AGCACAGGTGGTGTCACAATCTTGATTTGCTTGAGAATTAAAAAAATACATGTAAATGGTAAATGAGGAATAC
ATTTTTTTAAAGTAGAAATTTGGTTTAGCCTATAAAAGGCCTTCTTCACATTGTGTATAGTTACACGTTTTTCAA
ATTAGGTACCATCTAAAAGTCAATAAAAATAGTTTCCTAGCCCTCTCATTTAATATAGGAGACCTCAAAACACA
TGCTTTTTAACAATTTTACATTTTAATTCTTAGTTTGGTAGATTGGATTGAAAGAAAGGAGAAAGAACATTAGC
AGAAGGCACTTTCCCATTTTCTTCCAGGAATGACTTATTCCTGGGGTGGTGAGAATTGGTGGGTGGTAACCA
TCCATAGTATAAAATTGTTAGAAAGAATATAAACTGCCAAACAAGCATGTTATCTTCAGGCTTTTCCAAGCAAG
AATGAAGTCTTTTGATGTTTATGTTCATTTTAAGAAGACAAACAAACTAAAATTTTAAGACCAGACACAACCCA
AGTTGAATTGTGATCTCAGGAGGTGAACTCTCATTCACCATGTGGCATATTACTACCTGTATTTATATCATGG
AATTTCAGGGTATATGTGAACATGTCTAGTATGACTCAGGTAAACCTTTAAAGAATGTATGTTACTTACCATTT
TTGTAAAGAAGCAAACAGGAGCTGAATTGTTAACCAAAACTGTTCCATTACCTTGGGTCACTGTGCAAACTAA
TTCAGGGTACAGATATAAAGTTTAGAAGCCTTGGGATTAAATGCCAGCCTCTTTGCTCAGGCAAAATGACCC
TGGGCTTTCTTGGGAAGTCCAGCATGTATGTAAGGGGTGAGGCCCTGCTGACCTCAGGGCTTTTCGCTTTA
AGGAGATTAACCCTAGTCACAGTGATTTTCATTTAGGAGCTAACTAGGAGTTTACTCTTTATGACGTGAAACT
TCAAAGAGTATAGAAAACTTTTGTACCACAATCAAAGAAGAAGAAATGGTGTATGGAAAGAAAACAAAACAAA
ACAAGAAATCTCTTGTAAAATATTCCAGGTCAAAGTTGTCTCCTCTCCAAACCTTGCAGAAGCACCTTTCTTC
TCTTCAGCGCACTGTTTTGGGACTGTTTATGCAGCAGATGTAAGTAGACAACATGGACTCCATGTGACATGC
CTCTAATAGTAAAGATAAAGTATTACTGAGGTTAAAAATAAAAATTGAGTAGTATTAATTTAAAGTGCACCATC
AGGACAACAAACCATTTAAGCTGAAAAAACGCTATTTTATTTCTTGAGTTTGCCAGTTGCTTCCACCTTGAGTT
AAGGACGTGTCTCATCTTCACCTACTGCGCATTCTCCCTTCTCTAACTGTGTAATATGTCAGGTCAAGGACAT
TGAATGTTATGAATTGAGAACCTAATTGATGCGCATAGTTTTCATCTATGCAATTTTACTTGCTTCTGTCACTT
TACGATCTGTTCATATTTGGCATCAATTAAAGATACTTTTTAAGGATCTTATCAAGGAA
Amino acid sequence of Kv1 gene product (SEQ ID NO: 6)
MTVMSGENVDEASAAPGHPQDGSYPRQADHDDHECCERVVINISGLRFETQLKTLAQFPNTLLGNPKKRMRYF
DPLRNEYFFDRNRPSFDAILYYYQSGGRLRRPVNVPLDMFSEEIKFYELGEEAMEKFREDEGFIKEEERPLPEKE
YQRQVWLLFEYPESSGPARVIAIVSVMVILISIVIFCLETLPELKDDKDFTGTVHRIDNTTVIYNSNIFTDPFFIVETLCI
IWFSFELVVRFFACPSKTDFFKNIMNFIDIVAIIPYFITLGTEIAEQEGNQKGEQATSLAILRVIRLVRVFRIFKLSRHSK
GLQILGQTLKASMRELGLLIFFLFIGVILFSSAVYFAEAEEAESHFSSIPDAFWWAVVSMTTVGYGDMYPVTIGGKI
VGSLCAIAGVLTIALPVPVIVSNFNYFYHRETEGEEQAQLLHVSSPNLASDSDLSRRSSSTMSKSEYMEIEEDMNN
SIAHYRQVNIRTANCTTANQNCVNKSKLLTDV
Nucleotide sequence of tetanus toxin light chain gene (SEQ ID NO: 7)
(GenBank: L19522.1)
GAATTCGCAATTAAGGAGATAATAGGTATGCCGATCACCATCAACAACTTCCGTTACTCCGACCCGGTTAAC
AACGACACCATCATCATGATGGAACCGCCGTACTGCAAAGGTCTAGACATCTACTACAAAGCCTTCAAAATC
ACCGACCGTATCTGGATCGTTCCGGAACGTTACGAATTTGGTACCAAACCGGAAGACTTCAACCCGCCGTC
CTCCCTGATCGAAGGTGCTTCCGAATACTACGACCCGAACTACCTGCGTACCGACTCCGACAAAGACCGTTT
CCTGCAGACCATGGTTAAACTGTTCAACCGTATCAAAAACAACGTTGCTGGTGAAGCTCTGCTGGACAAAAT
CATCAACGCTATCCCGTACCTGGGTAACTCCTACTCCCTGCTGGACAAATTCGACACCAACTCCAACTCCGT
TTCCTTCAACCTGCTGGAACAGGATCCGTCCGGTGCTACCACCAAATCCGCTATGCTGACCAACCTGATCAT
CTTCGGTCCGGGTCCGGTTCTGAACAAAAACGAAGTTCGTGGTATCGTTCTGCGTGTTGACAACAAAAACTA
CTTCCCGTGCCGTGACGGTTTCGGTTCCATCATGCAGATGGCTTTCTGCCCGGAATACGTACCGACCTTCG
ACAACGTTATCGAAAACATCACCTCCCTGACCATCGGTAAATCCAAATACTTCCAGGACCCGGCTCTGCTGC
TGATGCACGAACTGATCCACGTTCTGCACGGTCTGTACGGCATGCAGGTTTCCTCCCACGAAATCATCCCGT
CCAAACAGGAAATCTACATGCAGCACACCTACCCGATCTCCGCTGAAGAGCTCTTCACCTTCGGTGGTCAG
GACGCTAACCTGATCTCCATCGATATCAAAAACGACCTGTACGAAAAAACCCTGAACGACTACAAAGCTATC
GCTAACAAACTGTCCCAGGTTACCTCCTGCAACGACCCGAACATCGACATCGACTCCTACAAACAGATCTAT
CAGCAGAAGTATCAGTTTGATAAGGATTCCAACGGCCAGTATATAGTGAACGAGGATAAGTTTCAGATACTG
TATAACTCCATAATGTATGGCTTTACTGAGATAGAGCTGGGCAAGAAGTTTAACATAAAGACTCGCTTAAGCT
ATTTTTCCATGAACCACGATCCTGTGAAGATACCTAACCTGCTGGATGATACTATATATAACGATACTGAGGG
CTTTAACATAGAGTCCAAGGATCTGAAGTCCGAGTATAAGGGCCAGAACATGAGGGTGAACACTAACGCCTT
TAGGAACGTCGACGGTTCCGGTCTGGTTTCCAAACTGATCGGTCTGTGCAAAAAAATCATCCCGCCGACCAA
CATCCGTGAAAACCTGTACAACCGTACCGCTTAATTTAAAGCAGCTGCGATCGTAACTAAGTAATAAACGGT
CGTAACACAACAATTGCATTCCGGGGTTTTATGTTTCAGGTTGGGGGTGGTAAGCTT
Amino acid sequence of tetanus toxin light chain gene product (SEQ ID NO: 8)
MPITINNFRYSDPVNNDTIIMMEPPYCKGLDIYYKAFKITDRIWIVPERYEFGTKPEDFNPPSSLIEGASEYYDPNYL
RTDSDKDRFLQTMVKLFNRIKNNVAGEALLDKIINAIPYLGNSYSLLDKFDTNSNSVSFNLLEQDPSGATTKSAMLT
NLIIFGPGPVLNKNEVRGIVLRVDNKNYFPCRDGFGSIMQMAFCPEYVPTFDNVIENITSLTIGKSKYFQDPALLLM
HELIHVLHGLYGMQVSSHEIIPSKQEIYMQHTYPISAEELFTFGGQDANLISIDIKNDLYEKTLNDYKAIANKLSQVTS
CNDPNIDIDSYKQIYQQKYQFDKDSNGQYIVNEDKFQILYNSIMYGFTEIELGKKFNIKTRLSYFSMNHDPVKIPNLL
DDTIYNDTEGFNIESKDLKSEYKGQNMRVNTNAFRNVDGSGLVSKLIGLCKKIIPPTNIRENLYNRTA
Nucleotide sequence of hM4Di gene (SEQ ID NO: 9)
ATGGCCAACTTCACACCTGTCAATGGCAGCTCGGGCAATCAGTCCGTGCGCCTGGTCACGTCATCATCCCA
CAATCGCTATGAGACGGTGGAAATGGTCTTCATTGCCACAGTGACAGGCTCCCTGAGCCTGGTGACTGTCG
TGGGCAACATCCTGGTGATGCTGTCCATCAAGGTCAACAGGCAGCTGCAGACAGTCAACAACTACTTCCTCT
TCAGCCTGGCGTGTGCTGATCTCATCATAGGCGCCTTCTCCATGAACCTCTACACCGTGTACATCATCAAGG
GCTACTGGCCCCTGGGCGCCGTGGTCTGCGACCTGTGGCTGGCCCTGGACTGCGTGGTGAGCAACGCCTC
CGTCATGAACCTTCTCATCATCAGCTTTGACCGCTACTTCTGCGTCACCAAGCCTCTCACCTACCCTGCCCG
GCGCACCACCAAGATGGCAGGCCTCATGATTGCTGCTGCCTGGGTACTGTCCTTCGTGCTCTGGGCGCCTG
CCATCTTGTTCTGGCAGTTTGTGGTGGGTAAGCGGACGGTGCCCGACAACCAGTGCTTCATCCAGTTCCTG
TCCAACCCAGCAGTGACCTTTGGCACAGCCATTGCTGGCTTCTACCTGCCTGTGGTCATCATGACGGTGCT
GTACATCCACATCTCCCTGGCCAGTCGCAGCCGAGTCCACAAGCACCGGCCCGAGGGCCCGAAGGAGAAG
AAAGCCAAGACGCTGGCCTTCCTCAAGAGCCCACTAATGAAGCAGAGCGTCAAGAAGCCCCCGCCCGGGG
AGGCCGCCCGGGAGGAGCTGCGCAATGGCAAGCTGGAGGAGGCCCCCCCGCCAGCGCTGCCACCGCCAC
CGCGCCCCGTGGCTGATAAGGACACTTCCAATGAGTCCAGCTCAGGCAGTGCCACCCAGAACACCAAGGA
ACGCCCAGCCACAGAGCTGTCCACCACAGAGGCCACCACGCCCGCCATGCCCGCCCCTCCCCTGCAGCCG
CGGGCCCTCAACCCAGCCTCCAGATGGTCCAAGATCCAGATTGTGACGAAGCAGACAGGCAATGAGTGTGT
GACAGCCATTGAGATTGTGCCTGCCACGCCGGCTGGCATGCGCCCTGCGGCCAACGTGGCCCGCAAGTTC
GCCAGCATCGCTCGCAACCAGGTGCGCAAGAAGCGGCAGATGGCGGCCCGGGAGCGCAAAGTGACACGA
ACGATCTTTGCCATTCTGCTGGCCTTCATCCTCACCTGGACGCCCTACAACGTCATGGTCCTGGTGAACACC
TTCTGCCAGAGCTGCATCCCTGACACGGTGTGGTCCATTGGCTACTGGCTCTGCTACGTCAACAGCACCAT
CAACCCTGCCTGCTATGCTCTGTGCAACGCCACCTTTAAAAAGACCTTCCGGCACCTGCTGCTGTGCCAGTA
TCGGAACATCGGCACTGCCAGGCGGGATCCACCGGTC
Amino acid sequence of hM4Di gene product (SEQ ID NO: 10)
MANFTPVNGSSGNQSVRLVTSSSHNRYETVEMVFIATVTGSLSLVTVVGNILVMLSIKVNRQLQTVNNYFLFSLAC
ADLIIGAFSMNLYTVYIIKGYWPLGAVVCDLWLALDCVVSNASVMNLLIISFDRYFCVTKPLTYPARRTTKMAGLMIA
AAWVLSFVLWAPAILFWQFVVGKRTVPDNQCFIQFLSNPAVTFGTAIAGFYLPVVIMTVLYIHISLASRSRVHKHRP
EGPKEKKAKTLAFLKSPLMKQSVKKPPPGEAAREELRNGKLEEAPPPALPPPPRPVADKDTSNESSSGSATQNT
KERPATELSTTEATTPAMPAPPLQPRALNPASRWSKIQIVTKQTGNECVTAIEIVPATPAGMRPAANVARKFASIA
RNQVRKKRQMAARERKVTRTIFAILLAFILTWTPYNVMVLVNTFCQSCIPDTVWSIGYWLCYVNSTINPACYALCN
ATFKKTFRHLLLCQYRNIGTARRDPPV
Nucleotide sequence of Sequence A (SEQ ID NO: 11)
ATGGCTGCCGATGGTTATCTTCCAGATTGGCTCGAGGACAACCTCTCTGAGGGCATTCGCGACTGGTGGGA
CTTGAAACCTGGAGCCCCGAAACCCAAAGCCAACCAGCAAAAGCAGGACGACGGCCGGGGTCTGGTGCTT
CCTGGCTACAAGTACCTCGGACCCTTTAACGGACTCGACAAGGGGGAGCCCGTCAACGCGGCGGATGCAG
CGGCCCTCGAGCACGACAAGGCCTACGACCAGCAGCTCAAAGCGGGTGACAATCCGTACCTGCGGTATAA
CCACGCCGACGCCGAGTGTCAGGAGCGTCTGCAAGAAGATACGTCTTTTGGGGGCAACCTCGGGCGAGCA
GTCTTCCAGGCCAAGAAGAGGGTACTCGAACCTTTTGGTCTGGCTGAGGAAGGTGCTAAGACGGCTCCTGG
AAAGAAACGTCCGGTAGAGCAGTCGCCACAAGAGCCAGACTCCTCCTCGGGCATTGGCAAGACAGGCCAG
CAGCCCGCTAAAAAGAGACTCAATTTTGGTCAGACCGGCGACTCAGAGTCAGTCCCCGACCCACAACCTCT
CGGAGAACCTCCAGCAACCCCCGCTGCTGTGGGACCTACTACAATGGCTTCAGGCGGTGGCGCACCAATG
GCAGACAATAATGAAGGCGCCGACGGAGTGGGTAATGCCTCAGGAAATTGGCATTGCGATTCCACATGGCT
GGGCGACAGAGTCATCACCACCAGCACCCGAACATGGGCCTTGCCCACCTATAACAACCACCTCTACAAGG
AAATCTCCAGTGCTTCAACGGGGGCCAGCAACGACAACCACTACTTCGGCTACAGCACCCCCTGGGGGTAT
TTTGATTTCAACAGATTCCACTGCCATTTCTCACCACGTGACTGGCAGCGACTCATCAACAACAATTGGGGAT
TCCGGCCCAAGAGACTCAACTTCAAGCTCTTCAACATCCAAGTCAAGGAGGTCACGACGAATGATGGCGTC
ACGACCATCGCTAATAACCTTACCAGCACGGTTCAAGTCTTCTCGGACACGGAATACCAGTTGCCGTACGTC
CTCGGCTCTGCGCACCAGGGCTGCCTCCCTCCGTTCCCGGCGGACGTGTTCATGATTCCGCAGTACGGCT
ACCTAACGCTCAACAATGGCAGCCAGGCAGTGGGACGGTCATCCTTTTACTGCCTGGAATATTTCCCATCGC
AGATGATGAGAACGGGCAATAACTTTACCTTCAGCTACACATTCGAGGACGCGCCTTTCCACAGCAGCTACG
CGCACAGCCAGAGCCTGGACCGGCTGATGAATCCTCTCATCGACCAGTACTTGTATTACCTGAACAGAACTC
AGAATCAGTCCGGAAGTGCCCAAAACAAGGACTTGCTGTTTAGCCGGGGGTCTCCAGCTGGCATGTCTGTT
CAGCCCAAAAACTGGCTACCTGGACCCTGTTACCGGCAGCAGCGCGTTTCTAAAACAAAAACAGACAACAA
CAACAGCAACTTTACCTGGACTGGTGCTTCAAAATATAACCTTAATGGGCGTGAATCTATAATCAACCCTGGC
ACTGCTATGGCCTCACACAAAGACGACAAAGACAAGTTCTTTCCCATGAGCGGTGTCATGATTTTTGGAAAG
GAGAGCGCCGGAGCTTCAAACACTGCATTGGACAATGTCATGATCACAGACGAAGAGGAAATCAAAGCCAC
TAACCCCGTGGCCACCGAAAGATTTGGGACTGTGGCAGTCAATCTCCAGAGCAGCAGCACAGACCCTGCGA
CCGGAGATGTGCATGTTATGGGAGCCTTACCTGGAATGGTGTGGCAAGACAGAGACGTATACTTGCAGGGT
CCTATTTGGGCCAAAATTCCTCACACGGATGGACACTTTCACCCGTCTCCTCTCATGGGCGGCTTTGGACTT
AAGCACCCGCCTCCTCAGATCCTCATCAAAAACACGCCTGTTCCTGCGAATCCTCCGGCAGAGTTTTCGGCT
ACAAAGTTTGCTTCATTCATCACCCAGTATTCCACAGGACAAGTGAGCGTGGAGATTGAATGGGAGCTGCAG
AAAGAAAACAGCAAACGCTGGAATCCCGAAGTGCAGTATACATCTAACTATGCAAAATCTGCCAACGTTGAT
TTCACTGTGGACAACAATGGACTTTATACTGAGCCTCGCCCCATTGGCACCCGTTACCTCACCCGTCCCCTG
TAA
Amino acid sequence of Sequence A (SEQ ID NO: 12)
MAADGYLPDWLEDNLSEGIRDWWDLKPGAPKPKANQQKQDDGRGLVLPGYKYLGPFNGLDKGEPVNAADAAA
LEHDKAYDQQLKAGDNPYLRYNHADAECQERLQEDTSFGGNLGRAVFQAKKRVLEPFGLAEEGAKTAPGKKRP
VEQSPQEPDSSSGIGKTGQQPAKKRLNFGQTGDSESVPDPQPLGEPPATPAAVGPTTMASGGGAPMADNNEG
ADGVGNASGNWHCDSTWLGDRVITTSTRTWALPTYNNHLYKEISSASTGASNDNHYFGYSTPWGYFDFNRFHC
HFSPRDWQRLINNNWGFRPKRLNFKLFNIQVKEVTTNDGVTTIANNLTSTVQVFSDTEYQLPYVLGSAHQGCLPP
FPADVFMIPQYGYLTLNNGSQAVGRSSFYCLEYFPSQMMRTGNNFTFSYTFEDAPFHSSYAHSQSLDRLMNPLI
DQYLYYLNRTQNQSGSAQNKDLLFSRGSPAGMSVQPKNWLPGPCYRQQRVSKTKTDNNNSNFTWTGASKYNL
NGRESIINPGTAMASHKDDKDKFFPMSGVMIFGKESAGASNTALDNVMITDEEEIKATNPVATERFGTVAVNLQS
SSTDPATGDVHVMGALPGMVWQDRDVYLQGPIWAKIPHTDGHFHPSPLMGGFGLKHPPPQILIKNTPVPANPPA
EFSATKFASFITQYSTGQVSVEIEWELQKENSKRWNPEVQYTSNYAKSANVDFTVDNNGLYTEPRPIGTRYLTRPL
AAV6 Capsid DNA sequence (SEQ ID NO: 13)
ATGGCTGCCGATGGTTATCTTCCAGATTGGCTCGAGGACAACCTCTCTGAGGGCATTCGCGAGTGGTGGGA
CTTGAAACCTGGAGCCCCGAAACCCAAAGCCAACCAGCAAAAGCAGGACGACGGCCGGGGTCTGGTGCTT
CCTGGCTACAAGTACCTCGGACCCTTCAACGGACTCGACAAGGGGGAGCCCGTCAACGCGGCGGATGCAG
CGGCCCTCGAGCACGACAAGGCCTACGACCAGCAGCTCAAAGCGGGTGACAATCCGTACCTGCGGTATAA
CCACGCCGACGCCGAGTTTCAGGAGCGTCTGCAAGAAGATACGTCTTTTGGGGGCAACCTCGGGCGAGCA
GTCTTCCAGGCCAAGAAGAGGGTTCTCGAACCTTTTGGTCTGGTTGAGGAAGGTGCTAAGACGGCTCCTGG
AAAGAAACGTCCGGTAGAGCAGTCGCCACAAGAGCCAGACTCCTCCTCGGGCATTGGCAAGACAGGCCAG
CAGCCCGCTAAAAAGAGACTCAATTTTGGTCAGACTGGCGACTCAGAGTCAGTCCCCGACCCACAACCTCT
CGGAGAACCTCCAGCAACCCCCGCTGCTGTGGGACCTACTACAATGGCTTCAGGCGGTGGCGCACCAATG
GCAGACAATAACGAAGGCGCCGACGGAGTGGGTAATGCCTCAGGAAATTGGCATTGCGATTCCACATGGCT
GGGCGACAGAGTCATCACCACCAGCACCCGAACATGGGCCTTGCCCACCTATAACAACCACCTCTACAAGC
AAATCTCCAGTGCTTCAACGGGGGCCAGCAACGACAACCACTACTTCGGCTACAGCACCCCCTGGGGGTAT
TTTGATTTCAACAGATTCCACTGCCATTTCTCACCACGTGACTGGCAGCGACTCATCAACAACAATTGGGGAT
TCCGGCCCAAGAGACTCAACTTCAAGCTCTTCAACATCCAAGTCAAGGAGGTCACGACGAATGATGGCGTC
ACGACCATCGCTAATAACCTTACCAGCACGGTTCAAGTCTTCTCGGACTCGGAGTACCAGTTGCCGTACGTC
CTCGGCTCTGCGCACCAGGGCTGCCTCCCTCCGTTCCCGGCGGACGTGTTCATGATTCCGCAGTACGGCT
ACCTAACGCTCAACAATGGCAGCCAGGCAGTGGGACGGTCATCCTTTTACTGCCTGGAATATTTCCCATCGC
AGATGCTGAGAACGGGCAATAACTTTACCTTCAGCTACACCTTCGAGGACGTGCCTTTCCACAGCAGCTACG
CGCACAGCCAGAGCCTGGACCGGCTGATGAATCCTCTCATCGACCAGTACCTGTATTACCTGAACAGAACT
CAGAATCAGTCCGGAAGTGCCCAAAACAAGGACTTGCTGTTTAGCCGGGGGTCTCCAGCTGGCATGTCTGT
TCAGCCCAAAAACTGGCTACCTGGACCCTGTTACCGGCAGCAGCGCGTTTCTAAAACAAAAACAGACAACAA
CAACAGCAACTTTACCTGGACTGGTGCTTCAAAATATAACCTTAATGGGCGTGAATCTATAATCAACCCTGGC
ACTGCTATGGCCTCACACAAAGACGACAAAGACAAGTTCTTTCCCATGAGCGGTGTCATGATTTTTGGAAAG
GAGAGCGCCGGAGCTTCAAACACTGCATTGGACAATGTCATGATCACAGACGAAGAGGAAATCAAAGCCAC
TAACCCCGTGGCCACCGAAAGATTTGGGACTGTGGCAGTCAATCTCCAGAGCAGCAGCACAGACCCTGCGA
CCGGAGATGTGCATGTTATGGGAGCCTTACCTGGAATGGTGTGGCAAGACAGAGACGTATACCTGCAGGGT
CCTATTTGGGCCAAAATTCCTCACACGGATGGACACTTTCACCCGTCTCCTCTCATGGGCGGCTTTGGACTT
AAGCACCCGCCTCCTCAGATCCTCATCAAAAACACGCCTGTTCCTGCGAATCCTCCGGCAGAGTTTTCGGCT
ACAAAGTTTGCTTCATTCATCACCCAGTATTCCACAGGACAAGTGAGCGTGGAGATTGAATGGGAGCTGCAG
AAAGAAAACAGCAAACGCTGGAATCCCGAAGTGCAGTATACATCTAACTATGCAAAATCTGCCAACGTTGAT
TTCACTGTGGACAACAATGGACTTTATACTGAGCCTCGCCCCATTGGCACCCGTTACCTCACCCGTCCCCTG
TAA
AAV6 Amino Acid Sequence (SEQ ID NO: 14)
MAADGYLPDWLEDNLSEGIREWWDLKPGAPKPKANQQKQDDGRGLVLPGYKYLGPFNGLDKGEPVNAADAAA
LEHDKAYDQQLKAGDNPYLRYNHADAEFQERLQEDTSFGGNLGRAVFQAKKRVLEPFGLVEEGAKTAPGKKRP
VEQSPQEPDSSSGIGKTGQQPAKKRLNFGQTGDSESVPDPQPLGEPPATPAAVGPTTMASGGGAPMADNNEG
ADGVGNASGNWHCDSTWLGDRVITTSTRTWALPTYNNHLYKQISSASTGASNDNHYFGYSTPWGYFDFNRFHC
HFSPRDWQRLINNNWGFRPKRLNFKLFNIQVKEVTTNDGVTTIANNLTSTVQVFSDSEYQLPYVLGSAHQGCLPP
FPADVFMIPQYGYLTLNNGSQAVGRSSFYCLEYFPSQMLRTGNNFTFSYTFEDVPFHSSYAHSQSLDRLMNPLID
QYLYYLNRTQNQSGSAQNKDLLFSRGSPAGMSVQPKNWLPGPCYRQQRVSKTKTDNNNSNFTWTGASKYNLN
GRESIINPGTAMASHKDDKDKFFPMSGVMIFGKESAGASNTALDNVMITDEEEIKATNPVATERFGTVAVNLQSSS
TDPATGDVHVMGALPGMVWQDRDVYLQGPIWAKIPHTDGHFHPSPLMGGFGLKHPPPQILIKNTPVPANPPAEF
SATKFASFITQYSTGQVSVEIEWELQKENSKRWNPEVQYTSNYAKSANVDFTVDNNGLYTEPRPIGTRYLTRPL
Amino Acid Sequence of capsid encoded by SEQ ID NO: 1 (SEQ ID NO: 15)
MAADGYLPDWLEDNLSEGIREWWDLKPGAPKPKANEQKQDDGRGLVLPGYKYLGPFNGLDKGEPVNAADAAA
LEHDKAYDQQLKAGDNPYLRYNHADAEFQERLQEDTSFGGNLGRAVFQAKKRVLEPLGLVEEGAKTAPGKKRP
VEQSPQEPDSSSGIGKTGQQPAKKRLNFGQTGDSESVPDPQPLGEPPATPAAVGPTTMASGGGAPMADNNEG
ADGVGNASGNWHCDSTWLGDRVITTSTRTWALPTYNNHLYKQISSASTGASNDNHYFGYSTPWGYFDFNRFHC
HFSPRDWQRLINNNWGFRPKRLNFKLFNIQVKEVTTNDGVTTIANNLTSTVQVFSDSEYQLPYVLGSAHQGCLPP
SPADVFMIPQYGYLTLNNGSQAVGRSSFYCLEYFPSQMLRTGNNFTFSYTFEDVPFHSSYAHSQSLDRLMNPLID
QYLYYLNRTQNQSGSAQYKDLLFSRGSPAGMSVQPKNWLPGPCYRQQRVSKTKTDNNNSNFTWTGASKYNLN
GRESIINPGTAMASHKDDKDKFFPMSGVMIFGKESAGASNTALDNVMITDEEEIKATNPVATERFGTVAVNLQSSS
TDPATGDVHVMGALPGMVWQDRDVYLQGPIWAKIPHTDGHFHPSPLMGGFGLKHPPPQILIKNTPVPANPPAEF
SATKFASFITQYSTGQVSVEIEWELQKENSKRWNPEVQYTSIYAKSANVDFTVDNNGLYTEPRPIGTRYLTRPL
Amino Acid Sequence of capsid encoded by SEQ ID NO: 2 (SEQ ID NO: 16)
MAADGYLPDWLEDNLSEGIREWWDLKPGAPKRKANQQKQDDGRGLVLPGYKYLGPFNGLDKGEPVNAADAAA
LEHDKAYDQQLKAGDNPYLRYNHADAEFQERLQEDTSFGGNLGRAVFQAKKRVLEPLGLVEEGAKTAPGKKRP
VEQSPQEPDSSSGIGKTGQQPAKKRLNFGQTGDSESVPDPQPLGEPPATPAAVGPTTMASGGGAPMADNNEG
ADGVGNASGNWHCDSTWMGDRVITTSTRTWALPTYNNHLYKQISSASTGASNDNHYFGYSTPWGYFDFNRFH
CHFSPRDWQRLINNNWGFRPKRLNFKLFNIQVKEVTTNDGVTTIANNLTSTVQVFSDSEYQLPYVLGSAHQGCLP
PFPADVFMIPQYGYQTLNNGSQAVGRSSFYCLEYFPSQMLRTGNNFTISYTFEDVPFHSSYAHSQSLDRLMNPLI
DQYLYYLNRTQNQSGSAQNKDLLFSRGSPAGMSVQPKNWLPGPCYRQQRVSKT*TDNNNSNFTWTGASKYNL
NGRESIINPGTAMASHKDDKDKFFPMSGVMIFGKESAGASNTALDNVMITDEEEIKATNPVATERFGTVAVNLQS
SSTDPATGDVHVMGALPGMAWQDRDVYLQGPIWAKIPHTDGHFHPSPLMGGFGLKHPPPQILIKNTPVPANPPA
EFSATKFASFITQYSTGQVSVEIEWELQKENSKRWNPEVQYTSNYAKSANVDFTVDNNGLYTEPRPIGTRYLTRPL

1. A method of screening for capsid-encoding nucleotide sequences of adeno-associated virus (“AAV”) particles capable of infecting neurons in a subject, the method comprising:
(i) providing a population of neurons, wherein said neurons are derived from induced pluripotent stem cells (“iPSCs”) or embryonic stem cells (“ESCs”);
(ii) contacting the population with a first plurality of test AAV particles;
(iii) isolating a first plurality of AAV particles that have infected the neurons; and
(iv) determining the capsid-encoding nucleotide sequences of the first plurality of AAV particles that have infected the neurons.

2. A method of screening for capsid-encoding nucleotide sequences of adeno-associated virus (“AAV”) particles capable of infecting a specific sub-compartment of neurons in a subject, the method comprising:
(i) providing a population of neurons, wherein said neurons are derived from induced pluripotent stem cells (“iPSCs”) or embryonic stem cells (“ESCs”);
(ii) contacting the population with a first plurality of test AAV particles;
(iii) isolating a first plurality of AAV particles that have infected the specific sub-compartment of the neurons; and
(iv) determining the capsid-encoding nucleotide sequences of the first plurality of AAV particles that have infected the neurons.

3. A method of screening for capsid-encoding nucleotide sequences of adeno-associated virus (“AAV”) particles capable of infecting neurons in a subject, the method comprising:
(i) providing a population comprising neurons,
wherein said neurons are derived from induced pluripotent stem cells (“iPSCs”) or embryonic stem cells (“ESCs”), and
wherein said neurons each have a first specific sub-compartment and a second specific sub-compartment;
(ii) disposing said neurons so that the first specific sub-compartments and second specific sub-compartments are distally separated from each other;
(iii) contacting the first specific sub-compartments with a first plurality of test AAV particles;
(iv) isolating a first plurality of AAV particles that have infected the second specific sub-compartments; and
(v) determining the capsid-encoding nucleotide sequences of the first plurality of AAV particles that have infected the second specific sub-compartment of neurons.

4. The method of claim 3, wherein:
(a) in step (ii), the first specific sub-compartments and second specific sub-compartments are grouped in different physical regions of one or more containers; and/or
(b) the first specific sub-compartments and second specific sub-compartments are grouped in different physical regions of one or more containers in a microfluidic chamber; and/or
(c) the first specific sub-compartments and second specific sub-compartments are connected by axons; and/or
(d) the one or more containers further comprise skeletal muscle cells, and/or myocytes, and/or sensory neurons.

5. The method of any of the above claims, wherein the neuron is a motor neuron.

6. The method of any of the above claims, further comprising, after the determination step:
(i) generating a second plurality of test AAV particles using the capsid-encoding nucleotide sequences of the first plurality of AAV particles that have infected the neurons;
(ii) repeating steps (i) to (iii) with the second plurality of test AAV capsids, in order to isolate a second plurality of AAV particles that have infected the neurons; and
(iii) determining the capsid-encoding nucleotide sequences of the second plurality of AAV particles that have infected the neurons,
wherein the capsid-encoding nucleotide sequences of the second plurality of AAV particles are more effective at infecting the neurons than the capsid-encoding nucleotide sequences of the first plurality of AAV particles.

7. The method of claim 6, wherein the second plurality of test AAV particles are generated by one or more of:
(i) random mutagenesis of the capsid-encoding nucleotide sequences of the first plurality of AAV particles that have infected the neurons;
ii) shuffling of the capsid-encoding nucleotide sequences of the first plurality of AAV particles that have infected the neurons; and
iii) insertion of targeted or random peptide sequences up to 25 amino acids in length at various regions in VP1, VP2 or VP3 of the capsid-encoding nucleotide sequences of the first plurality of AAV particles that have infected the neurons.

8. The method of any of the above claims, wherein the iPSCs or ESCs are derived from i) the subject, optionally a human subject; and/or ii) a skin sample of the subject; and/or iii) fibroblasts of the subject.

9. The method of any of the above claims, wherein step (i) comprises a step of deriving neurons from iPSCs or ESCs.

10. The method of any of the above claims, wherein the method is a method a screening for capsid—encoding nucleotide sequences of adeno-associated virus (“AAV”) particles capable of infecting neurons in a subject via intramuscular injection.

11. An adeno-associated virus (“AAV”) capsid-encoding nucleotide identified by a screening method of any of the above claims.

12. An adeno-associated virus (“AAV”) capsid-encoding nucleotide sequence having at least 50, 55, 60, 65, 70, 75, 80, 85, 90, 91, 92, 93, 94, 95, 96, 97, 98, 99, or 100% sequence identity to SEQ ID NO: 11.

13. An adeno-associated virus (“AAV”) capsid-encoding nucleotide sequence having at least 50, 55, 60, 65, 70, 75, 80, 85, 90, 91, 92, 93, 94, 95, 96, 97, 98, 99, or 100% sequence identity to SEQ ID NO: 1 or SEQ ID NO: 2.

14. A recombinant adeno-associated virus (“AAV”) expression vector comprising a capsid-encoding nucleotide sequence of any of claims 11-13.

15. The expression vector of claim 14, wherein the expression vector further comprises a transgene encoding a transgene product, wherein the transgene product is capable of altering the excitability of neurons when the expression vector is administered to a subject, optionally wherein the transgene product is capable of reducing the hyperexcitability of neurons when the expression vector is administered to a subject.

16. The expression vector of claim 15, wherein the transgene product is:
(a) capable of dampening neuron excitability when the expression vector is administered to a subject;
(b) capable of blocking synaptic transmission of neurons when the expression vector is administered to a subject;
(c) a receptor activated solely by a synthetic ligand (RASSL); or
(d) a designer receptor exclusively activated by designer drugs (DREADD).

17. The expression vector of any of claims 14-16, wherein the transgene or transgene product is:
(a) a KCC2 transgene or transgene product, optionally wherein:
(i) the KCC2 transgene has at least 50, 55, 60, 65, 70, 75, 80, 85, 90, 91, 92, 93, 94, 95, 96, 97, 98, 99, or 100% sequence identity to SEQ ID NO: 3; or
(ii) the KCC2 transgene product has at least 50, 55, 60, 65, 70, 75, 80, 85, 90, 91, 92, 93, 94, 95, 96, 97, 98, 99, or 100% sequence identity to SEQ ID NO: 4; or
(b) a Kv1 transgene or transgene product, optionally wherein:
(i) the Kv1 transgene has at least 50, 55, 60, 65, 70, 75, 80, 85, 90, 91, 92, 93, 94, 95, 96, 97, 98, 99, or 100% sequence identity to SEQ ID NO: 5; or
(ii) the Kv1 transgene product has at least 50, 55, 60, 65, 70, 75, 80, 85, 90, 91, 92, 93, 94, 95, 96, 97, 98, 99, or 100% sequence identity to SEQ ID NO: 6; or
(c) a tetanus toxin light chain transgene or transgene product, optionally wherein:
(i) the tetanus toxin light chain transgene has at least 50, 55, 60, 65, 70, 75, 80, 85, 90, 91, 92, 93, 94, 95, 96, 97, 98, 99, or 100% sequence identity to SEQ ID NO: 7; or
(ii) the tetanus toxin light chain transgene product has at least 50, 55, 60, 65, 70, 75, 80, 85, 90, 91, 92, 93, 94, 95, 96, 97, 98, 99, or 100% sequence identity to SEQ ID NO: 8; or
(d) an hM4Di transgene or transgene product, optionally wherein:
(i) the hM4Di transgene has at least 50, 55, 60, 65, 70, 75, 80, 85, 90, 91, 92, 93, 94, 95, 96, 97, 98, 99, or 100% sequence identity to SEQ ID NO: 9; or
(ii) the hM4Di transgene product has at least 50, 55, 60, 65, 70, 75, 80, 85, 90, 91, 92, 93, 94, 95, 96, 97, 98, 99, or 100% sequence identity to SEQ ID NO: 10.

18. The expression vector of any of claims 14-17, wherein the expression vector is an AAV2 expression vector or an AAV6 expression vector.

19. The expression vector of any of claims 14-18, wherein the expression vector further comprises:
(a) a neuron-specific promoter gene operably linked to the transgene; and/or
(b) a rep gene, optionally wherein the rep gene is an AAV2 rep gene; and/or
(c) a cap gene, optionally wherein the cap gene is an AAV2 cap gene; and/or
(d) an inverted terminal repeat, optionally wherein the inverted terminal repeat is an AAV2 inverted terminal repeat; and/or
(e) a gene encoding a viral packaging and/or envelope protein.

20. The expression vector of any of claims 14-19, wherein the expression vector is capable of altering the activity of targeted neurons in a subject, optionally via intramuscular injection.

21. An in vitro method of making viral particles comprising:
(i) transducing mammalian cells with an AAV expression vector of any of claims 14-20 and expressing viral packaging and envelope proteins necessary for particle formation in the cells; and
(ii) culturing the transduced cells in a culture medium, such that the cells produce viral particles that are released into the medium.

22. A viral particle comprising an AAV expression vector of any of claims 14-20.

23. A method of ameliorating or treating a neuromuscular or neuromotor disorder in a subject, comprising administering to the subject a therapeutically active amount of the AAV expression vector of any of claims 14-20, or a viral particle according to claim 22.

24. The method of claim 23, wherein the disorder is spasticity, amyotrophic lateral sclerosis, spinal muscular atrophy or other movement disorders such as dystonia.

25. The method of any of claims 23-24, wherein the AAV expression vector or viral particle is delivered intramuscularly, intravenously, intracranially, or intraspinally.

26. A method of ameliorating or treating a neuromuscular or neuromotor disorder in a subject, comprising administering to the subject a therapeutically active amount of an AAV expression vector of any of claims 14-20 or viral particle of claim 22, wherein the AAV expression vector or viral particle comprises the capsid-encoding nucleotide sequence of an AAV capsid identified by a screening method of any of claims 1-10, and wherein the iPSCs or ESCs used in the screening method have been derived from the subject.

27. The method of any of claims 23-26, wherein the iPSCs or ESCs used in the method have been derived from a skin sample of the subject.

28. The method of any of claims 23-27, wherein the AAV expression vector or viral particle is delivered intramuscularly, in order to infect neurons of a subject neuron retrogradely and alter the activity of the neurons in a subject.

29. An AAV expression vector of any of claims 14-20 or the viral particle of claim 22, for use in the method of any one of claims 23-28.

30. A kit comprising an AAV expression vector of any of claims 14-20 and one or more viral packaging and envelope expression vectors that encode viral packaging and envelope proteins necessary for particle formation when expressed in a cell.

31. A cell comprising an AAV expression vector of any of claims 14-20, optionally wherein the cell is a mammalian cell, further optionally wherein the mammalian cell is a HEK293 cell.

32. A capsid comprising an amino acid sequence having at least 50, 55, 60, 65, 70, 75, 80, 85, 90, 91, 92, 93, 94, 95, 96, 97, 98, 99, or 100% sequence identity to SEQ ID NO: 12, 15 or 16.
